(19) United States
(12) Patent Application Publication
(10) Pub. No.: US 2024/0209451 A1
(43) Pub. Date: Jun. 27, 2024

(54) CANCER DETECTION METHOD, KIT, AND SYSTEM

(71) Applicant: MIRONCOL DIAGNOSTICS, INC., Dover, DE (US)

(72) Inventors: Andrew ZHANG, San Diego, CA (US); Hai HU, Johnstown, PA (US)

(73) Assignee: MIRONCOL DIAGNOSTICS, INC., Dover, DE (US)

(21) Appl. No.: 18/536,108

(22) Filed: Dec. 11, 2023

Related U.S. Application Data
(63) Continuation-in-part of application No. PCT/US2022/032423, filed on Jun. 7, 2022.
(60) Provisional application No. 63/208,506, filed on Jun. 9, 2021.
(60) Provisional application No. 63/538,292, filed on Sep. 14, 2023.

(51) Int. Cl. C12Q 1/6886; C12N 15/10; C12Q 1/6874; G16H 20/10; G16H 50/20

(52) U.S. Cl. CPC C12Q 1/6886; C12N 15/1096; C12Q 1/6874; G16H 20/10; G16H 50/20; C12Q 2600/158

(57) ABSTRACT

Provided herein include a method, kit and system capable of detecting one or multiple human cancers with high accuracy. After an expression profile of an miRNA biomarker set comprising one or more miRNAs is determined based on a liquid biopsy sample from a subject, a diagnostic index is calculated, based on which the subject can be classified as having cancer or not. In the miRNA biomarker set, each miRNA is picked from a 500-miRNA set. A 4-miRNA biomarker model demonstrates exceptionally high sensitivities of 99.0-100% for lung and gastric cancers, 83.0-99.0% for biliary tract, bladder, colorectal, esophageal, glioma, liver, pancreatic, and prostate cancers, and 68.2-72.0% for ovarian cancer and sarcoma, while maintaining 99.3% specificity.

### CROSS-REFERENCE TO RELATED APPLICATIONS

The present application is a continuation-in-part application of PCT/US2022/032423, filed Jun. 7, 2022, which claims the benefit of U.S. Provisional Application No. 63/208,506 filed on Jun. 9, 2021. The present application further claims the benefit of U.S. Provisional Application No. 63/538,292 filed on Sep. 14, 2023. The disclosures of these above applications are hereby incorporated by reference in their entireties.

### REFERENCE TO SEQUENCE LISTING SUBMITTED ELECTRONICALLY

The content of the electronically submitted sequence listing, file name top500miRNAs-new.xml, size 430,334 bytes, and date of creation Mar. 11, 2024, filed herewith, is incorporated herein by reference in its entirety.

### FIELD OF THE INVENTION

The present invention relates generally to the technical field of disease screening, detection and diagnosis, and more specifically relates to a method, a kit, a system, and a non-transitory storage medium for the detection of one or multiple human cancers.

### BACKGROUND

Despite the recent rapid development of diagnosis and treatment technologies, cancers have remained largely as a challenging and potentially lethal disease for humans. It is well appreciated that detection of cancer at early stages is critical to decrease cancer-related mortality as treatment is more likely to be successful at early stages. There is an urgent unmet need to develop a test capable of detecting multiple cancer types early and simultaneously, ideally noninvasive, such as a blood test, which has become the cornerstone of the so-called multi-cancer early detection (MCED) paradigm. Such a MCED test often requires very high specificity, preferably >99%, to ensure minimum false positives in order to be able to screen the at-risk general population.

Molecules such as microRNAs (i.e. miRNAs) may serve as biomarkers for MCED. miRNAs are small single-stranded non-coding RNA molecules of an average of 22 nucleotides long encoded by their corresponding genes in the human genome. The miRNAs function in negative post-transcriptional regulation of gene expression primarily by binding with complementary sequences in the 3′ untranslated region (3′ UTR) of mRNA molecules. miRNAs appear to regulate more than 50% human genes, and abnormal expression of miRNAs has been implicated in many human cancers. miRNAs are also abundant as extracellular circulating molecules released into circulation by tumor cells either through cell death or by exosome-mediated signaling. Combined with its remarkable stability in the blood and other body fluids, circulating cell-free miRNAs have the potential to serve as noninvasive biomarkers for cancer screening and diagnosis.

### SUMMARY

The present disclosure provides a multi-cancer detection approach (i.e. method, kit, and system) by means of an miRNA biomarker set consisting of at least one miRNA biomarker. The approach is substantially based on the expression profile of the miRNA biomarker set, which can be determined from a biological sample obtained from a human subject. Such a biological sample can notably be a liquid biopsy sample including a blood sample, a serum sample, a plasma sample, a urine sample, a saliva sample, or a spatum sample to thereby allow a non-invasive or minimum-invasive detection for the cancer. The approach can be employed to accurately and reliably detect whether a human subject has one of the cancers including lung cancer, biliary tract cancer, bladder cancer, colorectal cancer, esophageal cancer, gastric cancer, glioma cancer, liver cancer, pancreatic cancer, prostate cancer, ovarian cancer, and sarcoma.

In a first aspect, a method for detecting a cancer from a biological sample obtained from a subject is provided. The method substantially includes the following three steps (1)-(3):
Step (1): determining, from the biological sample, an expression profile of an miRNA biomarker set, wherein the miRNA biomarker set consists of at least one miRNA, and each of the at least one miRNA is from a 500-miRNA set as listed in Table 1.
Step (2): calculating a diagnostic index of the biological sample based on the expression profile of the miRNA biomarker set. The diagnostic index is calculated based on:

$$diagnostic index = {\sum }_{i = 1}^{n} {t}_{i} * {miRNA}_{i} ; \qquad ( I )$$

where n is the total number of the at least one miRNA in the miRNA biomarker set and is an integer greater than zero and smaller than or equal to 500, miRNAᵢ is the expression level of i^{th} miRNA in the miRNA biomarker set, i is an integer greater than zero and smaller than or equal to n; and tᵢ is a weight for the i^{th} miRNA.
Step (3): classifying the subject as having the cancer or not based on the value of the calculated diagnostic index. If the calculated diagnostic index is greater than or equal to a pre-determined threshold, the subject is classified as having the cancer; or if otherwise the subject is classified as not having the cancer.

Herein, the method is capable of achieving diagnostic accuracy having an AUC value greater than approximately 0.75 for detecting any one of the following twelve cancers including lung cancer, biliary tract cancer, bladder cancer, colorectal cancer, esophageal cancer, gastric cancer, glioma, liver cancer, ovarian cancer, pancreatic cancer, prostate cancer, and sarcoma.

As used herein, the miRNA biomarker set substantially contains at least one miRNAs, each listed in Table 1 below, which lists the top 500 miRNAs that have been identified to be able to achieve high accuracy in detecting multiple cancer types. The total number n of the at least one miRNA in the miRNA biomarker set can thus be 1, 2, 3, . . . , or 500. The expression profile of the miRNA biomarker set is substantially a dataset containing expression level data that has been determined for each and every miRNA member contained in the miRNA biomarker set.

The term “pre-determined threshold” is referred to as a cut-point value of the diagnostic index that can be used to determine with a given specificity/sensitivity if a subject has the cancer type or not. It is typically pre-determined based on an existing dataset comprising a range of diagnostic index values that have been obtained and calculated for an existing population of subjects known to have, and/or known to be absent of, the disease.

According to some embodiments, n is one, i.e., the miRNA biomarker set consists of any single one miRNA from the 500-miRNA set in Table 1 (i.e. any single one miRNA is picked from the 500-miRNA list), such that the method is capable of achieving diagnostic accuracy for detecting lung cancer having an AUC value greater than approximately 0.88, for detecting biliary tract cancer having an AUC value greater than approximately 0.88, for detecting bladder cancer having an AUC value greater than approximately 0.85, for detecting colorectal cancer having an AUC value greater than approximately 0.87, for detecting esophageal cancer having an AUC value greater than approximately 0.82, for detecting gastric cancer having an AUC value greater than approximately 0.89, for detecting glioma having an AUC value greater than approximately 0.81, for detecting liver cancer having an AUC value greater than approximately 0.83, for detecting ovarian cancer having an AUC value greater than approximately 0.78, for detecting pancreatic cancer having an AUC value greater than approximately 0.80, for detecting prostate cancer having an AUC value greater than approximately 0.87, or for detecting sarcoma having an AUC value greater than approximately 0.80.

According to some other embodiments, n is two, i.e., the miRNA biomarker set consists of any two miRNAs from the 500-miRNA set in Table 1 (i.e. any two miRNAs are picked from the 500-miRNA list), such that the method is capable of achieving diagnostic accuracy for detecting lung cancer having an AUC value greater than approximately 0.92, for detecting biliary tract cancer having an AUC value greater than approximately 0.91, for detecting bladder cancer having an AUC value greater than approximately 0.91, for detecting colorectal cancer having an AUC value greater than approximately 0.90, for detecting esophageal cancer having an AUC value greater than approximately 0.84, for detecting gastric cancer having an AUC value greater than approximately 0.92, for detecting glioma having an AUC value greater than approximately 0.84, for detecting liver cancer having an AUC value greater than approximately 0.85, for detecting ovarian cancer having an AUC value greater than approximately 0.82, for detecting pancreatic cancer having an AUC value greater than approximately 0.85, for detecting prostate cancer having an AUC value greater than approximately 0.90, or for detecting sarcoma having an AUC value greater than approximately 0.85.

According to some embodiments, n is three, i.e., the miRNA biomarker set consists of any three miRNAs from the 500-miRNA set in Table 1 (i.e. any three miRNAs are picked from the 500-miRNA list), such that the method is capable of achieving diagnostic accuracy for detecting lung cancer having an AUC value greater than approximately 0.95, for detecting biliary tract cancer having an AUC value greater than approximately 0.95, for detecting bladder cancer having an AUC value greater than approximately 0.93, for detecting colorectal cancer having an AUC value greater than approximately 0.93, for detecting esophageal cancer having an AUC value greater than approximately 0.86, for detecting gastric cancer having an AUC value greater than approximately 0.95, for detecting glioma having an AUC value greater than approximately 0.87, for detecting liver cancer having an AUC value greater than approximately 0.87, for detecting ovarian cancer having an AUC value greater than approximately 0.84, for detecting pancreatic cancer having an AUC value greater than approximately 0.86, for detecting prostate cancer having an AUC value greater than approximately 0.92, or for detecting sarcoma having an AUC value greater than approximately 0.86.

According to some embodiments, the method is capable of achieving diagnostic accuracy having an AUC value greater than approximately 0.80 for detecting lung cancer, biliary tract cancer, bladder cancer, colorectal cancer, esophageal cancer, gastric cancer, glioma, liver cancer, pancreatic cancer, prostate cancer or sarcoma when n is at least 1, or is capable of achieving diagnostic accuracy having an AUC value greater than approximately 0.800 for detecting ovarian cancer when n is at least 2. I.e. in order to achieve an AUC>˜0.80, any one or more (n=1, 2, 3, . . . ) miRNAs can be taken from the 500-miRNA list in Table 1 in the method to thereby detect each of lung cancer, biliary tract cancer, bladder cancer, colorectal cancer, esophageal cancer, gastric cancer, glioma, liver cancer, pancreatic cancer, prostate cancer or sarcoma, or that for realizing the detection of ovarian cancer with an AUC>˜0.80, the method requires that n is at least two (i.e. any two or more miRNAs be taken from the 500-miRNA list in Table 1).

According to some embodiments, the method is capable of achieving diagnostic accuracy having an AUC value greater than approximately 0.850 for detecting any one of lung cancer, biliary tract cancer, bladder cancer, colorectal cancer, gastric cancer or prostate cancer when n is at least 1, for detecting liver cancer, pancreatic cancer or sarcoma when n is at least 2, for detecting esophageal cancer or glioma when n is at least 3, or for detecting ovarian cancer when n is at least 4.

According to some embodiments, the method is capable of achieving diagnostic accuracy having an AUC value greater than approximately 0.900 for detecting lung cancer, biliary tract cancer, bladder cancer, colorectal cancer, gastric cancer or prostate cancer when n is at least 2. According to some embodiments, the method is capable of achieving diagnostic accuracy having an AUC value greater than approximately 0.950 for detecting lung cancer or gastric cancer or prostate cancer when n is at least 3, for detecting bladder cancer when n is at least 4, or for detecting biliary tract cancer when n is at least 5.

According to some embodiments of the method, in step (2) of calculating the diagnostic index of the biological sample based on the expression profile of the miRNA biomarker set, the diagnostic index is calculated via an unweighted model.

According to some other embodiments of the method, in step (2) of calculating the diagnostic index of the biological sample based on the expression profile of the miRNA biomarker set, the diagnostic index is calculated via a weighted model using weights from one selected from a group consisting of Linear Models for Microarray Data (limma) model, logistic regression model, linear discriminant analysis (LDA) model, conditional logistic regression model, lasso regression model, ridge regression model, random forest, support vector machine, and probit regression model. Further optionally, the diagnostic index is calculated via a weighted model using weights from the limma model.

As used herein, the terms “unweighted model” and “weighted model” are to be understood within the common definition as well appreciated by people skilled in the art. Regarding the term “unweighted model”, it refers to a situation where no weight is applied for each miRNA in the miRNA biomarker set when calculating the diagnostic index. Within the scope of the present disclosure and with reference to formula (I), the phrase “the diagnostic index is calculated via an unweighted model” can be understood to have an equal tᵢ (e.g. tᵢ=1) for any miRNA in the miRNA biomarker set. Regarding the term “weighted model”, it refers to as a situation where a corresponding weight is applied for each miRNA in the miRNA biomarker set when calculating the diagnostic index. Within the scope of the present disclosure and with reference to formula (I), the phrase “the diagnostic index is calculated via a weighted model” can be understood such that for any miRNAi in the miRNA biomarker set, not all tᵢ are equal (i.e. there are at least two miRNAs which have different weights).

Each of the terms “Linear Models for Microarray Data (limma) model” (Ritchie et al. 2015), “logistic regression model” (Venable and Ripley 2002), “linear discriminant analysis (LDA) model” (Venable and Ripley 2002), “conditional logistic regression model” (Venable and Ripley 2002), “lasso regression model” (Tibshirani 1996), “ridge regression model” (Hoerl and Kennard 1970), “random forest” (Ripley 1996), “support vector machine” (Ripley 1996), and “probit regression model” (Venable and Ripley 2002) is substantially a probability-modeling statistical model that models abides by the definition commonly appreciated by people skilled in the field, the details of which can be referenced by the reference included immediately behind.

In order to provide convenience, according to some embodiments, after step (2) and before step (3), the method can further include a normalization step of: obtaining a normalized diagnostic index based on the calculated diagnostic index. Correspondingly, step (3) comprises: classifying the subject as having the cancer if the normalized diagnostic index is equal to or greater than a preset cut-point; or classifying the subject as not having the cancer if otherwise.

Herein, there can be different ways for the normalization step. According to some embodiments, the normalized diagnostic index is calculated based on formula (II):

$$normalized diagnostic index = \frac{diagnostic index - {param}_{location}}{{param}_{scale}} ; \qquad ( II )$$

Herein the param_{location} and param_{scale} are respectively a location parameter and a scale parameter configured to allow the normalized diagnostic index to be within a range no less than a first preset value and no greater than a second preset value.

More specifically, the param_{location} is substantially a location parameter configured to shift the minimum of the normalized diagnostic index to the first preset value, and param_{scale} is substantially a scale parameter configured to scale the maximum of the normalized diagnostic index to the second value. Thus the first preset value and the second preset value are respectively the minimum and maximum in the range of normalized diagnostic index values that have been obtained and calculated from an existing population of subjects known to have and known not to have the cancer, with outliers excluded.

Optionally, multiple settings can be applied. For example, in the existing dataset in EXAMPLE 1 below, where the diagnostic index values are determined to have a range of 600 to 1600 excluding outliers (see), in order to shift the range to be between 0 (i.e. the first preset value) and 10 (i.e. the second present value), the param_{location} and param_{scale} can be respectively set to 600 and 100 so that the final normalized diagnostic index can be no less than 0 and no greater than 10. It is note that this normalization scheme was employed in the below EXAMPLE 1. Alternatively, the param_{location} and param_{scale} can be respectively set to 600 and 1000, so that the final normalized diagnostic index can be set to be no less than 0 and no greater than 1. Further alternatively, the param_{location} and param_{scale} can be respectively set to 600 and 10, so that the final normalized diagnostic index can be set to be no less than 0 and no greater than 100. Further alternatively, the param_{location} and param_{scale} can be respectively set to 350 and 250, so that the final normalized diagnostic index can be set to be no less than 1 and no greater than 5.

According to one specific embodiment of the method, the miRNA biomarker set consists of the top 4 miRNAs listed in Table 1, i.e. hsa-miR-5100, hsa-miR-1343-3p, hsa-miR-1290, and hsa-miR-4787-3p, which are ranked based on the adjusted P value and correspond to SEQ ID NOS: 2-4 respectively. As demonstrated in EXAMPLE 1 below, the method based on this 4-miRNA biomarker set is capable of achieving diagnostic accuracy having a higher AUC value for detecting cancer(s).

Herein, the pre-determined threshold in formula (I) can be set as 1110, such that the method is capable of achieving diagnostic accuracy having a specificity value greater than approximately 0.95. Alternatively, the pre-determined threshold in formula (I) can be set as 1200, such that the method is capable of achieving diagnostic accuracy having a specificity value greater than approximately 0.99.

According to some embodiments, the method is capable of achieving diagnostic accuracy having an AUC value greater than approximately 0.85 for detecting any of the above twelve cancers: lung cancer, biliary tract cancer, bladder cancer, colorectal cancer, esophageal cancer, gastric cancer, glioma cancer, liver cancer, pancreatic cancer, prostate cancer, ovarian cancer, and sarcoma.

According to some embodiments, the method is further capable of achieving diagnostic accuracy having an AUC value greater than approximately 0.95 for detecting any one of eleven out of the twelve cancers including: lung cancer, biliary tract cancer, bladder cancer, colorectal cancer, esophageal cancer, gastric cancer, glioma cancer, liver cancer, ovarian cancer, pancreatic cancer, and prostate cancer.

According to some embodiments, the method is further capable of achieving diagnostic accuracy having an AUC value greater than even approximately 0.99 for detecting any of seven out of the twelve cancers including: lung cancer, biliary tract cancer, bladder cancer, esophageal cancer, gastric cancer, glioma cancer, and prostate cancer.

According to some embodiments, the method is even capable of achieving a diagnostic accuracy having an AUC value greater than approximately 0.999 for detecting lung cancer or gastric cancer.

Depending on different practical needs, the method can optionally be configured to be capable of achieving diagnostic accuracy having different sensitivity and specificity levels.

According to some embodiments, the method is capable of achieving diagnostic accuracy having a sensitivity greater than approximately 68.0% while having a specificity greater than approximately 99.0% for detecting any one of lung cancer, biliary tract cancer, bladder cancer, colorectal cancer, esophageal cancer, gastric cancer, glioma cancer, liver cancer, pancreatic cancer, prostate cancer, ovarian cancer, and sarcoma.

According to some embodiments, the method is capable of achieving diagnostic accuracy having a sensitivity greater than approximately 83.0% while having a specificity greater than approximately 99.0% for detecting lung cancer, biliary tract cancer, bladder cancer, colorectal cancer, esophageal cancer, gastric cancer, glioma cancer, liver cancer, pancreatic cancer, or prostate cancer.

According to some embodiments, the method is capable of achieving diagnostic accuracy having a sensitivity greater than approximately 99.0% and having a specificity greater than approximately 99.0% for detecting lung cancer or gastric cancer.

In embodiments where the normalized diagnostic index is normalized to be between 0 and 10, the pre-set cut-point can optionally be set as 5.1 to thereby allow the method to have a specificity that is greater than approximately 0.95, or optionally can be set as 6.0 to thereby allow the method to have a specificity that is greater than approximately 0.99.

In any embodiment of the method as described above, the biological sample is a liquid biopsy sample selected from a group consisting of a blood sample, a serum sample, a plasma sample, a urine sample (Yun et al. 2012), a saliva sample (Park et al. 2009), and a spatum sample.

In any embodiment of the method as described above, in step (1) of determining an expression profile of an miRNA biomarker set consisting of at least one miRNA from the biological sample, the expression profile of the miRNA biomarker set can optionally be obtained by means of Northern Blotting, microarray analysis, RNA-sequencing, or RNA in-situ hybridization, or can optionally be obtained by means of a nucleic acid amplification procedure, comprising reverse-transcription PCR (RT-PCR), quantitative RT-PCR (qRT-PCR), or digital RT-PCR.

As used herein, each of the above miRNA detection approaches is to be understood within the common definition well-appreciated by people of ordinary skills in the field. More details for implementing these approaches to determine the expression profile of the miRNA biomarker set will be provided below.

In any embodiment of the method as described above, the method optionally further comprises a step of performing an evaluation of the subject, wherein said evaluation comprises a diagnosis of the cancer or a detection of a recurrence of the cancer.

Herein, the phrase “diagnosis of the cancer” is referred to as the detection of the cancer in a subject previously known not to have the cancer, whereas the phrase “recurrence of the cancer” is referred to as the detection of the cancer again in a subject with the cancer who has previously been treated to remove the cancer to become cancer-free.

In any embodiment of the method as described above, the method optionally further comprises a step of administering to the subject a therapeutic regimen when the subject is classified as having the cancer. Herein, a variety of known therapeutic regimens can be administered in the method, which include surgery, radiotherapy, chemotherapy, hormonal therapy, targeted therapy, immunotherapy or the combination thereof. These above therapeutic regimens have been well-established for each different cancer mentioned above.

In any embodiment of the method as described above, the method optionally further comprises a step of performing a diagnostic procedure on the subject when the subject is classified as having the cancer. Herein the diagnostic procedure may optionally comprise physical examination, pathological examination of a biopsy from the subject, immunohistochemistry examination, or imaging examination such as x-rays, computed tomography (CT), ultrasonography, and/or magnetic resonance imaging.

In a second aspect, the present disclosure further provides a kit for detecting a cancer from a biological sample obtained from a subject, which is substantially employed for implementing the method described in the first aspect.

As used herein, and elsewhere in the disclosure as well, the term “kit” is referred to as a collection of articles and/or instructions. An article included in the kit can be a physical entity or a component thereof. Examples of articles that can be included in the kit as disclosed herein can include one or more nucleic acids (e.g. polynucleotides), or one or more device, apparatus or equipment (e.g. a molecular array or microarray that comprises the one or more nucleic acids). An instruction included in the kit can be a description of the specific steps to be performed (e.g. a manual), which can be printed on a physical medium (e.g. paper, card, etc.), on a computer-readable storage medium (e.g. hard disc, compact disc or CD, flash drive, etc.), or even stored in the internet (e.g. in an accessible cloud space), etc.

The kit can comprise at least the following components (1) and (2) (i.e. articles and/or instructions):
Component (1): at least one nucleic acid, each capable of specifically recognizing each miRNA in an miRNA biomarker set to thereby allow an expression profile of the miRNA biomarker set to be obtained from the biological sample. Herein each miRNA of the miRNA biomarker set is from a 500-miRNA set as listed in Table 1.
Component (2): at least one instruction, comprising a first instruction and a second instruction. The first instruction comprises a first sub-instruction for calculating a diagnostic index of the biological sample based on the expression profile of the miRNA biomarker set, wherein the diagnostic index is calculated based on formula:

$$diagnostic index = {\sum }_{i = 1}^{n} {t}_{i} * {miRNA}_{i} ; \qquad ( I )$$

where n is the total number of the at least one miRNA in the miRNA biomarker set and is an integer greater than zero and smaller than or equal to 500; miRNAᵢ is the expression level of i^{th} miRNA in the miRNA biomarker set, i is an integer greater than zero and smaller than or equal to n; and tᵢ is a weight for the i^{th} miRNA. The second instruction is configured for classifying the subject as having the cancer or not, wherein the subject is classified as having the cancer if the calculated diagnostic index is greater than or equal to a pre-determined threshold or as not having the cancer if otherwise.

The kit is configured to be capable of achieving diagnostic accuracy having an AUC value greater than approximately 0.75 for detecting any one of lung cancer, biliary tract cancer, bladder cancer, colorectal cancer, esophageal cancer, gastric cancer, glioma, liver cancer, ovarian cancer, pancreatic cancer, prostate cancer, or sarcoma.

According to some embodiments of the kit, in the first sub-instruction of the first instruction, the diagnostic index is calculated via an unweighted model. According to some other embodiments, the diagnostic index is calculated via a weighted model using weights from one selected from a group consisting of Linear Models for Microarray Data (limma) model, logistic regression model, linear discriminant analysis (LDA) model, conditional logistic regression model, lasso regression model, ridge regression model, random forest, support vector machine, and probit regression model.

Optionally for the kit, the diagnostic index is calculated via a weighted model using weights from the limma model.

In the kit, the first instruction may optionally further comprise a second sub-instruction for obtaining a normalized diagnostic index based on the diagnostic index calculated according to the first sub-instruction, wherein in the second instruction, the subject is classified as having the cancer if the normalized diagnostic index is greater than or equal to a preset cut-point or as not having the cancer if otherwise.

According to some embodiments of the kit, in the second sub-instruction, the normalized diagnostic index is calculated based on formula:

$$normalized diagnostic index = \frac{diagnostic index - {param}_{location}}{{param}_{scale}} ; \qquad ( II )$$

where the param_{location} and param_{scale} are respectively a location parameter and a scale parameter configured to allow the normalized diagnostic index to be within a range no less than a first preset value and no greater than a second preset value.

Optionally in the first instruction, the diagnostic index is calculated via a weighted model using weights from the limma model, and the first preset value is 0, and the second preset value is 10.

According to one preferred embodiment, the miRNA biomarker set consists of the top 4 miRNAs in Table 1, i.e. hsa-miR-5100, hsa-miR-1343-3p, hsa-miR-1290, and hsa-miR-4787-3p, and correspondingly, in component (1) of the kit, the at least one nucleic acid consists of a total of 4 polynucleotides which are respectively capable of specifically hybridizing under a stringent condition to: either (a) polynucleotides respectively comprising or consisting of nucleotide sequences of SEQ ID NOS: 1-4, derivatives thereof, variants thereof each having at least 80% sequence identity, or fragments thereof each comprising 15 or more consecutive nucleotides; or (b) polynucleotides respectively comprising or consisting of nucleotide sequences which are respectively complementary to nucleotide sequences of SEQ ID NOS: 1-4, derivatives thereof, variants thereof each having at least 80% sequence identity, or fragments thereof each comprising 15 or more consecutive nucleotides.

In the kit, in the first sub-instruction of the first instruction in component (2), the diagnostic index can be calculated via an unweighted model, or alternatively via a weighted model using weights from one of the probability-modeling statistical models that have been provided above in the first aspect. Herein according to some embodiments of the kit, the diagnostic index is calculated via a weighted model using weights from the limma model.

According to some embodiments of the kit, the pre-determined threshold can be set as 1110, and the second instruction further indicates that the classification using 1110 as the pre-determined threshold has a specificity >0.95. According to some other embodiments of the kit the pre-determined threshold can be set as 1200, and the second instruction further indicates that such classification using 1200 as the pre-determined threshold has a specificity >0.99.

According to some embodiments of the kit, the first instruction further comprises a second sub-instruction for obtaining a normalized diagnostic index based on the diagnostic index calculated according to the first sub-instruction, and in the second instruction, the subject is classified as having the cancer if the normalized diagnostic index is greater than or equal to a preset cut-point or as not having the cancer if otherwise. The normalization process is substantially identical to the normalization process mentioned above in the first method aspect above, whose description will be skipped in here.

Optionally, the normalized diagnostic index is calculated via a weighted model using weights from the limma model, and the first preset value is 0, and the second preset value is 10. Furthermore, the preset cut-point can be set optionally as 5.1 or 6.0, to thereby allow the classification using the preset cut-point to have a specificity that is >0.95 or >0.99, respectively.

According to different embodiments, the at least one instruction in component (2) in the kit may further comprise a third instruction for performing an evaluation of the subject, wherein said evaluation comprises a diagnosis of the cancer or a detection of a recurrence of the cancer; or may further comprise a fourth instruction for administering to the subject a therapeutic regimen when the subject is classified as having the cancer.

According to some embodiments, the at least one instruction in component (2) in the kit may further comprise a first additional instruction for obtaining the expression profile of the miRNA biomarker set, comprising a procedure for performing Northern Blotting, microarray analysis, RNA-sequencing, or RNA in-situ hybridization by means of the at least one nucleic acid. Herein, the at least one nucleic acid may optionally be arranged on a molecular array.

According to some embodiments, the kit may further comprise at least one set of amplification primers, each set capable of specifically amplifying each of the at least one miRNA in the miRNA biomarker set from the biological sample. As such, the at least one instruction in component (2) in the kit may further comprise a second additional instruction for obtaining the expression profile of the miRNA biomarker set, comprising a procedure for performing reverse-transcription PCR (RT-PCR), quantitative RT-PCR (qRT-PCR), or digital RT-PCR by means of the at least one nucleic acid and the at least one set of amplification primers.

In any embodiment of the kit as described above, the biological sample can be a liquid biopsy sample selected from a group consisting of a blood sample, a serum sample, a plasma sample, a urine sample, a saliva sample, and a spatum sample.

In a third aspect, the present disclosure further provides a system for detecting a cancer in a subject. Herein, the system is substantially a computerized system comprising a collection of hardware (e.g. processor, memory, I/O interface, storage medium, etc.) and software (i.e. computer programs, including operation system software, and specific program software, etc.), which are configured to collaboratively work so as to collectively implement all or some steps of the method as described above in the first aspect. According to some embodiments, the system comprises a processor and a non-transitory storage medium. The non-transitory storage medium is configured to contain a software (i.e. program instructions) for execution by the processor, and the program instructions are configured to cause the processor to execute the various steps of the method according to the various different embodiments of the method that are described above in the first aspect.

In a fourth aspect, the present disclosure further provides a non-transitory storage medium, configured to store computer-executable program instructions which, when executed by a processor, cause the processor to execute the method according to the various different embodiments of the method that are described above in the first aspect.

There can be various different embodiments for the above-mentioned system and non-transitory storage medium regarding to the following elements/features, including: what miRNA components are included in the miRNA biomarker set; whether and how a normalization is performed over the diagnostic index; how the subject is classified as having the cancer or not, what samples can be used for the biological sample, and what detection accuracy level is to be achieved, etc. The specific details for these different embodiments can be referenced to the various embodiments of the method as described in the first aspect, and will be skipped herein for conciseness.

Unless defined elsewhere, the terms as used throughout the disclosure are defined as follows.

In general terms, a “subject” means a mammal such as a primate including a human and a chimpanzee, a pet animal including a dog and a cat, a livestock animal including cattle, a horse, sheep, and a goat, and a rodent including a mouse and a rat. The term “healthy subject” also means such a mammal without the cancer to be detected. It is to be noted that the whole disclosure concerns more specifically human subjects, but can optionally be applied to other non-human mammals as well.

Unless indicated or defined otherwise, the terms or abbreviations such as “nucleic acid”, “nucleotide”, “polynucleotide”, “DNA”, “RNA”, and “miRNA” abide by common use in the art.

As used herein, the term “polynucleotide” is interchangeable with “nucleic acid”, and is referred to as a nucleic acid including all of RNA, DNA, and RNA/DNA (chimera). The DNA includes all of cDNA, genomic DNA, and synthetic DNA. The RNA includes all of total RNA, mRNA, rRNA, miRNA, siRNA, snoRNA, snRNA, non-coding RNA and synthetic RNA.

As used herein, the term “fragment” is a polynucleotide having a nucleotide sequence having a consecutive portion of a polynucleotide and desirably has a length of 15 or more nucleotides, e.g. 15, 16, 17, 18, 19, etc. nucleotides.

As used herein, the term “gene” is intended to include not only RNA and double-stranded DNA but also each single-stranded DNA such as a plus strand (or a sense strand) or a complementary strand (or an antisense strand) constituting the duplex. The gene is not particularly limited by its length. As used herein, the “gene” includes all of double-stranded DNA including human genomic DNA, single-stranded DNA (plus strand) including cDNA, single-stranded DNA having a sequence complementary to the plus strand (complementary strand), miRNA (miRNA), and their fragments, and their transcripts, unless otherwise specified. The “gene” includes not only a “gene” represented by a particular nucleotide sequence (or SEQ ID NO) but “nucleic acids” encoding RNAs having biological functions equivalent to an RNA encoded by the gene, for example, a congener (i.e., a homolog or an ortholog), a variant (e.g., a genetic polymorph), and a derivative. Specific examples of such a “nucleic acid” encoding a congener, a variant, or a derivative can include a “nucleic acid” having a nucleotide sequence hybridizing under stringent conditions described later to a complementary sequence of a nucleotide sequence represented by any of SEQ ID NOs: 1 to 100 or a nucleotide sequence derived from the nucleotide sequence by the replacement of the nucleotide “U” (or “u”) with the nucleotide “T” (or “t”). The “gene” is not particularly limited by its functional region and can contain, for example, an expression control region, a coding region, an exon, or an intron. The “gene” may be contained in a cell or may exist alone after being released into the outside of a cell. Alternatively, the “gene” may be in a state enclosed in a vesicle called exosome.

Within the scope of the whole disclosure, the term “microRNA (miRNA)” is intended to mean a 15- to 25-nucleotide non-coding RNA that is transcribed as an RNA precursor having a hairpin-like structure, cleaved by a dsRNA-cleaving enzyme which has RNase III cleavage activity, integrated into a protein complex called RISC, and involved in the suppression of translation of mRNA, unless otherwise specified. The term “miRNA” as used herein includes not only a “miRNA” represented by a particular nucleotide sequence (or SEQ ID NO) but a precursor of the “miRNA” (pre-miRNA or pri-miRNA), and miRNAs having biological functions equivalent thereto, for example, a congener (i.e., a homolog or an ortholog), a variant (e.g., a genetic polymorph), and a derivative. Such a precursor, a congener, a variant, or a derivative can be specifically identified using miRBase Release 20 (Kozomara and Griffiths-Jones, 2010), and examples thereof can include an “miRNA” having a nucleotide sequence hybridizing under stringent conditions described later to a complementary sequence of any particular nucleotide sequence represented by any of SEQ ID NOS: 1 to 100. The term “miRNA” as used herein may be a gene product of a miRNA gene. Such a gene product includes a mature miRNA (e.g., a 15- to 25-nucleotide or 19- to 25-nucleotide non-coding RNA involved in the suppression of translation of mRNA as described above) or a miRNA precursor (e.g., pre-miRNA or pri-miRNA).

As used herein, the term “probe” includes a polynucleotide that is used for specifically detecting an RNA resulting from the expression of a gene or a polynucleotide derived from the RNA, and/or a polynucleotide complementary thereto.

As used herein, the term “primer”, or “amplification primers” includes a polynucleotide that specifically recognizes and amplifies an RNA resulting from the expression of a gene or a polynucleotide derived from the RNA, and/or a polynucleotide complementary thereto.

In this context, the complementary polynucleotide (complementary strand or reverse strand) means a polynucleotide in a complementary base relationship based on A:T (U) and G:C base pairs with the full-length sequence of a polynucleotide consisting of a nucleotide sequence defined by any of SEQ ID NOs: 1 to 100 or a nucleotide sequence derived from the nucleotide sequence by the replacement of the nucleotide “U” (or “u”) with the nucleotide “T” (or “t”), or a partial sequence thereof (here, this full-length or partial sequence is referred to as a plus strand for the sake of convenience). However, such a complementary strand is not limited to a sequence completely complementary to the nucleotide sequence of the target plus strand and may have a complementary relationship to an extent that permits hybridization under stringent conditions to the target plus strand.

As used herein, the term “stringent conditions” refers to conditions under which a nucleic acid probe hybridizes to its target sequence to a larger extent (e.g., a measurement value equal to or larger than a mean of background measurement values+a standard deviation of the background measurement values×2) than that for other sequences. The stringent conditions are dependent on a sequence and differ depending on an environment where hybridization is performed. A target sequence complementary 100% to the nucleic acid probe can be identified by controlling the stringency of hybridization and/or washing conditions. Specific examples of the “stringent conditions” will be mentioned later.

As used herein, the term “variant” means, in the case of a nucleic acid, a natural variant attributed to polymorphism, mutation, or the like; a variant containing the deletion, substitution, addition, or insertion of 1, 2, or 3 or more nucleotides in a nucleotide sequence represented by any of SEQ ID NOs: 1 to 100 or a nucleotide sequence derived from the nucleotide sequence by the replacement of the nucleotide “U” (or “u”) with the nucleotide “T” (or “t”), or a partial sequence thereof; a variant containing the deletion, substitution, addition, or insertion of 1 or 2 or more nucleotides in a nucleotide sequence of a premature miRNA of a sequence represented by any of SEQ ID NOs: 1 to 100 or a nucleotide sequence derived from the nucleotide sequence by the replacement of the nucleotide “U” (or “u”) with the nucleotide “T” (or “t”), or a partial sequence thereof; a variant that exhibits % identity of approximately 90% or higher, approximately 95% or higher, approximately 97% or higher, approximately 98% or higher, approximately 99% or higher to each of these nucleotide sequences or the partial sequences thereof; or a nucleic acid hybridizing under the stringent conditions defined above to a polynucleotide or an oligonucleotide comprising each of these nucleotide sequences or the partial sequences thereof. A variant can be prepared by use of a well-known technique such as site-directed mutagenesis or PCR-based mutagenesis.

The term “percent (%) identity” can be determined with or without an introduced gap, using a protein or gene search system based on BLAST or FASTA described above (Zhang et al., 2000; Altschul et al. 1990; Pearson et al. 1988).

The term “derivative” is meant to include a modified nucleic acid, for example, a derivative labeled with a fluorophore or the like, a derivative containing a modified nucleotide (e.g., a nucleotide containing a group such as halogen, alkyl such as methyl, alkoxy such as methoxy, thio, or carboxymethyl, and a nucleotide that has undergone base rearrangement, double bond saturation, deamination, replacement of an oxygen molecule with a sulfur atom, etc.), PNA (peptide nucleic acid; Nielsen et al. 1991), and LNA (locked nucleic acid; Obika et al. 1998) without any limitation.

The “nucleic acid” capable of specifically binding to a polynucleotide selected from the miRNAs described above is a synthesized or prepared nucleic acid and specifically includes a “nucleic acid probe” or a “primer”. The “nucleic acid” is utilized directly or indirectly for detecting the presence or absence of cancer in a subject, for diagnosing the severity, the degree of amelioration, or the therapeutic sensitivity of cancer, or for screening for a candidate substance useful in the prevention, amelioration, or treatment of cancer. The “nucleic acid” includes a nucleotide, an oligonucleotide, and a polynucleotide capable of specifically recognizing and binding to a transcript represented by any of SEQ ID NOs: 1 to 100, or a synthetic cDNA nucleic acid thereof in vivo, particularly, in a sample such as a body fluid (e.g., blood or urine), in relation to the development of cancer. The nucleotide, the oligonucleotide, and the polynucleotide can be effectively used as probes for detecting the aforementioned gene expressed in vivo, in tissues, in cells, or the like on the basis of the properties described above, or as primers for amplifying the aforementioned gene expressed in vivo.

The term “detection” as used herein is interchangeable with the term “examination”, “measurement”, or “detection or decision support”. As used herein, the term “evaluation” is meant to include diagnosis or evaluation support on the basis of examination results or measurement results.

As used within the scope of the disclosure, each of the terms “P-value”, “accuracy”, “AUC”, “sensitivity”, and “specificity” is generally to be understood to have the common definition that is well appreciated by people skilled in the art, and is specifically defined as follows: The term “P-value” or “P”, is considered to be exchangeable with “p-value” or “p”, and refers to a probability at which a more extreme statistic than that actually calculated from data under a null hypothesis is observed in a statistical test. Thus, smaller “P” or “P value” means more significant difference between subjects to be compared.

The term “AUC” means area under the curve of a Receiver Operating Characteristic curve. The term “accuracy” means a value of (the number of true positives+the number of true negatives)/(the total number of cases). The accuracy indicates the ratio of samples that were correctly identified to all samples and serves as a primary index to evaluate detection performance.

As used herein, the term “sensitivity” means a value of (the number of true positives)/(the number of true positives+the number of false negatives). High sensitivity allows cancer to be detected, leading to clinical treatment interventions.

As used herein, the term “specificity” means a value of (the number of true negatives)/(the number of true negatives+the number of false positives). High specificity prevents needless extra examination for healthy subjects misjudged as being cancer patients, leading to reduction in burden on patients and reduction in medical expense.

Unless specified elsewhere, the following summarizes the available technologies that can be used for the determination of the expression profile of the miRNA biomarker set.

It is to be noted that determination of the expression profile of the miRNA biomarker set substantially includes the determination of the expression level of each and every miRNA contained in the miRNA biomarker set. Preferably, expression levels for all of the miRNA contained in the miRNA biomarker set can be determined simultaneously in one single experiment that is well-controlled. Yet optionally, it is possible that expression levels of these miRNAs are determined in more than one experiment and by different experiment procedure.

As used herein, measuring or detecting the expression of any of the miRNAs contained in the miRNA biomarker set comprises measuring or detecting any nucleic acid transcript corresponding to the miRNA.

Typically, expression can be detected or measured on the basis of miRNA or corresponding reverse transcribed cDNA levels. Any quantitative or qualitative method for measuring RNA levels, or cDNA levels can be used. Suitable methods of detecting or measuring miRNA or cDNA levels include, for example, Northern Blotting, microarray analysis, RNA-sequencing, RNA in-situ hybridization, or a nucleic acid amplification procedure, such as reverse-transcription PCR (RT-PCR) or real-time RT-PCR, also known as quantitative RT-PCR (qRT-PCR), or digital RT-PCR. Such methods are well known in the art (see e.g., Green and Sambrook et al. 2012). Other techniques include digital, multiplexed analysis of gene expression, such as the nCounter® (NanoString Technologies, Seattle, WA) gene expression assays, which are further described in US20100112710 and US20100047924.

Detecting a nucleic acid of interest generally involves hybridization between a target (e.g. miRNA or cDNA) and a probe. Sequences of the miRNAs used in various cancer gene expression profiles are known. Therefore, one of skills in the art can readily design hybridization probes for detecting those miRNAs (see e.g., Green and Sambrook et al. 2012). For example, polynucleotide probes that specifically bind to the miRNA transcripts described herein (or cDNA synthesized therefrom) can be created using the nucleic acid sequences of the miRNA or cDNA targets themselves by routine techniques (e.g., PCR or synthesis). As used herein, the term “probe” means a part or portion of a polynucleotide sequence comprising about 10 or more contiguous nucleotides, about 15 or more contiguous nucleotides, about 20 or more contiguous nucleotides. In certain embodiments, the polynucleotide probes will comprise 10 or more nucleic acids, 15 or more nucleic acids, or 20 or more nucleic acids. In order to confer sufficient specificity, the probe may have a sequence identity to a complement of the target sequence of about 90% or more, such as about 95% or more (e.g., about 98% or more or about 99% or more) as determined, for example, using the well-known Basic Local Alignment Search Tool (BLAST) algorithm (available through the National Center for Biotechnology Information (NCBI), Bethesda, Md.).

Each probe may be substantially specific for its target, to avoid any cross hybridization and false positives. An alternative to using specific probes is to use specific reagents when deriving materials from transcripts (e.g., during cDNA production, or using target-specific primers during amplification). In both cases specificity can be achieved by hybridization to portions of the targets that are substantially unique within the group of miRNAs being analyzed, for example hybridization to the poly A tail would not provide specificity. If a target has multiple splice variants, it is possible to design a hybridization reagent that recognizes a region common to each variant and/or to use more than one reagent, each of which may recognize one or more variants.

Stringency of hybridization reactions is readily determinable by one of ordinary skill in the art, and generally is an empirical calculation dependent upon probe length, washing temperature, and salt concentration. In general, longer probes may require higher temperatures for proper annealing, while shorter probes may require lower temperatures. Hybridization generally depends on the ability of denatured nucleic acid sequences to reanneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so.

“Stringent conditions” or “high stringency conditions,” as defined herein, are identified by, but not limited to, those that: (1) use low ionic strength and high temperature for washing, for example 0.015 M sodium chloride/0.0015 M sodium citrate/0.1% sodium dodecyl sulfate at 50° C.; (2) use during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) use 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash of 0.1×SSC containing EDTA at 55° C. “Moderately stringent conditions” are described by, but not limited to, those in Sambrook et al. 1989, and include the use of washing solution and hybridization conditions (e.g., temperature, ionic strength and % SDS) less stringent than those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/mL denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

In certain embodiments, microarray analysis, Northern blot, RNA in-situ hybridization, or a PCR-based method is used. In this respect, measuring the expression of the foregoing miRNAs in a biological sample can comprise, for instance, contacting a sample containing or suspected of containing cancer cells with polynucleotide probes specific to the miRNAs of interest, or with primers designed to amplify a portion of the miRNAs of interest, and detecting binding of the probes to the nucleic acid targets or amplification of the nucleic acids, respectively. Detailed protocols for designing PCR primers are known in the art (see e.g., Green and Sambrook et al. 2012). In certain embodiments, miRNAs obtained from a sample may be subjected to qRT-PCR. Reverse transcription may occur by any methods known in the art, such as through the use of an Omniscript RT Kit (Qiagen). The resultant cDNA may then be amplified by any amplification technique known in the art. miRNA expression may then be analyzed through the use of, for example, control samples as described below. As described herein, the over- or under-expression of miRNAs relative to controls may be measured to determine a miRNA expression profile for an individual biological sample. Similarly, detailed protocols for preparing and using microarrays to analyze miRNA expression are known in the art and described herein.

As used herein, RNA-sequencing (RNA-seq), also called Whole Transcriptome Shotgun Sequencing, refers to any of a variety of high-throughput sequencing techniques used to detect the presence and quantity of RNA transcripts in real time. See Wang, Z., M. Gerstein, and M. Snyder, RNA-Seq: a revolutionary tool for transcriptomics, NAT REV GENET, 2009. 10(1): p. 57-63. RNA-seq can be used to reveal a snapshot of a sample's miRNAs from a genome at a given moment in time. In certain embodiments, miRNA is converted to cDNA fragments via reverse transcription prior to sequencing, and, in certain embodiments, miRNA can be directly sequenced without conversion to cDNA. Adaptors may be attached to the 5′ and/or 3′ ends of the miRNAs, and the miRNA or cDNA may optionally be amplified, for example by PCR. The fragments are then sequenced using high-throughput sequencing technology, such as, for example, those available from Roche (e.g., the 454 platform), Illumina, Inc., and Applied Biosystem (e.g., the SOLID system).

### BRIEF DESCRIPTION OF THE DRAWINGS

FIGS. 1A-1C show a case flow diagram for lung cancer dataset (FIG. 1A, split into a discovery and a validation set) and for ovarian, liver and bladder cancer datasets (FIG. 1B, combined into a single validation dataset after removing redundant samples), and summarize the patient and tumor characteristics of patients with lung, bladder, ovarian, and liver cancers and demographic information of the corresponding controls (FIG. 1C);

FIGS. 2A-2G show the development and validation of the 4-miRNA diagnostic model in the lung cancer data set, with FIG. 2A showing determination of the optimal number (dotted line) of miRNAs for the diagnostic model by 10-fold cross validation in the discovery set; FIG. 2B showing ROC analysis in the discovery set; FIG. 2C showing distribution of normalized diagnostic index in the discovery set; FIG. 2D showing ROC analysis in the validation set; FIG. 2E showing distribution of normalized diagnostic index in the validation set; FIG. 2F showing comparison of normalized diagnostic index of paired serum samples (pre- vs. post-surgery) of 180 lung cancer patients; and FIG. 2G showing distribution of normalized diagnostic index in the clinical subsets of the validation set. Dotted horizontal lines represent the cut-point for the normalized diagnostic index of our model. The percentages shown in the graph were sensitivities in each cancer subgroup.

FIGS. 3A and 3B show the performance of 4-miRNA diagnostic model in the datasets of additional cancers, with FIG. 3A showing ROC analysis, and FIG. 3B showing distribution of normalized diagnostic index the 4-miRNA model. The percentages shown in the graph were sensitivities of each cancer type and specificity of non-cancer controls;

FIGS. 4A and 4B show the ROC analysis and distribution of normalized diagnostic index across age and gender groups in the lung cancer dataset;

FIG. 5 shows the block diagram of the computerized system used for implementing the method as provided in the disclosure;

FIGS. 6A-6Q respectively show the performance of any number (1, 2, 3, . . . , 500) of miRNAs randomly selected from the top 500-miRNAs identified by the limma analysis in the lung cancer training set in detecting multiple cancer types beyond lung cancer.

### DETAILED DESCRIPTION

The present disclosure provides an approach, comprising a method, a kit and a computerized system, that is capable of accurately and reliably detecting one or multiple human cancers for a subject based on the expression profile of an miRNA biomarker set consisting of at least one miRNA, each from a 500-miRNA set as listed in Table 1.

In the first aspect of this section, a detection method capable of achieving diagnostic accuracy having an AUC value greater than approximately 0.75 is provided, which substantially includes the following three steps:
Step (1): determining the expression profile of an miRNA biomarker set from the biological sample;
Step (2): calculating a diagnostic index of the biological sample based on the expression profile of the miRNA biomarker set. The diagnostic index is calculated based on:

$$diagnostic index = {\sum }_{i = 1}^{n} {t}_{i} * {miRNA}_{i} ; \qquad ( I )$$

where n is the total number of miRNAs in the miRNA biomarker set and and is an integer greater than zero and smaller than or equal to 500; miRNAᵢ is the expression level of the i^{th} miRNA in the miRNA biomarker set, where i is an integer greater than zero and smaller than or equal to n; and tᵢ is a weight for the i^{th} miRNA; and
Step (3): classifying the subject as having the cancer or not based on the value of the calculated diagnostic index. If the calculated diagnostic index is greater than or equal to a pre-determined threshold, the subject is classified as having the cancer; or if otherwise the subject is classified as not having the cancer.

Herein, the miRNA biomarker set includes any one or a combination of the miRNAs listed in Table 1 (see EXAMPLE 1 and EXAMPLE 2). According to different embodiments, the miRNA biomarker set may include miRNA(s) from the top 1-500 miRNAs, or alternatively may further include miRNA(s) from the top 1-250 miRNAs, or alternatively may further include miRNA(s) from the top 1-100 miRNAs, or alternatively may further include miRNAs from the top 1-50 miRNAs, or alternatively may further include miRNAs from the top 1-20 miRNAs, or alternatively may further include miRNA(s) from the top 2-4 miRNAs, in Table 1.

Preferably, the miRNA biomarker set consists of the top 4 miRNAs (i.e. hsa-miR-5100, hsa-miR-1343-3p, hsa-miR-1290, and hsa-miR-4787-3p). Herein, depending on different embodiments, there can be different AUC cut-off levels (e.g. 0.780, 0.850, 0.950, 0.990, and 0.999), or different sensitivity-specificity levels (e.g. 68%-99%, 68%-99%, 83%-99%, and 99%-99%), at least at which the method is capable of accurately detecting certain cancer types. For example, the method can accurately detect lung cancer and gastric cancer at the AUC>0.999, and/or at a sensitivity >99.0% and having a specificity >99.0%.

There can be different ways to calculate the diagnostic index based on formula (I). Optionally, the calculation can be based on an unweighted model or on a weighted model. Under the latter situation, different models (e.g. limma model, logistic regression model, etc.) may optionally be applied for obtaining the weights for the miRNAs in the miRNA biomarker set.

Preferably, the diagnostic index is calculated via a weighted model using weights from the limma model. Herein, in step (3) of the method, the pre-determined threshold can be set as 1110 to thereby allow the method to have a specificity >0.95; or optionally, the pre-determined threshold can be set as 1200 to thereby allow the method to have a specificity >0.99.

Optionally the diagnostic index calculated in step (2) can further undergo a normalization process, and the step (3) can determine the cancer classification based on whether the normalized diagnostic index is no less than or greater than a preset cut-point.

It is noted that selection of the normalization process is arbitrary. According to some embodiments, the normalization process can be based on formula:

$$normalized diagnostic index = \frac{diagnostic index - {param}_{location}}{{param}_{scale}} ; \qquad ( II )$$

where the param_{location} and param_{scale} are respectively a location parameter and a scale parameter configured to allow the normalized diagnostic index to be within a range no less than a first preset value and no greater than a second preset value.

Herein, optionally, the param_{location} and param_{scale} can be selected as 600 and 1000 respectively to thereby allow the normalized diagnostic index to be between 0 and 10, and under such normalization, the preset cut-point can be set as 5.1 to give a specificity >0.95 or as 6.0 to give a specificity >0.99.

In the method, the biological sample can advantageously be a liquid biopsy sample such as a blood sample, a serum sample, a plasma sample, a urine sample, a saliva sample, or a spatum sample, etc. Determination of the expression profile of the miRNA biomarker set can be realized by means of a variety of probe-based approaches including Northern Blotting, microarray analysis, RNA-sequencing, or RNA in-situ hybridization, or by means of a variety of amplification-dependent approaches including reverse-transcription PCR (RT-PCR), quantitative RT-PCR (qRT-PCR), or digital RT-PCR.

Optionally, the method may further comprise a step of performing an evaluation of the subject, so as to determine if the subject is diagnosed as having the cancer (if the subject is absent of cancer before) or if the subject has recurrence of the cancer (if the subject has been treated to remove, or be free of, the cancer before). For such a purpose, the evaluation may further include physical examination, pathological examination of a biopsy from the subject, immunohistochemistry examination, or imaging examination including x-rays, computed tomography (CT), ultrasonography, magnetic resonance imaging, etc.

Further optionally, the method may further comprise a step of administering to the subject a therapeutic regimen, such as surgery, radiotherapy, chemotherapy, hormonal therapy, targeted therapy, immunotherapy or the combination thereof, when the subject is classified as having the cancer.

In the second aspect, a kit that can be employed to specifically implement the various steps of the method according to the different embodiments as described above in the first aspect of this section is further provided.

The kit substantially include certain articles (i.e. component (1), including one or more nucleic acids that can specifically recognize each miRNA in the miRNA biomarker set, and optionally one or more amplification primers) that can be used to determine the expression profile of the miRNA biomarker set and certain instructions (i.e. component (2)) for calculating the diagnostic index and for cancer classification.

Depending on the miRNAs included in the miRNA biomarker set, each of the nucleic acids in component (1) may comprise a polynucleotide capable of specifically hybridizing under a stringent condition to (a) a polynucleotide comprising or consisting of a nucleotide sequence as set forth in SEQ ID NOS: 1-500, 1-250, 1-100, 1-50, 1-20 or 1-4, a derivative thereof, a variant thereof having at least 80% sequence identity, or a fragment thereof comprising 15 or more consecutive nucleotides; or (b) a polynucleotide comprising or consisting of a nucleotide sequence complementary to a nucleotide sequence of SEQ ID NOS: 1-500, 1-250, 1-100, 1-50, 1-20 or 1-4, a derivative thereof, a variant thereof having at least 80% sequence identity, or a fragment thereof comprising 15 or more consecutive nucleotides.

There can be various different embodiments for the kit regarding to the following elements/features, including: what miRNA components are included in the miRNA biomarker set; whether and how a normalization is performed over the diagnostic index; how the subject is classified as having the cancer or not, what samples can be used for the biological sample, and what detection accuracy level is to be achieved, etc. The specific details for these different embodiments can be referenced to the various embodiments of the method as described above, and will be skipped herein for conciseness.

In the third aspect of this section, a computerized solution is further provided, which substantially serves, in a computerized and automatic manner, to implement the various steps of the method as described above in the first aspect of this section.

Such a computerized solution may be applied in a situation where the implementation of the various steps (1)-(3) of the method described above is to be automated by running a software program comprising program instructions in a computer, which brings about advantages such as high efficiency and great convenience.

Specifically, such a computerized solution may include a computerized system or computer system, which comprises a processor (i.e. controller) and a computer-readable non-transitory storage medium that is communicatively coupled to the processor. The computer-readable non-transitory storage medium is configured to store program instructions that are executable by the processor, thereby causing the processor to execute the various different steps in the method as described above, including:
Step (1): determining the expression profile of the miRNA biomarker set;
Step (2): calculating a diagnostic index of the biological sample based on the expression profile of the miRNA biomarker set and according to formula (I); and
Step (3): classifying the subject as having the cancer or not based on the value of the calculated diagnostic index.

As used herein, the “processor” is interpreted to be exchangeable with “central controller” or “central computing unit (CPU)”, and can be deemed to be a single core or multi core processor, or a plurality of processors for parallel processing. The term “non-transitory,” as used herein, is intended to describe a tangible computer-readable storage medium excluding propagating electromagnetic signals, but are not intended to otherwise limit the type of physical computer-readable storage device that is encompassed by the phrase. Examples may include any tangible or non-transitory storage media or memory media such as electronic, magnetic, or optical media (e.g., disk or CD/DVD-ROM), or non-volatile memory storage (e.g., “flash” memory), etc.

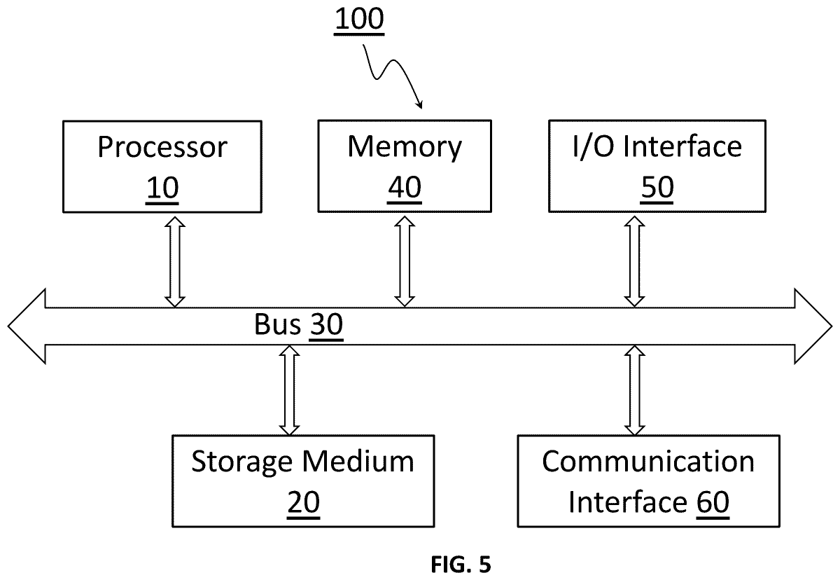

As illustrated in FIG. 5, the system 100 can, in addition to the processor 10 and the computer-readable non-transitory storage medium 20, further comprise a bus 30, a memory 40, an I/O interface 50, and a communication interface 60. The processor 10, the storage medium 20, the memory 40, the I/O interface 50 and the communication interface 60 are all communicatively coupled with one another through the bus 30.

The storage medium 20 stores computer-executable program instructions which, when executed by the processor 10, cause the processor 10 to execute steps (1)-(3) of the method as described above. The memory 40 is configured to transiently store the program instructions obtained from the storage medium 20, and the processor 10 is configured to execute the program instructions transiently stored in the memory 40. The I/O interface 50 allows an input/output between the system 100 and a user, realizing the control of the system 100. The communication interface 60 can allow the system 100 to be communicatively connected to another computing device to exchange data. It is to be noted that these computer hardware components can be locally arranged, or can be remotely arranged via a network, such as an intranet, an internet, or a cloud.

In the following, two examples are provided to illustrate the inventions as described above in the various aspects of the disclosure.

### Example 1

In this example, development and validation of a circulating cell-free miRNA-based diagnostic signature for MCED is provided by utilizing four large miRNA microarray datasets, all based on a standardized microarray platform.

### 2. Materials and Methods

### 2.1. Study Design

Four microarray datasets totaling 7536 unique participants including 3604 cancer patients and 3932 non-cancer controls were included in the current analysis, all derived from studies originating from a Japanese nationwide research project “Development and Diagnostic Technology for Detection of miRNA in Body Fluids” designed to characterize serum miRNAs in over 50,000 participants across 13 cancer types using a standardized microarray platform (Asakura et al. 2020; Yokoi et al. 2018; Usuba et al. 2019, Yamamoto et al. 2020). The four datasets were originally assembled to develop diagnostic signatures for lung (GSE137140), ovarian (GSE106817), liver (GSE113740), and bladder (GSE113486) cancers, respectively.

The lung cancer dataset has the largest sample size for a single cancer type (n=1566) and non-cancer controls (n=2178). The original lung cancer study established a 2-miRNA diagnostic model (referred to as the “original 2-miRNA model” in this study) with high sensitivity and specificity for the detection of lung cancer (Asakura et al. 2020). The objective of the current study was initially set to use this dataset to develop and validate a new diagnostic model that may out-perform the original 2-miRNA model for lung cancer detection. As datasets for additional cancer types were identified, the new model was evaluated for performance to detect other cancers.

### 2.2. Participants and Serum Samples

Serum sample collection has been previously described in the original publications (Asakura et al. 2020; Yokoi et al. 2018; Usuba et al. 2019, Yamamoto et al. 2020). Briefly, serum samples were collected from cancer patients who were referred or admitted to the National Cancer Center Hospital (NCCH) between 2008 to 2016 prior to surgical operation, and stored at 4° C. for one week before being stored at −20° C. until further use. Cancer patients who were treated with preoperative chemotherapy and radiotherapy prior to serum collection were excluded. The serum samples for non-cancer controls who had no history of cancer and no hospitalization during the previous 3 months were collected along with routine blood tests from outpatient departments of three sources: NCCH, National Center for Geriatrics and Gerontology (NCGG) Biobank, and Yokohama Minoru Clinic (YMC). Serums collected from NCCH were stored in the same way as the cancer patients, while those from NCGG and YMC were stored at −80° C. till use. The original studies were approved by the NCCH Institutional Review Board, the Ethics and Conflict of Interest Committee of the NCGG, and the Research Ethics Committee of Medical Corporation Shintokai YMC. Written informed consent was obtained from each participant.

### 2.3. miRNA Microarray Expression Analysis

Details about microarray analysis were described in the original publications (Asakura et al. 2020; Yokoi et al. 2018; Usuba et al. 2019, Yamamoto et al. 2020). Briefly, total RNA was extracted from 300 μL serum, labeled by 3DGene® miRNA Labeling kit and hybridized to 3D-Gene® Human miRNA Oligo Chip (Toray Industries, Kanagawa, Japan) designed to investigate 2588 miRNA sequences registered in miRBase release 21. The following low-quality samples were excluded: coefficient of variation of negative control probes >0.15; and number of flagged probes identified by 3D-Gene® Scanner as “uneven spot images”>10. The presence of a miRNA was determined when signal intensity was greater than mean plus two times standard deviation of the negative control signals, and in using the negative control signals the top and bottom 5% of the ranked signal intensities were removed. Background subtraction was performed by subtracting the mean signal of negative control signals (after removing top and bottom 5% as ranked by signal intensities) from the miRNA signal. Normalization across microarrays was achieved by calibrating according to three pre-selected internal control miRNAs (miR-149-3p, miR-2861, and miR-4463).

### 2.4. Diagnostic Model Development

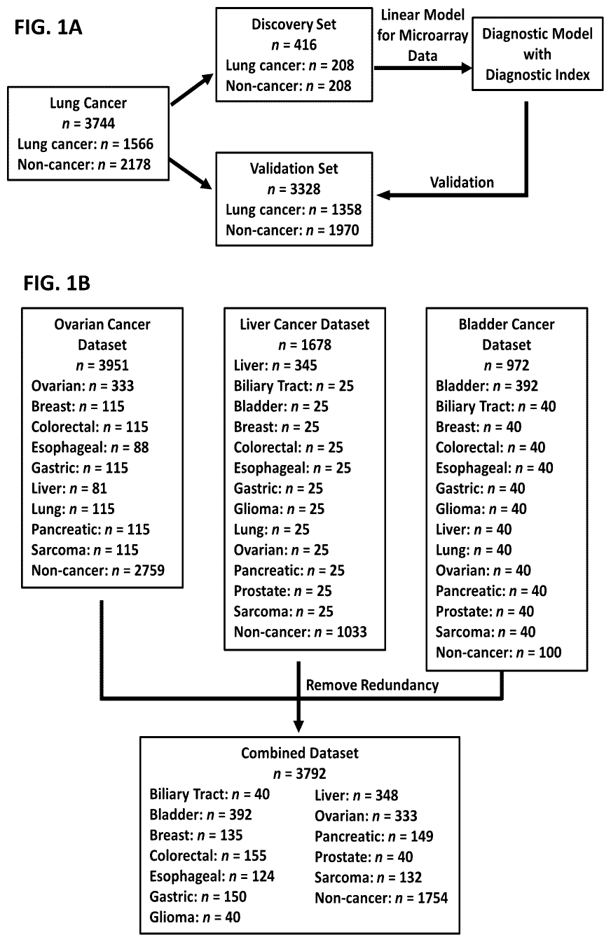

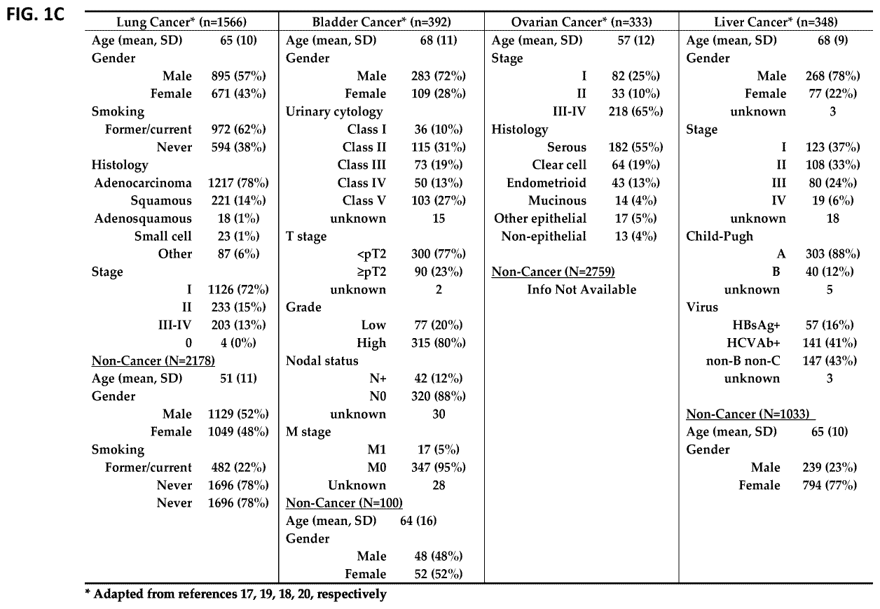

Patients in the lung cancer dataset were divided into the same discovery and validation sets as in the original publication (FIG. 1A) (Asakura et al. 2020), because (1) the discovery set was selected by the original authors to be balanced between cancer and non-cancer with respect to age, sex, and smoking history; (2) 50% of non-cancer participants in the discovery set were from NCCH with the same serum storage condition as cancer patients to minimize potential bias in miRNA candidates selection; (3) Using the same discovery and validation sets allows direct performance comparison of the new diagnostic model with the original 2-miRNA model. As the diagnostic model was developed from the lung cancer discovery set, after its validation in the lung cancer validation set, we further tested its ability as a multi-cancer diagnostic model in a combined dataset of other additional cancer types that were not used in the model development.

Linear Model for Microarray Data (limma) (Ritchie et al. 2015) was performed in the discovery set to evaluate the statistical significance of differential miRNA expression between lung cancer vs. non-cancer. Ten-fold cross validation in the discovery set, based on the area under the curve (AUC) of the Receiver's Operating Characteristics (ROC) curve analysis, was performed to determine the optimal number of miRNAs for the best diagnostic model. A diagnostic index was calculated as a linear sum of miRNA expression levels weighted by limma statistics. The cut-point for the diagnostic index was chosen to ensure no misclassification of non-cancer controls in the discovery set to minimize false positives as the diagnostic model may potentially be used as a screening test in the at-risk general public.

### 2.5. Statistical Analysis

The diagnostic performance for identifying cancer vs. non-cancer was determined by AUC of the ROC curve analysis, sensitivity, and specificity. Comparing AUC of two ROC curves was done with roc.test function with bootstrapping method from pROC package. Comparing paired sensitivities for the lung cancer clinical subsets of paired pre- vs. post-surgical samples was performed by McNemar test. limma analysis was carried out using Bioconductor package limma (The Bioconductor Open Source Software For Bioinformatics (accessed on Aug. 27, 2020). All statistical analysis was performed using R version 4.0.5 (The R Project for Statistical Computing (accessed on Jul. 15, 2020)).

### 3. Results

### 3.1. Participants and Datasets

The lung cancer dataset included 1566 lung cancer patients and 2178 non-cancer controls (FIG. 1A) (Asakura et al. 2020). The ovarian cancer dataset consisted of 333 ovarian cancer patients and 2759 non-cancer controls, as well as patients with breast, colorectal, esophageal, gastric, liver, lung, pancreatic, and sarcoma cancers (FIG. 1B) (Yokoi et al. 2018). The liver and bladder cancer datasets included 345 liver cancer/1033 non-cancer and 392 bladder cancer/100 non-cancer participants, respectively, in addition to patients with biliary tract, breast, colorectal, esophageal, gastric, glioma, lung, ovarian, pancreatic, prostate, and sarcoma cancers (FIG. 1B) (Usuba et al. 2019, Yamamoto et al. 2020). With the lung cancer dataset left intact, redundant samples within the other three datasets that showed correlations either among themselves or with samples in the lung cancer dataset being greater than 0.99 were removed. Then, the unique samples from the ovarian, liver, and bladder cancer datasets were then combined into a single non-lung cancer dataset with a total of 3792 samples, including 2038 cancer patients across 12 cancer types and 1754 non-cancer controls (FIG. 1B).

The lung cancer dataset was divided into the same discovery set (n=416) and validation set (n=3328) as the original study (FIG. 1A). The discovery set included 208 lung cancer patients and 208 non-cancer controls, matched by age, sex, and smoking status (Asakura et al. 2020). The validation set included 1358 lung cancer patients and 1970 non-cancer controls. The patients with lung cancer included 57% male, 62% former or current smokers, 78% adenocarcinoma, 14% squamous carcinoma, 72% stage I, 15% stage II, and 13% stage III (FIG. 1C).

The 392 bladder cancer patients were of mean age 68 y, 72% male, 5% metastatic, 12% nodal positive, 77% T2 or below, and 80% high grade (FIG. 1C). The 333 ovarian cancer patients were of mean age 57 y, 25% stage I, 10% stage II, 55% serous, 19% clear cell, and 13% endometrioid histology (FIG. 1C). The 348 liver cancer patients were of mean age 68 y, 78% male, 37% stage I, and 33% stage II (FIG. 1C). No detailed demographic information and tumor characteristics for the other cancers were provided by the original studies.

TABLE 1
Top 500 differentially expressed miRNAs from the lung cancer discovery set.

| | miRBase Accession | Log Fold | Adjusted | |
|---|---|---|---|---|
| miRNA name | ID | Change | P-value | SEQ ID NO. |
| hsa-miR-5100 | MIMAT0022259 | 3.931 | 9.99E−176 | SEQ ID NO: 1 |
| hsa-miR-1343-3p | MIMAT0019776 | 2.609 | 5.83E−94 | SEQ ID NO: 2 |
| hsa-miR-1290 | MIMAT0005880 | 6.538 | 2.22E−87 | SEQ ID NO: 3 |
| hsa-miR-4787-3p | MIMAT0019957 | 1.854 | 6.81E−67 | SEQ ID NO: 4 |
| hsa-miR-6877-5p | MIMAT0027654 | 1.364 | 1.37E−63 | SEQ ID NO: 5 |
| hsa-miR-17-3p | MIMAT0000071 | 4.088 | 1.66E−62 | SEQ ID NO: 6 |
| hsa-miR-6765-5p | MIMAT0027430 | −0.688 | 5.09E−62 | SEQ ID NO: 7 |
| hsa-miR-1268b | MIMAT0018925 | −0.618 | 1.45E−61 | SEQ ID NO: 8 |
| hsa-miR-4258 | MIMAT0016879 | 1.777 | 6.45E−59 | SEQ ID NO: 9 |
| hsa-miR-451a | MIMAT0001631 | 5.008 | 3.71E−58 | SEQ ID NO: 10 |
| hsa-miR-1228-5p | MIMAT0005582 | −0.780 | 1.01E−57 | SEQ ID NO: 11 |
| hsa-miR-8073 | MIMAT0031000 | 2.087 | 6.42E−55 | SEQ ID NO: 12 |
| hsa-miR-4454 | MIMAT0018976 | 1.661 | 3.91E−51 | SEQ ID NO: 13 |
| hsa-miR-187-5p | MIMAT0004561 | 1.044 | 1.90E−50 | SEQ ID NO: 14 |
| hsa-miR-4286 | MIMAT0016916 | 1.590 | 1.05E−49 | SEQ ID NO: 15 |
| hsa-miR-6746-5p | MIMAT0027392 | 1.346 | 1.53E−49 | SEQ ID NO: 16 |
| hsa-miR-663b | MIMAT0005867 | 1.201 | 9.31E−49 | SEQ ID NO: 17 |
| hsa-miR-6075 | MIMAT0023700 | 0.794 | 2.77E−47 | SEQ ID NO: 18 |
| hsa-miR-5001-5p | MIMAT0021021 | 0.796 | 3.16E−46 | SEQ ID NO: 19 |
| hsa-miR-6789-5p | MIMAT0027478 | 0.683 | 6.98E−46 | SEQ ID NO: 20 |
| hsa-miR-4513 | MIMAT0019050 | 1.063 | 1.19E−45 | SEQ ID NO: 21 |
| hsa-miR-3192-5p | MIMAT0015076 | 4.111 | 1.76E−45 | SEQ ID NO: 22 |
| hsa-miR-8060 | MIMAT0030987 | 3.502 | 1.77E−45 | SEQ ID NO: 23 |
| hsa-miR-668-5p | MIMAT0026636 | 2.748 | 2.02E−45 | SEQ ID NO: 24 |
| hsa-miR-1268a | MIMAT0005922 | −0.610 | 2.40E−45 | SEQ ID NO: 25 |
| hsa-miR-1273g-3p | MIMAT0022742 | 1.448 | 2.67E−45 | SEQ ID NO: 26 |
| hsa-miR-4706 | MIMAT0019806 | 1.063 | 5.43E−45 | SEQ ID NO: 27 |
| hsa-miR-124-3p | MIMAT0000422 | 3.734 | 5.43E−45 | SEQ ID NO: 28 |
| hsa-miR-1260b | MIMAT0015041 | 1.278 | 9.38E−45 | SEQ ID NO: 29 |
| hsa-miR-4740-5p | MIMAT0019869 | 3.165 | 9.50E−45 | SEQ ID NO: 30 |
| hsa-miR-320b | MIMAT0005792 | 2.317 | 1.08E−44 | SEQ ID NO: 31 |
| hsa-miR-7977 | MIMAT0031180 | 1.267 | 4.78E−43 | SEQ ID NO: 32 |
| hsa-miR-29b-3p | MIMAT0000100 | 4.104 | 1.07E−42 | SEQ ID NO: 33 |
| hsa-miR-4708-3p | MIMAT0019810 | 2.780 | 2.73E−42 | SEQ ID NO: 34 |
| hsa-miR-4525 | MIMAT0019064 | 2.389 | 3.12E−42 | SEQ ID NO: 35 |
| hsa-miR-92b-3p | MIMAT0003218 | 2.494 | 3.43E−42 | SEQ ID NO: 36 |
| hsa-miR-4257 | MIMAT0016878 | 1.007 | 4.69E−42 | SEQ ID NO: 37 |
| hsa-miR-4727-3p | MIMAT0019848 | 2.681 | 7.55E−42 | SEQ ID NO: 38 |
| hsa-miR-92a-3p | MIMAT0000092 | 2.012 | 9.49E−42 | SEQ ID NO: 39 |
| hsa-miR-663a | MIMAT0003326 | 1.077 | 1.02E−41 | SEQ ID NO: 40 |
| hsa-miR-6787-5p | MIMAT0027474 | 1.234 | 5.33E−41 | SEQ ID NO: 41 |
| hsa-miR-3131 | MIMAT0014996 | 1.186 | 7.21E−41 | SEQ ID NO: 42 |
| hsa-miR-6802-5p | MIMAT0027504 | 0.851 | 2.03E−40 | SEQ ID NO: 43 |
| hsa-miR-654-5p | MIMAT0003330 | 2.538 | 3.90E−40 | SEQ ID NO: 44 |
| hsa-miR-6511b-5p | MIMAT0025847 | 1.931 | 1.70E−39 | SEQ ID NO: 45 |
| hsa-miR-29b-1-5p | MIMAT0004514 | 4.291 | 1.38E−38 | SEQ ID NO: 46 |
| hsa-miR-4417 | MIMAT0018929 | 0.424 | 1.66E−38 | SEQ ID NO: 47 |
| hsa-miR-4736 | MIMAT0019862 | 1.509 | 2.07E−38 | SEQ ID NO: 48 |
| hsa-miR-6840-3p | MIMAT0027583 | 0.913 | 3.82E−38 | SEQ ID NO: 49 |
| hsa-miR-4710 | MIMAT0019815 | 2.579 | 4.97E−38 | SEQ ID NO: 50 |
| hsa-miR-4635 | MIMAT0019692 | 2.883 | 6.15E−38 | SEQ ID NO: 51 |
| hsa-miR-296-3p | MIMAT0004679 | 1.513 | 1.29E−37 | SEQ ID NO: 52 |
| hsa-miR-1199-5p | MIMAT0031119 | 1.674 | 1.86E−37 | SEQ ID NO: 53 |
| hsa-miR-7975 | MIMAT0031178 | 1.390 | 2.19E−37 | SEQ ID NO: 54 |
| hsa-miR-4480 | MIMAT0019014 | 3.982 | 4.89E−37 | SEQ ID NO: 55 |
| hsa-miR-3648 | MIMAT0018068 | 0.970 | 5.72E−37 | SEQ ID NO: 56 |
| hsa-miR-371a-5p | MIMAT0004687 | 0.870 | 6.10E−37 | SEQ ID NO: 57 |
| hsa-miR-4771 | MIMAT0019925 | 3.676 | 8.98E−37 | SEQ ID NO: 58 |
| hsa-miR-6717-5p | MIMAT0025846 | 1.864 | 1.57E−36 | SEQ ID NO: 59 |
| hsa-miR-1254 | MIMAT0005905 | 1.180 | 1.74E−36 | SEQ ID NO: 60 |
| hsa-miR-1246 | MIMAT0005898 | 4.358 | 2.88E−36 | SEQ ID NO: 61 |
| hsa-miR-23b-3p | MIMAT0000418 | 3.275 | 4.55E−36 | SEQ ID NO: 62 |
| hsa-miR-320a | MIMAT0000510 | 1.560 | 6.90E−36 | SEQ ID NO: 63 |
| hsa-miR-4687-5p | MIMAT0019774 | 1.025 | 1.02E−35 | SEQ ID NO: 64 |
| hsa-miR-191-5p | MIMAT0000440 | 3.613 | 2.26E−35 | SEQ ID NO: 65 |
| hsa-miR-320c | MIMAT0005793 | 2.483 | 2.27E−35 | SEQ ID NO: 66 |
| hsa-miR-6131 | MIMAT0024615 | 3.119 | 4.64E−35 | SEQ ID NO: 67 |
| hsa-miR-4515 | MIMAT0019052 | 2.382 | 4.69E−35 | SEQ ID NO: 68 |
| hsa-miR-342-5p | MIMAT0004694 | 1.848 | 4.72E−35 | SEQ ID NO: 69 |
| hsa-miR-4718 | MIMAT0019831 | 3.469 | 4.73E−35 | SEQ ID NO: 70 |
| hsa-miR-23a-3p | MIMAT0000078 | 3.010 | 5.66E−35 | SEQ ID NO: 71 |
| hsa-miR-4455 | MIMAT0018977 | 2.615 | 6.45E−35 | SEQ ID NO: 72 |
| hsa-miR-211-3p | MIMAT0022694 | 1.267 | 1.43E−34 | SEQ ID NO: 73 |
| hsa-miR-3122 | MIMAT0014984 | 1.673 | 2.63E−34 | SEQ ID NO: 74 |
| hsa-miR-103a-3p | MIMAT0000101 | 3.839 | 4.23E−34 | SEQ ID NO: 75 |
| hsa-miR-4429 | MIMAT0018944 | 1.427 | 1.36E−33 | SEQ ID NO: 76 |
| hsa-miR-920 | MIMAT0004970 | 2.319 | 1.79E−33 | SEQ ID NO: 77 |
| hsa-miR-3194-3p | MIMAT0019218 | 3.177 | 1.93E−33 | SEQ ID NO: 78 |
| hsa-miR-4754 | MIMAT0019894 | 3.293 | 2.21E−33 | SEQ ID NO: 79 |
| hsa-miR-1238-5p | MIMAT0022947 | 1.377 | 2.33E−33 | SEQ ID NO: 80 |
| hsa-miR-3191-3p | MIMAT0015075 | 1.600 | 2.38E−33 | SEQ ID NO: 81 |
| hsa-miR-4755-3p | MIMAT0019896 | 3.798 | 3.45E−33 | SEQ ID NO: 82 |
| hsa-miR-3688-5p | MIMAT0019223 | 3.645 | 7.47E−33 | SEQ ID NO: 83 |
| hsa-miR-4529-5p | MIMAT0019236 | 3.453 | 1.07E−32 | SEQ ID NO: 84 |
| hsa-miR-6861-5p | MIMAT0027623 | 0.818 | 1.20E−32 | SEQ ID NO: 85 |
| hsa-miR-1469 | MIMAT0007347 | 0.758 | 2.45E−32 | SEQ ID NO: 86 |
| hsa-miR-619-5p | MIMAT0026622 | 1.750 | 2.88E−32 | SEQ ID NO: 87 |
| hsa-miR-4448 | MIMAT0018967 | 2.410 | 3.95E−32 | SEQ ID NO: 88 |
| hsa-miR-4658 | MIMAT0019725 | 2.788 | 4.02E−32 | SEQ ID NO: 89 |
| hsa-miR-22-3p | MIMAT0000077 | 2.815 | 5.70E−32 | SEQ ID NO: 90 |
| hsa-miR-4776-5p | MIMAT0019932 | 2.510 | 6.41E−32 | SEQ ID NO: 91 |
| hsa-miR-320e | MIMAT0015072 | 3.365 | 1.05E−31 | SEQ ID NO: 92 |
| hsa-miR-1225-3p | MIMAT0005573 | 0.741 | 1.99E−31 | SEQ ID NO: 93 |
| hsa-miR-6875-5p | MIMAT0027650 | −0.840 | 2.62E−31 | SEQ ID NO: 94 |
| hsa-miR-4534 | MIMAT0019073 | 1.324 | 3.60E−31 | SEQ ID NO: 95 |
| hsa-miR-4652-5p | MIMAT0019716 | 3.280 | 3.60E−31 | SEQ ID NO: 96 |
| hsa-miR-648 | MIMAT0003318 | 3.145 | 4.13E−31 | SEQ ID NO: 97 |
| hsa-miR-4259 | MIMAT0016880 | 2.262 | 4.13E−31 | SEQ ID NO: 98 |
| hsa-miR-107 | MIMAT0000104 | 3.642 | 6.38E−31 | SEQ ID NO: 99 |
| hsa-miR-650 | MIMAT0003320 | 2.399 | 7.82E−31 | SEQ ID NO: 100 |
| hsa-miR-3160-5p | MIMAT0019212 | 2.610 | 1.02E−30 | SEQ ID NO: 101 |
| hsa-miR-4697-5p | MIMAT0019791 | 0.755 | 1.71E−30 | SEQ ID NO: 102 |
| hsa-miR-2467-3p | MIMAT0019953 | 2.533 | 2.39E−30 | SEQ ID NO: 103 |
| hsa-miR-6501-3p | MIMAT0025459 | 2.859 | 6.98E−30 | SEQ ID NO: 104 |
| hsa-miR-6784-5p | MIMAT0027468 | −0.845 | 7.73E−30 | SEQ ID NO: 105 |
| hsa-miR-6760-5p | MIMAT0027420 | 2.468 | 8.54E−30 | SEQ ID NO: 106 |
| hsa-miR-151a-5p | MIMAT0004697 | 3.423 | 1.41E−29 | SEQ ID NO: 107 |
| hsa-miR-4700-5p | MIMAT0019796 | 3.186 | 1.52E−29 | SEQ ID NO: 108 |
| hsa-miR-6769a-5p | MIMAT0027438 | 1.060 | 2.47E−29 | SEQ ID NO: 109 |
| hsa-miR-3195 | MIMAT0015079 | 0.628 | 6.58E−29 | SEQ ID NO: 110 |
| hsa-miR-4489 | MIMAT0019023 | 2.385 | 1.39E−28 | SEQ ID NO: 111 |
| hsa-miR-1247-3p | MIMAT0022721 | 0.870 | 1.62E−28 | SEQ ID NO: 112 |
| hsa-miR-3622a-5p | MIMAT0018003 | 1.189 | 1.63E−28 | SEQ ID NO: 113 |
| hsa-miR-24-3p | MIMAT0000080 | 1.792 | 1.71E−28 | SEQ ID NO: 114 |
| hsa-miR-1260a | MIMAT0005911 | 0.973 | 1.78E−28 | SEQ ID NO: 115 |
| hsa-miR-614 | MIMAT0003282 | 1.620 | 2.20E−28 | SEQ ID NO: 116 |
| hsa-miR-6737-5p | MIMAT0027375 | 0.671 | 3.01E−28 | SEQ ID NO: 117 |
| hsa-miR-4294 | MIMAT0016849 | −0.649 | 3.57E−28 | SEQ ID NO: 118 |
| hsa-miR-3135b | MIMAT0018985 | 0.926 | 3.63E−28 | SEQ ID NO: 119 |
| hsa-miR-4633-5p | MIMAT0019689 | 3.035 | 6.27E−28 | SEQ ID NO: 120 |
| hsa-miR-320d | MIMAT0006764 | 2.747 | 7.22E−28 | SEQ ID NO: 121 |
| hsa-miR-4423-5p | MIMAT0019232 | 3.238 | 7.22E−28 | SEQ ID NO: 122 |
| hsa-miR-296-5p | MIMAT0000690 | 0.561 | 1.68E−27 | SEQ ID NO: 123 |
| hsa-miR-6836-3p | MIMAT0027575 | 0.841 | 2.72E−27 | SEQ ID NO: 124 |
| hsa-miR-598-5p | MIMAT0026620 | 2.843 | 3.03E−27 | SEQ ID NO: 125 |
| hsa-miR-373-5p | MIMAT0000725 | 0.795 | 3.56E−27 | SEQ ID NO: 126 |
| hsa-miR-4713-3p | MIMAT0019821 | 2.766 | 4.44E−27 | SEQ ID NO: 127 |
| hsa-miR-6076 | MIMAT0023701 | 0.742 | 1.03E−26 | SEQ ID NO: 128 |
| hsa-miR-3917 | MIMAT0018191 | 1.021 | 2.12E−26 | SEQ ID NO: 129 |
| hsa-miR-4296 | MIMAT0016845 | 2.839 | 2.36E−26 | SEQ ID NO: 130 |
| hsa-miR-1914-3p | MIMAT0007890 | 0.583 | 3.41E−26 | SEQ ID NO: 131 |
| hsa-miR-3162-5p | MIMAT0015036 | 0.618 | 7.15E−26 | SEQ ID NO: 132 |
| hsa-miR-4430 | MIMAT0018945 | 1.260 | 1.66E−25 | SEQ ID NO: 133 |
| hsa-miR-486-5p | MIMAT0002177 | 1.054 | 2.02E−25 | SEQ ID NO: 134 |
| hsa-miR-4447 | MIMAT0018966 | 1.105 | 2.02E−25 | SEQ ID NO: 135 |
| hsa-miR-6798-5p | MIMAT0027496 | −0.583 | 2.68E−25 | SEQ ID NO: 136 |
| hsa-miR-4747-3p | MIMAT0019883 | 1.124 | 3.29E−25 | SEQ ID NO: 137 |
| hsa-miR-151b | MIMAT0010214 | 3.223 | 6.37E−25 | SEQ ID NO: 138 |
| hsa-miR-3610 | MIMAT0017987 | 1.535 | 7.18E−25 | SEQ ID NO: 139 |
| hsa-miR-4666a-5p | MIMAT0019741 | 3.276 | 7.91E−25 | SEQ ID NO: 140 |
| hsa-miR-642b-3p | MIMAT0018444 | 1.035 | 8.34E−25 | SEQ ID NO: 141 |
| hsa-miR-519d-5p | MIMAT0026610 | 2.528 | 8.92E−25 | SEQ ID NO: 142 |
| hsa-miR-4439 | MIMAT0018957 | 2.760 | 1.13E−24 | SEQ ID NO: 143 |
| hsa-miR-4531 | MIMAT0019070 | 3.021 | 1.16E−24 | SEQ ID NO: 144 |
| hsa-miR-2110 | MIMAT0010133 | 1.064 | 1.17E−24 | SEQ ID NO: 145 |
| hsa-miR-140-3p | MIMAT0004597 | 3.010 | 1.73E−24 | SEQ ID NO: 146 |
| hsa-miR-4690-5p | MIMAT0019779 | 1.002 | 1.91E−24 | SEQ ID NO: 147 |
| hsa-miR-4663 | MIMAT0019735 | 2.723 | 2.33E−24 | SEQ ID NO: 148 |
| hsa-miR-518c-5p | MIMAT0002847 | 2.214 | 2.68E−24 | SEQ ID NO: 149 |
| hsa-miR-6781-5p | MIMAT0027462 | −0.418 | 2.89E−24 | SEQ ID NO: 150 |
| hsa-miR-1273g-5p | MIMAT0020602 | 2.614 | 3.15E−24 | SEQ ID NO: 151 |
| hsa-miR-4786-5p | MIMAT0019954 | 2.777 | 3.49E−24 | SEQ ID NO: 152 |
| hsa-miR-4538 | MIMAT0019081 | 2.624 | 4.11E−24 | SEQ ID NO: 153 |
| hsa-miR-4461 | MIMAT0018983 | 2.638 | 5.62E−24 | SEQ ID NO: 154 |
| hsa-miR-6073 | MIMAT0023698 | 3.254 | 7.97E−24 | SEQ ID NO: 155 |
| hsa-miR-6766-5p | MIMAT0027432 | 1.046 | 8.41E−24 | SEQ ID NO: 156 |
| hsa-miR-6762-5p | MIMAT0027424 | 0.739 | 9.08E−24 | SEQ ID NO: 157 |
| hsa-miR-561-5p | MIMAT0022706 | 2.143 | 9.95E−24 | SEQ ID NO: 158 |
| hsa-miR-4261 | MIMAT0016890 | 2.346 | 1.38E−23 | SEQ ID NO: 159 |
| hsa-miR-4532 | MIMAT0019071 | 1.508 | 1.42E−23 | SEQ ID NO: 160 |
| hsa-miR-1202 | MIMAT0005865 | 0.558 | 2.07E−23 | SEQ ID NO: 161 |
| hsa-miR-16-5p | MIMAT0000069 | 2.959 | 3.34E−23 | SEQ ID NO: 162 |
| hsa-miR-6515-5p | MIMAT0025486 | 1.398 | 3.78E−23 | SEQ ID NO: 163 |
| hsa-miR-6749-5p | MIMAT0027398 | 0.483 | 4.38E−23 | SEQ ID NO: 164 |
| hsa-miR-4436b-3p | MIMAT0019941 | 2.718 | 5.65E−23 | SEQ ID NO: 165 |
| hsa-miR-221-3p | MIMAT0000278 | 2.491 | 6.45E−23 | SEQ ID NO: 166 |
| hsa-miR-4707-5p | MIMAT0019807 | 0.422 | 7.06E−23 | SEQ ID NO: 167 |
| hsa-miR-4685-5p | MIMAT0019771 | 1.122 | 7.70E−23 | SEQ ID NO: 168 |
| hsa-miR-8059 | MIMAT0030986 | 1.269 | 9.16E−23 | SEQ ID NO: 169 |
| hsa-miR-664b-5p | MIMAT0022271 | 2.235 | 9.17E−23 | SEQ ID NO: 170 |
| hsa-miR-6765-3p | MIMAT0027431 | 0.923 | 1.85E−22 | SEQ ID NO: 171 |
| hsa-miR-6812-5p | MIMAT0027524 | 1.167 | 2.42E−22 | SEQ ID NO: 172 |
| hsa-miR-6826-5p | MIMAT0027552 | 0.871 | 2.70E−22 | SEQ ID NO: 173 |
| hsa-miR-6720-3p | MIMAT0025851 | 2.245 | 3.23E−22 | SEQ ID NO: 174 |
| hsa-miR-4475 | MIMAT0019002 | 2.786 | 3.59E−22 | SEQ ID NO: 175 |
| hsa-miR-551b-5p | MIMAT0004794 | 1.937 | 3.86E−22 | SEQ ID NO: 176 |
| hsa-miR-4419b | MIMAT0019034 | 1.162 | 4.25E−22 | SEQ ID NO: 177 |
| hsa-miR-3940-5p | MIMAT0019229 | −0.480 | 4.28E−22 | SEQ ID NO: 178 |
| hsa-miR-122-5p | MIMAT0000421 | 2.710 | 5.61E−22 | SEQ ID NO: 179 |
| hsa-miR-3156-5p | MIMAT0015030 | 1.835 | 6.67E−22 | SEQ ID NO: 180 |
| hsa-miR-6885-5p | MIMAT0027670 | 0.723 | 9.36E−22 | SEQ ID NO: 181 |
| hsa-miR-3150a-3p | MIMAT0015023 | 1.608 | 9.79E−22 | SEQ ID NO: 182 |
| hsa-miR-4514 | MIMAT0019051 | 2.815 | 1.10E−21 | SEQ ID NO: 183 |
| hsa-miR-3652 | MIMAT0018072 | 0.894 | 1.14E−21 | SEQ ID NO: 184 |
| hsa-miR-3714 | MIMAT0018165 | 2.329 | 1.44E−21 | SEQ ID NO: 185 |
| hsa-miR-130a-3p | MIMAT0000425 | 2.728 | 1.92E−21 | SEQ ID NO: 186 |
| hsa-miR-4764-5p | MIMAT0019914 | 2.173 | 2.47E−21 | SEQ ID NO: 187 |
| hsa-miR-4783-3p | MIMAT0019947 | 1.443 | 4.18E−21 | SEQ ID NO: 188 |
| hsa-miR-4724-3p | MIMAT0019842 | 2.791 | 4.43E−21 | SEQ ID NO: 189 |
| hsa-miR-4529-3p | MIMAT0019068 | 2.334 | 4.43E−21 | SEQ ID NO: 190 |
| hsa-miR-937-5p | MIMAT0022938 | 0.587 | 5.05E−21 | SEQ ID NO: 191 |
| hsa-miR-4500 | MIMAT0019036 | 2.450 | 5.79E−21 | SEQ ID NO: 192 |
| hsa-miR-4444 | MIMAT0018962 | 2.342 | 6.32E−21 | SEQ ID NO: 193 |
| hsa-let-7a-5p | MIMAT0000062 | 2.776 | 6.53E−21 | SEQ ID NO: 194 |
| hsa-miR-519c-5p, | MIMAT0002831, | 2.491 | 6.55E−21 | SEQ ID NO: 195 |
| hsa-miR-523-5p, | MIMAT0005449, | | | |
| hsa-miR-518e-5p, | MIMAT0005450, | | | |
| hsa-miR-522-5p, | MIMAT0005451, | | | |
| hsa-miR-519a-5p, | MIMAT0005452, | | | |
| hsa-miR-519b-5p | MIMAT0005454 | | | |
| hsa-miR-4770 | MIMAT0019924 | 2.608 | 6.62E−21 | SEQ ID NO: 196 |
| hsa-miR-4634 | MIMAT0019691 | 0.558 | 6.97E−21 | SEQ ID NO: 197 |
| hsa-miR-6859-5p | MIMAT0027618 | 2.406 | 7.95E−21 | SEQ ID NO: 198 |
| hsa-miR-3170 | MIMAT0015045 | 2.385 | 8.60E−21 | SEQ ID NO: 199 |
| hsa-miR-6718-5p | MIMAT0025849 | 2.355 | 1.00E−20 | SEQ ID NO: 200 |
| hsa-miR-6838-3p | MIMAT0027579 | 2.603 | 1.06E−20 | SEQ ID NO: 201 |
| hsa-miR-3934-5p | MIMAT0018349 | 1.971 | 1.08E−20 | SEQ ID NO: 202 |
| hsa-miR-1914-5p | MIMAT0007889 | 1.323 | 1.18E−20 | SEQ ID NO: 203 |
| hsa-miR-6752-5p | MIMAT0027404 | −0.311 | 1.33E−20 | SEQ ID NO: 204 |
| hsa-miR-524-5p | MIMAT0002849 | 2.616 | 1.94E−20 | SEQ ID NO: 205 |
| hsa-miR-6722-5p | MIMAT0025853 | 1.203 | 2.12E−20 | SEQ ID NO: 206 |
| hsa-miR-875-5p | MIMAT0004922 | 1.857 | 2.40E−20 | SEQ ID NO: 207 |
| hsa-miR-6872-5p | MIMAT0027644 | 2.413 | 2.63E−20 | SEQ ID NO: 208 |
| hsa-miR-4434 | MIMAT0018950 | 2.914 | 3.39E−20 | SEQ ID NO: 209 |
| hsa-miR-3977 | MIMAT0019362 | 2.408 | 3.98E−20 | SEQ ID NO: 210 |
| hsa-miR-575 | MIMAT0003240 | −1.525 | 4.37E−20 | SEQ ID NO: 211 |
| hsa-miR-5586-3p | MIMAT0022288 | 2.641 | 4.39E−20 | SEQ ID NO: 212 |
| hsa-miR-6724-5p | MIMAT0025856 | 0.326 | 4.46E−20 | SEQ ID NO: 213 |
| hsa-miR-4665-5p | MIMAT0019739 | 0.494 | 4.65E−20 | SEQ ID NO: 214 |
| hsa-miR-3661 | MIMAT0018082 | 2.269 | 4.87E−20 | SEQ ID NO: 215 |
| hsa-miR-658 | MIMAT0003336 | 0.910 | 4.98E−20 | SEQ ID NO: 216 |
| hsa-miR-4732-5p | MIMAT0019855 | 1.821 | 6.01E−20 | SEQ ID NO: 217 |
| hsa-miR-3136-3p | MIMAT0019203 | 2.141 | 6.52E−20 | SEQ ID NO: 218 |
| hsa-miR-3927-3p | MIMAT0018202 | 2.118 | 6.75E−20 | SEQ ID NO: 219 |
| hsa-miR-4322 | MIMAT0016873 | 0.875 | 8.57E−20 | SEQ ID NO: 220 |
| hsa-miR-4436b-5p | MIMAT0019940 | 0.977 | 1.01E−19 | SEQ ID NO: 221 |
| hsa-miR-5681a | MIMAT0022469 | 2.224 | 1.04E−19 | SEQ ID NO: 222 |
| hsa-miR-4705 | MIMAT0019805 | 2.413 | 1.10E−19 | SEQ ID NO: 223 |
| hsa-miR-6794-5p | MIMAT0027488 | 0.488 | 1.15E−19 | SEQ ID NO: 224 |
| hsa-miR-4779 | MIMAT0019938 | 2.279 | 1.20E−19 | SEQ ID NO: 225 |
| hsa-miR-4795-3p | MIMAT0019969 | 2.776 | 1.32E−19 | SEQ ID NO: 226 |
| hsa-miR-1471 | MIMAT0007349 | 1.715 | 1.36E−19 | SEQ ID NO: 227 |
| hsa-miR-4633-3p | MIMAT0019690 | 2.888 | 1.43E−19 | SEQ ID NO: 228 |
| hsa-miR-15b-5p | MIMAT0000417 | 2.433 | 1.59E−19 | SEQ ID NO: 229 |
| hsa-miR-526a, | MIMAT0002845, | 2.258 | 1.64E−19 | SEQ ID NO: 230 |
| hsa-miR-520c-5p, | MIMAT0005455, | | | |
| hsa-miR-518d-5p | MIMAT0005456 | | | |
| hsa-miR-3936 | MIMAT0018351 | 2.508 | 1.64E−19 | SEQ ID NO: 231 |
| hsa-miR-210-5p | MIMAT0026475 | 1.017 | 2.11E−19 | SEQ ID NO: 232 |
| hsa-miR-3165 | MIMAT0015039 | 2.355 | 2.21E−19 | SEQ ID NO: 233 |
| hsa-miR-4757-5p | MIMAT0019901 | 2.753 | 2.30E−19 | SEQ ID NO: 234 |
| hsa-miR-4457 | MIMAT0018979 | 2.429 | 3.13E−19 | SEQ ID NO: 235 |
| hsa-miR-4789-5p | MIMAT0019959 | 2.715 | 3.31E−19 | SEQ ID NO: 236 |
| hsa-miR-6887-5p | MIMAT0027674 | 0.944 | 3.91E−19 | SEQ ID NO: 237 |
| hsa-miR-3924 | MIMAT0018199 | 1.994 | 3.95E−19 | SEQ ID NO: 238 |
| hsa-miR-5579-3p | MIMAT0022270 | 2.116 | 4.53E−19 | SEQ ID NO: 239 |
| hsa-miR-4464 | MIMAT0018988 | 2.163 | 4.54E−19 | SEQ ID NO: 240 |
| hsa-miR-4786-3p | MIMAT0019955 | 2.036 | 4.69E−19 | SEQ ID NO: 241 |
| hsa-miR-885-3p | MIMAT0004948 | 1.342 | 4.73E−19 | SEQ ID NO: 242 |
| hsa-miR-128-2-5p | MIMAT0031095 | 0.725 | 5.18E−19 | SEQ ID NO: 243 |
| hsa-miR-26a-5p | MIMAT0000082 | 2.512 | 5.40E−19 | SEQ ID NO: 244 |
| hsa-miR-4703-5p | MIMAT0019801 | 2.127 | 6.97E−19 | SEQ ID NO: 245 |
| hsa-miR-5591-5p | MIMAT0022301 | 2.160 | 7.33E−19 | SEQ ID NO: 246 |
| hsa-miR-4777-3p | MIMAT0019935 | 2.661 | 7.58E−19 | SEQ ID NO: 247 |
| hsa-miR-4729 | MIMAT0019851 | 2.483 | 8.33E−19 | SEQ ID NO: 248 |
| hsa-miR-345-3p | MIMAT0022698 | 0.857 | 9.21E−19 | SEQ ID NO: 249 |
| hsa-miR-6515-3p | MIMAT0025487 | 0.538 | 9.58E−19 | SEQ ID NO: 250 |
| hsa-miR-5195-3p | MIMAT0021127 | 0.736 | 1.12E−18 | SEQ ID NO: 251 |
| hsa-miR-4719 | MIMAT0019832 | 2.263 | 1.19E−18 | SEQ ID NO: 252 |
| hsa-miR-3151-5p | MIMAT0015024 | 1.208 | 1.21E−18 | SEQ ID NO: 253 |
| hsa-miR-99a-5p | MIMAT0000097 | 2.558 | 1.54E−18 | SEQ ID NO: 254 |
| hsa-miR-4687-3p | MIMAT0019775 | −0.273 | 1.61E−18 | SEQ ID NO: 255 |
| hsa-miR-3121-5p | MIMAT0019199 | 2.553 | 1.61E−18 | SEQ ID NO: 256 |
| hsa-miR-148b-5p | MIMAT0004699 | 2.362 | 1.63E−18 | SEQ ID NO: 257 |
| hsa-miR-3689a-5p, | MIMAT0018117, | 1.780 | 1.71E−18 | SEQ ID NO: 258 |
| hsa-miR-3689b-5p, | MIMAT0018180, | | | |
| hsa-miR-3689e | MIMAT0019009 | | | |
| hsa-miR-8052 | MIMAT0030979 | 1.029 | 1.72E−18 | SEQ ID NO: 259 |
| hsa-miR-1249-3p | MIMAT0005901 | 0.567 | 1.80E−18 | SEQ ID NO: 260 |
| hsa-miR-1228-3p | MIMAT0005583 | 0.428 | 1.83E−18 | SEQ ID NO: 261 |
| hsa-miR-4774-5p | MIMAT0019929 | 2.438 | 1.95E−18 | SEQ ID NO: 262 |
| hsa-miR-4678 | MIMAT0019762 | 2.008 | 2.00E−18 | SEQ ID NO: 263 |
| hsa-miR-302c-5p | MIMAT0000716 | 1.715 | 2.03E−18 | SEQ ID NO: 264 |
| hsa-miR-193a-5p | MIMAT0004614 | 1.700 | 2.07E−18 | SEQ ID NO: 265 |
| hsa-miR-4536-5p | MIMAT0019078 | 2.230 | 2.13E−18 | SEQ ID NO: 266 |
| hsa-miR-505-5p | MIMAT0004776 | 2.100 | 2.15E−18 | SEQ ID NO: 267 |
| hsa-miR-208b-5p | MIMAT0026722 | 2.038 | 2.17E−18 | SEQ ID NO: 268 |
| hsa-let-7b-5p | MIMAT0000063 | 2.479 | 2.17E−18 | SEQ ID NO: 269 |
| hsa-miR-7854-3p | MIMAT0030429 | 1.672 | 2.21E−18 | SEQ ID NO: 270 |
| hsa-miR-3908 | MIMAT0018182 | 1.902 | 2.43E−18 | SEQ ID NO: 271 |
| hsa-miR-8064 | MIMAT0030991 | 1.532 | 2.43E−18 | SEQ ID NO: 272 |
| hsa-miR-1287-5p | MIMAT0005878 | 2.285 | 2.46E−18 | SEQ ID NO: 273 |
| hsa-miR-649 | MIMAT0003319 | 2.305 | 2.50E−18 | SEQ ID NO: 274 |
| hsa-miR-145-5p | MIMAT0000437 | 2.396 | 2.72E−18 | SEQ ID NO: 275 |
| hsa-miR-6748-5p | MIMAT0027396 | 1.388 | 2.86E−18 | SEQ ID NO: 276 |
| hsa-miR-25-3p | MIMAT0000081 | 2.253 | 2.93E−18 | SEQ ID NO: 277 |
| hsa-miR-520f-5p | MIMAT0026609 | 2.154 | 2.99E−18 | SEQ ID NO: 278 |
| hsa-miR-3923 | MIMAT0018198 | 1.814 | 3.06E−18 | SEQ ID NO: 279 |
| hsa-miR-6819-5p | MIMAT0027538 | 0.396 | 3.32E−18 | SEQ ID NO: 280 |
| hsa-miR-219b-3p | MIMAT0019748 | 2.382 | 3.75E−18 | SEQ ID NO: 281 |
| hsa-miR-3158-5p | MIMAT0019211 | 1.152 | 4.10E−18 | SEQ ID NO: 282 |
| hsa-miR-6793-5p | MIMAT0027486 | 1.967 | 4.12E−18 | SEQ ID NO: 283 |
| hsa-miR-223-3p | MIMAT0000280 | 2.057 | 4.12E−18 | SEQ ID NO: 284 |
| hsa-miR-7109-5p | MIMAT0028115 | 0.442 | 4.28E−18 | SEQ ID NO: 285 |
| hsa-miR-548bb-3p | MIMAT0035704 | 2.172 | 4.98E−18 | SEQ ID NO: 286 |
| hsa-let-7c-5p | MIMAT0000064 | 2.686 | 5.06E−18 | SEQ ID NO: 287 |
| hsa-miR-605-3p | MIMAT0026621 | 2.208 | 5.09E−18 | SEQ ID NO: 288 |
| hsa-miR-5091 | MIMAT0021083 | 2.314 | 5.22E−18 | SEQ ID NO: 289 |
| hsa-miR-3117-5p | MIMAT0019197 | 1.877 | 5.22E−18 | SEQ ID NO: 290 |
| hsa-miR-4778-3p | MIMAT0019937 | 1.959 | 5.64E−18 | SEQ ID NO: 291 |
| hsa-miR-514a-3p | MIMAT0002883 | 2.460 | 5.69E−18 | SEQ ID NO: 292 |
| hsa-miR-3115 | MIMAT0014977 | 1.790 | 5.71E−18 | SEQ ID NO: 293 |
| hsa-miR-4795-5p | MIMAT0019968 | 1.955 | 5.84E−18 | SEQ ID NO: 294 |
| hsa-miR-627-3p | MIMAT0026623 | 2.249 | 6.38E−18 | SEQ ID NO: 295 |
| hsa-miR-4642 | MIMAT0019702 | 2.311 | 6.41E−18 | SEQ ID NO: 296 |
| hsa-miR-4753-5p | MIMAT0019890 | 2.369 | 7.32E−18 | SEQ ID NO: 297 |
| hsa-miR-6822-5p | MIMAT0027544 | 1.449 | 7.59E−18 | SEQ ID NO: 298 |
| hsa-miR-498 | MIMAT0002824 | 0.687 | 8.12E−18 | SEQ ID NO: 299 |
| hsa-miR-514b-5p | MIMAT0015087 | 1.708 | 8.22E−18 | SEQ ID NO: 300 |
| hsa-miR-522-3p | MIMAT0002868 | 2.443 | 9.19E−18 | SEQ ID NO: 301 |
| hsa-miR-6512-5p | MIMAT0025480 | 1.899 | 1.26E−17 | SEQ ID NO: 302 |
| hsa-miR-4650-3p | MIMAT0019714 | 2.452 | 1.27E−17 | SEQ ID NO: 303 |
| hsa-miR-6757-5p | MIMAT0027414 | 0.829 | 1.42E−17 | SEQ ID NO: 304 |
| hsa-miR-520d-5p | MIMAT0002855 | 1.924 | 1.78E−17 | SEQ ID NO: 305 |
| hsa-miR-365a-5p | MIMAT0009199 | 0.662 | 1.89E−17 | SEQ ID NO: 306 |
| hsa-miR-5197-3p | MIMAT0021131 | 1.986 | 2.04E−17 | SEQ ID NO: 307 |
| hsa-miR-4482-5p | MIMAT0019016 | 1.800 | 2.31E−17 | SEQ ID NO: 308 |
| hsa-miR-655-5p | MIMAT0026626 | 1.768 | 2.31E−17 | SEQ ID NO: 309 |
| hsa-miR-6511a-5p | MIMAT0025478 | 0.704 | 2.46E−17 | SEQ ID NO: 310 |
| hsa-miR-5706 | MIMAT0022500 | 2.382 | 2.58E−17 | SEQ ID NO: 311 |
| hsa-miR-548ah-5p | MIMAT0018972 | 1.681 | 2.69E−17 | SEQ ID NO: 312 |
| hsa-miR-5189-5p | MIMAT0021120 | 1.376 | 2.70E−17 | SEQ ID NO: 313 |
| hsa-miR-4453 | MIMAT0018975 | 1.971 | 3.41E−17 | SEQ ID NO: 314 |
| hsa-miR-3941 | MIMAT0018357 | 1.723 | 3.66E−17 | SEQ ID NO: 315 |
| hsa-miR-4756-5p | MIMAT0019899 | 1.247 | 3.72E−17 | SEQ ID NO: 316 |
| hsa-miR-590-5p | MIMAT0003258 | 2.073 | 3.90E−17 | SEQ ID NO: 317 |
| hsa-miR-548ad-3p | MIMAT0018946 | 1.846 | 4.12E−17 | SEQ ID NO: 318 |
| hsa-miR-3179 | MIMAT0015056 | 2.019 | 4.19E−17 | SEQ ID NO: 319 |
| hsa-miR-5689 | MIMAT0022481 | 2.056 | 4.32E−17 | SEQ ID NO: 320 |
| hsa-miR-376c-5p | MIMAT0022861 | 1.827 | 4.45E−17 | SEQ ID NO: 321 |
| hsa-miR-4490 | MIMAT0019025 | 2.488 | 4.46E−17 | SEQ ID NO: 322 |
| hsa-miR-526b-5p | MIMAT0002835 | 2.192 | 6.08E−17 | SEQ ID NO: 323 |
| hsa-miR-4693-3p | MIMAT0019785 | 2.164 | 6.24E−17 | SEQ ID NO: 324 |
| hsa-miR-1307-3p | MIMAT0005951 | 1.399 | 7.35E−17 | SEQ ID NO: 325 |
| hsa-miR-3686 | MIMAT0018114 | 1.682 | 7.55E−17 | SEQ ID NO: 326 |
| hsa-miR-4675 | MIMAT0019757 | 0.782 | 8.65E−17 | SEQ ID NO: 327 |
| hsa-miR-20b-5p | MIMAT0001413 | 2.412 | 9.28E−17 | SEQ ID NO: 328 |
| hsa-miR-3660 | MIMAT0018081 | 1.939 | 9.59E−17 | SEQ ID NO: 329 |
| hsa-miR-3150a-5p | MIMAT0019206 | 2.189 | 9.59E−17 | SEQ ID NO: 330 |
| hsa-miR-6507-5p | MIMAT0025470 | 2.265 | 1.07E−16 | SEQ ID NO: 331 |
| hsa-miR-3663-3p | MIMAT0018085 | 0.516 | 1.10E−16 | SEQ ID NO: 332 |
| hsa-miR-555 | MIMAT0003219 | 2.229 | 1.17E−16 | SEQ ID NO: 333 |
| hsa-miR-6786-5p | MIMAT0027472 | −0.313 | 1.23E−16 | SEQ ID NO: 334 |
| hsa-miR-379-5p | MIMAT0000733 | 2.400 | 1.27E−16 | SEQ ID NO: 335 |
| hsa-miR-548av-5p | MIMAT0022303 | 1.902 | 1.27E−16 | SEQ ID NO: 336 |
| hsa-miR-1258 | MIMAT0005909 | 2.054 | 1.35E−16 | SEQ ID NO: 337 |
| hsa-miR-29b-2-5p | MIMAT0004515 | 2.490 | 1.51E−16 | SEQ ID NO: 338 |
| hsa-miR-202-3p | MIMAT0002811 | 2.300 | 1.53E−16 | SEQ ID NO: 339 |
| hsa-miR-3155a | MIMAT0015029 | 1.941 | 1.53E−16 | SEQ ID NO: 340 |
| hsa-miR-27b-3p | MIMAT0000419 | 2.340 | 1.59E−16 | SEQ ID NO: 341 |
| hsa-miR-6849-5p | MIMAT0027598 | 1.811 | 1.63E−16 | SEQ ID NO: 342 |
| hsa-miR-15a-5p | MIMAT0000068 | 2.364 | 1.76E−16 | SEQ ID NO: 343 |
| hsa-miR-5197-5p | MIMAT0021130 | 2.204 | 1.85E−16 | SEQ ID NO: 344 |
| hsa-miR-5704 | MIMAT0022498 | 2.376 | 1.88E−16 | SEQ ID NO: 345 |
| hsa-miR-3133 | MIMAT0014998 | 2.010 | 2.14E−16 | SEQ ID NO: 346 |
| hsa-miR-4696 | MIMAT0019790 | 1.897 | 2.26E−16 | SEQ ID NO: 347 |
| hsa-miR-3124-5p | MIMAT0014986 | 1.842 | 2.31E−16 | SEQ ID NO: 348 |
| hsa-miR-2114-3p | MIMAT0011157 | 2.245 | 2.36E−16 | SEQ ID NO: 349 |
| hsa-miR-6830-5p | MIMAT0027560 | 1.429 | 2.57E−16 | SEQ ID NO: 350 |
| hsa-miR-4530 | MIMAT0019069 | −0.468 | 2.64E−16 | SEQ ID NO: 351 |
| hsa-miR-4477a | MIMAT0019004 | 2.039 | 2.67E−16 | SEQ ID NO: 352 |
| hsa-miR-126-3p | MIMAT0000445 | 2.285 | 2.84E−16 | SEQ ID NO: 353 |
| hsa-miR-1537-5p | MIMAT0026765 | 1.920 | 2.85E−16 | SEQ ID NO: 354 |
| hsa-miR-4451 | MIMAT0018973 | 2.157 | 2.89E−16 | SEQ ID NO: 355 |
| hsa-miR-217 | MIMAT0000274 | 2.178 | 2.91E−16 | SEQ ID NO: 356 |
| hsa-miR-510-5p | MIMAT0002882 | 2.354 | 3.09E−16 | SEQ ID NO: 357 |
| hsa-miR-548y | MIMAT0018354 | 1.725 | 3.10E−16 | SEQ ID NO: 358 |
| hsa-miR-3655 | MIMAT0018075 | 2.131 | 3.11E−16 | SEQ ID NO: 359 |
| hsa-miR-4679 | MIMAT0019763 | 2.023 | 3.15E−16 | SEQ ID NO: 360 |
| hsa-miR-19b-3p | MIMAT0000074 | 2.172 | 3.31E−16 | SEQ ID NO: 361 |
| hsa-miR-4460 | MIMAT0018982 | 2.683 | 3.39E−16 | SEQ ID NO: 362 |
| hsa-miR-190b | MIMAT0004929 | 1.856 | 3.47E−16 | SEQ ID NO: 363 |
| hsa-miR-4739 | MIMAT0019868 | −0.308 | 3.99E−16 | SEQ ID NO: 364 |
| hsa-miR-601 | MIMAT0003269 | 2.245 | 4.36E−16 | SEQ ID NO: 365 |
| hsa-miR-3670 | MIMAT0018093 | 2.172 | 4.36E−16 | SEQ ID NO: 366 |
| hsa-miR-2116-5p | MIMAT0011160 | 1.775 | 4.54E−16 | SEQ ID NO: 367 |
| hsa-miR-298 | MIMAT0004901 | 2.110 | 5.02E−16 | SEQ ID NO: 368 |
| hsa-miR-3153 | MIMAT0015026 | 1.516 | 5.15E−16 | SEQ ID NO: 369 |
| hsa-miR-4747-5p | MIMAT0019882 | 1.575 | 5.44E−16 | SEQ ID NO: 370 |
| hsa-miR-509-3p | MIMAT0002881 | 2.258 | 5.44E−16 | SEQ ID NO: 371 |
| hsa-miR-4317 | MIMAT0016872 | 2.440 | 6.15E−16 | SEQ ID NO: 372 |
| hsa-miR-1468-3p | MIMAT0026638 | 2.049 | 6.17E−16 | SEQ ID NO: 373 |
| hsa-miR-8062 | MIMAT0030989 | 1.938 | 6.19E−16 | SEQ ID NO: 374 |
| hsa-miR-4804-5p | MIMAT0019984 | 1.967 | 6.27E−16 | SEQ ID NO: 375 |
| hsa-miR-4303 | MIMAT0016856 | 1.977 | 6.49E−16 | SEQ ID NO: 376 |
| hsa-miR-5588-5p | MIMAT0022295 | 1.949 | 6.57E−16 | SEQ ID NO: 377 |
| hsa-miR-4783-5p | MIMAT0019946 | 1.476 | 7.23E−16 | SEQ ID NO: 378 |
| hsa-miR-5586-5p | MIMAT0022287 | 2.054 | 7.75E−16 | SEQ ID NO: 379 |
| hsa-miR-3613-5p | MIMAT0017990 | 1.525 | 8.05E−16 | SEQ ID NO: 380 |
| hsa-miR-411-5p | MIMAT0003329 | 2.248 | 8.30E−16 | SEQ ID NO: 381 |
| hsa-miR-4535 | MIMAT0019075 | 1.267 | 8.45E−16 | SEQ ID NO: 382 |
| hsa-miR-665 | MIMAT0004952 | 0.522 | 8.87E−16 | SEQ ID NO: 383 |
| hsa-miR-3912-3p | MIMAT0018186 | 1.683 | 9.10E−16 | SEQ ID NO: 384 |
| hsa-miR-513c-3p | MIMAT0022728 | 1.703 | 9.26E−16 | SEQ ID NO: 385 |
| hsa-miR-4774-3p | MIMAT0019930 | 1.733 | 9.36E−16 | SEQ ID NO: 386 |
| hsa-miR-1181 | MIMAT0005826 | 0.887 | 9.71E−16 | SEQ ID NO: 387 |
| hsa-miR-620 | MIMAT0003289 | 1.836 | 9.78E−16 | SEQ ID NO: 388 |
| hsa-miR-4743-5p | MIMAT0019874 | 1.340 | 9.81E−16 | SEQ ID NO: 389 |
| hsa-miR-6769b-5p | MIMAT0027620 | 0.650 | 1.03E−15 | SEQ ID NO: 390 |
| hsa-miR-604 | MIMAT0003272 | 2.191 | 1.03E−15 | SEQ ID NO: 391 |
| hsa-miR-4738-3p | MIMAT0019867 | 1.174 | 1.03E−15 | SEQ ID NO: 392 |
| hsa-miR-671-5p | MIMAT0003880 | 0.624 | 1.07E−15 | SEQ ID NO: 393 |
| hsa-miR-6729-3p | MIMAT0027360 | 0.585 | 1.15E−15 | SEQ ID NO: 394 |
| hsa-miR-301a-5p | MIMAT0022696 | 2.081 | 1.21E−15 | SEQ ID NO: 395 |
| hsa-miR-17-5p | MIMAT0000070 | 2.271 | 1.22E−15 | SEQ ID NO: 396 |
| hsa-miR-541-3p | MIMAT0004920 | 2.222 | 1.26E−15 | SEQ ID NO: 397 |
| hsa-miR-4777-5p | MIMAT0019934 | 1.755 | 1.26E−15 | SEQ ID NO: 398 |
| hsa-miR-4999-3p | MIMAT0021018 | 1.684 | 1.30E−15 | SEQ ID NO: 399 |
| hsa-miR-4477b | MIMAT0019005 | 1.861 | 1.30E−15 | SEQ ID NO: 400 |
| hsa-miR-4999-5p | MIMAT0021017 | 1.645 | 1.35E−15 | SEQ ID NO: 401 |
| hsa-miR-7641 | MIMAT0029782 | 1.211 | 1.37E−15 | SEQ ID NO: 402 |
| hsa-miR-5692a | MIMAT0022484 | 2.131 | 1.44E−15 | SEQ ID NO: 403 |
| hsa-miR-7110-5p | MIMAT0028117 | −0.856 | 1.70E−15 | SEQ ID NO: 404 |
| hsa-miR-4533 | MIMAT0019072 | 1.237 | 1.76E−15 | SEQ ID NO: 405 |
| hsa-miR-3683 | MIMAT0018111 | 1.849 | 1.77E−15 | SEQ ID NO: 406 |
| hsa-miR-30c-2-3p | MIMAT0004550 | 2.198 | 1.81E−15 | SEQ ID NO: 407 |
| hsa-miR-4520-3p | MIMAT0019057 | 1.700 | 1.86E−15 | SEQ ID NO: 408 |
| hsa-miR-4473 | MIMAT0019000 | 2.060 | 1.90E−15 | SEQ ID NO: 409 |
| hsa-miR-4768-3p | MIMAT0019921 | 1.842 | 1.94E−15 | SEQ ID NO: 410 |
| hsa-miR-1910-3p | MIMAT0026917 | 1.979 | 1.97E−15 | SEQ ID NO: 411 |
| hsa-miR-6874-5p | MIMAT0027648 | 1.940 | 1.98E−15 | SEQ ID NO: 412 |
| hsa-miR-4536-3p | MIMAT0020959 | 1.857 | 2.04E−15 | SEQ ID NO: 413 |
| hsa-miR-4715-5p | MIMAT0019824 | 1.849 | 2.17E−15 | SEQ ID NO: 414 |
| hsa-miR-1276 | MIMAT0005930 | 2.010 | 2.32E−15 | SEQ ID NO: 415 |
| hsa-miR-6834-5p | MIMAT0027568 | 1.767 | 2.40E−15 | SEQ ID NO: 416 |
| hsa-miR-511-3p | MIMAT0026606 | 2.147 | 2.48E−15 | SEQ ID NO: 417 |
| hsa-miR-3657 | MIMAT0018077 | 2.088 | 2.53E−15 | SEQ ID NO: 418 |
| hsa-miR-424-3p | MIMAT0004749 | 2.169 | 2.57E−15 | SEQ ID NO: 419 |
| hsa-miR-4299 | MIMAT0016851 | 2.071 | 2.66E−15 | SEQ ID NO: 420 |
| hsa-miR-888-3p | MIMAT0004917 | 2.099 | 2.71E−15 | SEQ ID NO: 421 |
| hsa-miR-210-3p | MIMAT0000267 | 1.597 | 2.75E−15 | SEQ ID NO: 422 |
| hsa-miR-4758-5p | MIMAT0019903 | 0.507 | 3.02E−15 | SEQ ID NO: 423 |
| hsa-miR-5682 | MIMAT0022470 | 1.578 | 3.08E−15 | SEQ ID NO: 424 |
| hsa-miR-4492 | MIMAT0019027 | 0.371 | 3.12E−15 | SEQ ID NO: 425 |
| hsa-miR-1226-5p | MIMAT0005576 | 1.857 | 3.30E−15 | SEQ ID NO: 426 |
| hsa-miR-6513-5p | MIMAT0025482 | 1.807 | 3.32E−15 | SEQ ID NO: 427 |
| hsa-miR-6882-5p | MIMAT0027664 | 1.871 | 3.35E−15 | SEQ ID NO: 428 |
| hsa-miR-3672 | MIMAT0018095 | 1.867 | 3.37E−15 | SEQ ID NO: 429 |
| hsa-miR-874-3p | MIMAT0004911 | 0.826 | 3.45E−15 | SEQ ID NO: 430 |
| hsa-miR-1285-3p | MIMAT0005876 | 1.795 | 3.48E−15 | SEQ ID NO: 431 |
| hsa-miR-5096 | MIMAT0020603 | 1.754 | 3.51E−15 | SEQ ID NO: 432 |
| hsa-miR-7852-3p | MIMAT0030427 | 1.881 | 3.53E−15 | SEQ ID NO: 433 |
| hsa-miR-617 | MIMAT0003286 | 2.092 | 3.55E−15 | SEQ ID NO: 434 |
| hsa-miR-924 | MIMAT0004974 | 1.706 | 3.58E−15 | SEQ ID NO: 435 |
| hsa-miR-6806-5p | MIMAT0027512 | 0.800 | 3.85E−15 | SEQ ID NO: 436 |
| hsa-miR-940 | MIMAT0004983 | 0.548 | 3.95E−15 | SEQ ID NO: 437 |
| hsa-miR-4301 | MIMAT0016850 | 2.117 | 3.97E−15 | SEQ ID NO: 438 |
| hsa-miR-4267 | MIMAT0016893 | 2.085 | 3.97E−15 | SEQ ID NO: 439 |
| hsa-miR-4694-5p | MIMAT0019786 | 2.230 | 4.08E−15 | SEQ ID NO: 440 |
| hsa-miR-4304 | MIMAT0016854 | 1.602 | 4.22E−15 | SEQ ID NO: 441 |
| hsa-miR-4711-3p | MIMAT0019817 | 2.315 | 4.57E−15 | SEQ ID NO: 442 |
| hsa-miR-2467-5p | MIMAT0019952 | 1.792 | 4.72E−15 | SEQ ID NO: 443 |
| hsa-let-7d-5p | MIMAT0000065 | 2.369 | 4.76E−15 | SEQ ID NO: 444 |
| hsa-miR-8076 | MIMAT0031003 | 1.807 | 5.24E−15 | SEQ ID NO: 445 |
| hsa-miR-5705 | MIMAT0022499 | 1.413 | 5.70E−15 | SEQ ID NO: 446 |
| hsa-miR-4660 | MIMAT0019728 | 1.834 | 5.70E−15 | SEQ ID NO: 447 |
| hsa-miR-1245b-3p | MIMAT0019951 | 1.724 | 5.79E−15 | SEQ ID NO: 448 |
| hsa-miR-4720-5p | MIMAT0019833 | 1.853 | 5.80E−15 | SEQ ID NO: 449 |
| hsa-miR-10b-5p | MIMAT0000254 | 2.170 | 5.83E−15 | SEQ ID NO: 450 |
| hsa-miR-1292-3p | MIMAT0022948 | 0.889 | 5.85E−15 | SEQ ID NO: 451 |
| hsa-let-7e-5p | MIMAT0000066 | 2.241 | 6.02E−15 | SEQ ID NO: 452 |
| hsa-miR-4775 | MIMAT0019931 | 1.564 | 6.22E−15 | SEQ ID NO: 453 |
| hsa-miR-4766-5p | MIMAT0019917 | 2.246 | 7.41E−15 | SEQ ID NO: 454 |
| hsa-miR-370-3p | MIMAT0000722 | 0.939 | 7.61E−15 | SEQ ID NO: 455 |
| hsa-miR-622 | MIMAT0003291 | 1.688 | 8.02E−15 | SEQ ID NO: 456 |
| hsa-miR-4789-3p | MIMAT0019960 | 1.970 | 8.28E−15 | SEQ ID NO: 457 |
| hsa-miR-100-5p | MIMAT0000098 | 2.243 | 8.33E−15 | SEQ ID NO: 458 |
| hsa-miR-3121-3p | MIMAT0014983 | 2.056 | 8.80E−15 | SEQ ID NO: 459 |
| hsa-miR-4659b-3p | MIMAT0019734 | 1.758 | 9.37E−15 | SEQ ID NO: 460 |
| hsa-miR-769-5p | MIMAT0003886 | 1.753 | 9.56E−15 | SEQ ID NO: 461 |
| hsa-miR-219a-1-3p | MIMAT0004567 | 1.884 | 9.64E−15 | SEQ ID NO: 462 |
| hsa-miR-3118 | MIMAT0014980 | 1.621 | 9.83E−15 | SEQ ID NO: 463 |
| hsa-miR-6803-5p | MIMAT0027506 | −0.193 | 9.99E−15 | SEQ ID NO: 464 |
| hsa-miR-4731-3p | MIMAT0019854 | 0.797 | 1.00E−14 | SEQ ID NO: 465 |
| hsa-miR-556-5p | MIMAT0003220 | 1.988 | 1.01E−14 | SEQ ID NO: 466 |
| hsa-miR-6743-5p | MIMAT0027387 | 0.312 | 1.09E−14 | SEQ ID NO: 467 |
| hsa-miR-891b | MIMAT0004913 | 2.163 | 1.14E−14 | SEQ ID NO: 468 |
| hsa-miR-628-5p | MIMAT0004809 | 2.113 | 1.15E−14 | SEQ ID NO: 469 |
| hsa-miR-6792-5p | MIMAT0027484 | 0.696 | 1.17E−14 | SEQ ID NO: 470 |
| hsa-miR-29c-3p | MIMAT0000681 | 2.160 | 1.20E−14 | SEQ ID NO: 471 |
| hsa-miR-4722-5p | MIMAT0019836 | 0.738 | 1.22E−14 | SEQ ID NO: 472 |
| hsa-miR-1305 | MIMAT0005893 | 2.238 | 1.25E−14 | SEQ ID NO: 473 |
| hsa-miR-3129-5p | MIMAT0014992 | 1.575 | 1.40E−14 | SEQ ID NO: 474 |
| hsa-miR-3659 | MIMAT0018080 | 1.862 | 1.43E−14 | SEQ ID NO: 475 |
| hsa-miR-3144-3p | MIMAT0015015 | 1.522 | 1.44E−14 | SEQ ID NO: 476 |
| hsa-miR-4688 | MIMAT0019777 | 0.529 | 1.50E−14 | SEQ ID NO: 477 |
| hsa-miR-6825-5p | MIMAT0027550 | −0.965 | 1.54E−14 | SEQ ID NO: 478 |
| hsa-miR-651-3p | MIMAT0026624 | 1.766 | 1.54E−14 | SEQ ID NO: 479 |
| hsa-miR-4707-3p | MIMAT0019808 | 0.587 | 1.57E−14 | SEQ ID NO: 480 |
| hsa-miR-1243 | MIMAT0005894 | 1.940 | 1.59E−14 | SEQ ID NO: 481 |
| hsa-miR-6744-5p | MIMAT0027389 | 1.892 | 1.62E−14 | SEQ ID NO: 482 |
| hsa-miR-6871-3p | MIMAT0027643 | 1.917 | 1.65E−14 | SEQ ID NO: 483 |
| hsa-miR-4420 | MIMAT0018933 | 2.191 | 1.66E−14 | SEQ ID NO: 484 |
| hsa-miR-592 | MIMAT0003260 | 1.989 | 1.69E−14 | SEQ ID NO: 485 |
| hsa-miR-3128 | MIMAT0014991 | 1.702 | 1.70E−14 | SEQ ID NO: 486 |
| hsa-miR-2114-5p | MIMAT0011156 | 1.983 | 1.77E−14 | SEQ ID NO: 487 |
| hsa-miR-4742-3p | MIMAT0019873 | 2.118 | 1.77E−14 | SEQ ID NO: 488 |
| hsa-miR-3671 | MIMAT0018094 | 1.800 | 1.77E−14 | SEQ ID NO: 489 |
| hsa-miR-7855-5p | MIMAT0030430 | 1.847 | 1.83E−14 | SEQ ID NO: 490 |
| hsa-miR-5006-5p | MIMAT0021033 | 1.498 | 1.92E−14 | SEQ ID NO: 491 |
| hsa-miR-4668-5p | MIMAT0019745 | 1.949 | 1.97E−14 | SEQ ID NO: 492 |
| hsa-miR-3925-5p | MIMAT0018200 | 1.380 | 2.01E−14 | SEQ ID NO: 493 |
| hsa-miR-3658 | MIMAT0018078 | 1.924 | 2.07E−14 | SEQ ID NO: 494 |
| hsa-miR-6756-5p | MIMAT0027412 | 0.445 | 2.11E−14 | SEQ ID NO: 495 |
| hsa-miR-548aw | MIMAT0022471 | 1.631 | 2.20E−14 | SEQ ID NO: 496 |
| hsa-miR-362-3p | MIMAT0004683 | 2.149 | 2.38E−14 | SEQ ID NO: 497 |
| hsa-miR-6805-5p | MIMAT0027510 | −0.412 | 2.40E−14 | SEQ ID NO: 498 |
| hsa-miR-4762-3p | MIMAT0019911 | 1.748 | 2.44E−14 | SEQ ID NO: 499 |
| hsa-miR-6873-5p | MIMAT0027646 | 2.204 | 2.53E−14 | SEQ ID NO: 500 |

### 3.2. Development of Diagnostic Model

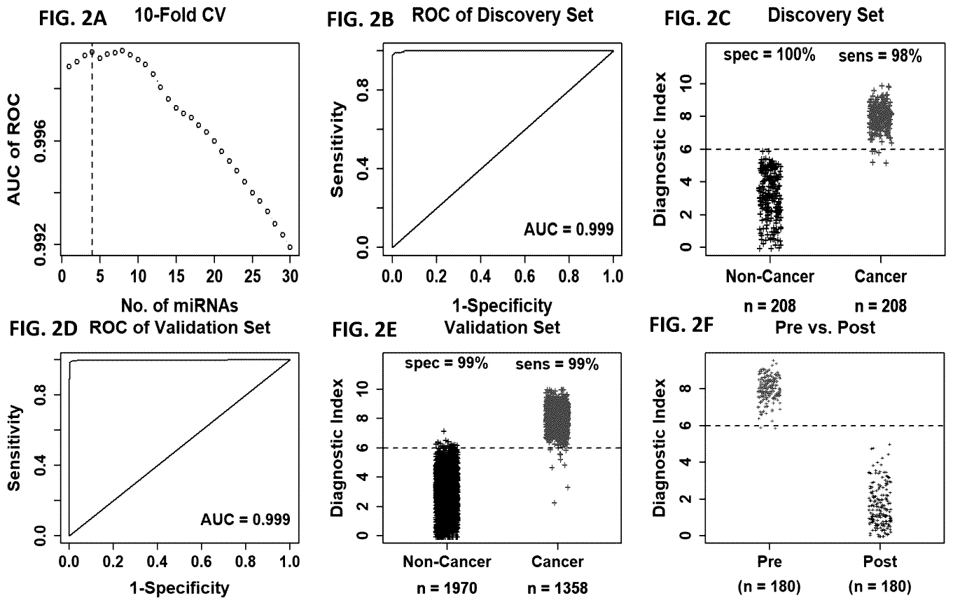

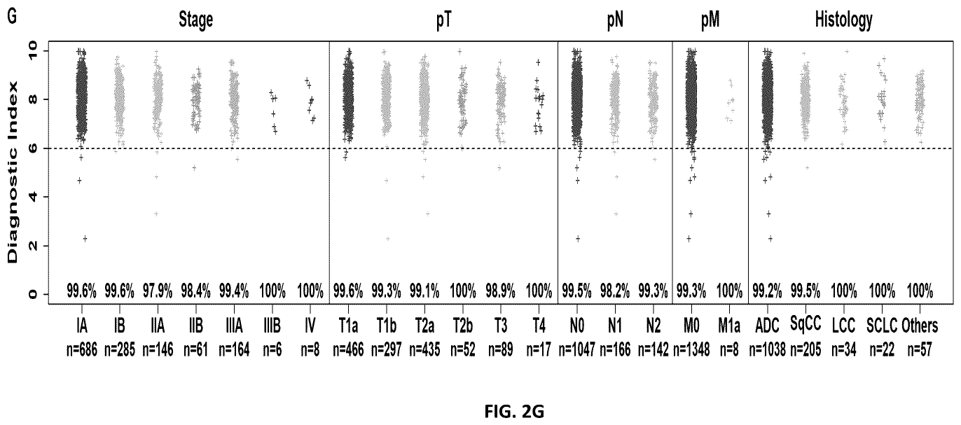

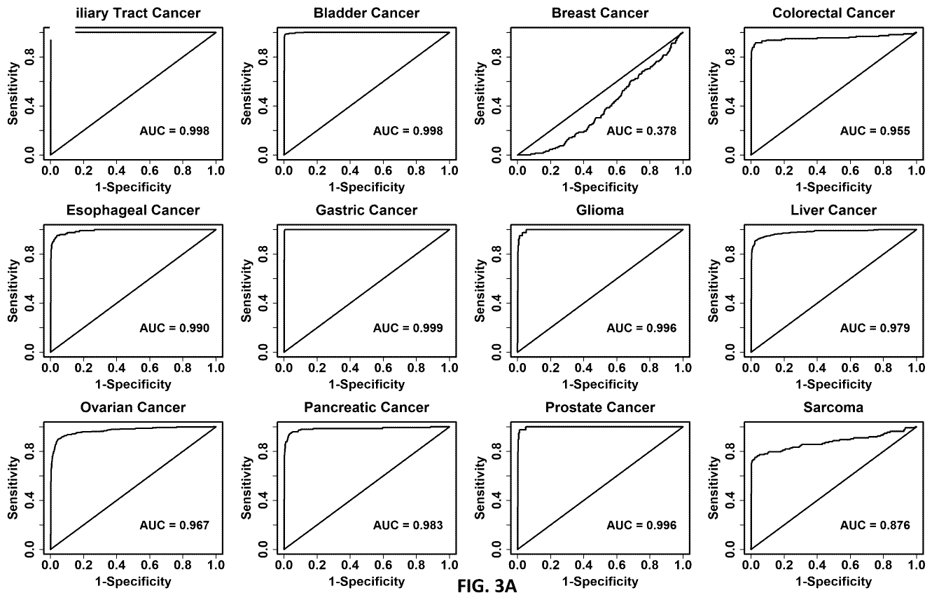

Diagnostic model development was performed in the discovery set of the lung cancer dataset, which included 208 lung cancer patients and 208 non-cancer controls (FIG. 1A). limma analysis was used to evaluate the statistical significance of differential miRNA expression between lung cancer patients and non-cancer controls. The top 500 differentially expressed miRNAs were listed in Table 1. Ten-fold cross validation showed that a diagnostic model with the top 4 miRNAs ranked by adjusted p values (hsa-miR-5100, hsa-miR-1343-3p, hsa-miR-1290, and hsa-miR-4787-3p) would result in the best AUC in the ROC curve analysis (FIG. 2A). A diagnostic index calculated by the weighted sum of the 4 miRNA expression levels and normalized to the range of zero to ten showed a near-perfect AUC value of 0.999 (FIG. 2B), numerically better than the AUC of 0.993 for the original 2-miRNA model from the original publication (Asakura et al. 2020) (p=0.16). The cut-point of six was chosen to ensure no misclassification of the non-cancer controls in the discovery set to minimize the false positives, which resulted in 98% sensitivity and 100% specificity (FIG. 2C), compared to 99% for both sensitivity and specificity for the original 2-miRNA model (Asakura et al. 2020).

### 3.3. Validation of the Diagnostic Model in the Lung Cancer Validation Set

The performance of the 4-miRNA model was evaluated in the lung cancer validation set (n=3328), including 1358 lung cancer patients and 1970 non-cancer controls. The 4-miRNA model achieved an AUC of 0.999 (FIG. 2D), significantly better than the AUC of 0.996 for the original 2-miRNA model (Asakura et al. 2020) (p=0.01). The new model also resulted in 99% for both sensitivity and specificity (FIG. 2E), whereas the original 2-miRNA model showed 95% sensitivity and 99% specificity (Asakura et al. 2020).

Furthermore, the performance of the 4-miRNA model was assessed in clinical subsets of the validation set, as defined by clinical stage, T stage, N stage, M stage, and Histology. Across all clinical subsets, the 4-miRNA model showed sensitivities of approximately 99% or above (FIG. 2G, Table 2), which were superior to the sensitivities of the original 2-miRNA model (Table 2). In particular for early stage lung cancer, e.g., for both patients with stage I lung cancer and patients with T1 tumors, the 4-miRNA model demonstrated >99% sensitivity (FIG. 2G, Table 2), compared to the sensitivities of 95.4 and 95.9%, respectively, for the 2-miRNA model (Table 2). In the prevalent histological types of adenocarcinoma and squamous cell carcinoma, the 4-miRNA model also demonstrated superior performance (FIG. 2G, Table 2), compared to the original 2-miRNA model (Table 2).

TABLE 2
Comparison of sensitivities in the clinical subsets of the lung
cancer validation set between the original 2-miRNA model and the
new 4-miRNA model, while maintaining a specificity of >99%.

| | | | Original 2- | New 4- | |
|---|---|---|---|---|---|
| Clinical Subsets | | N | miRNA model | miRNA model | P value* |
| Clinical | IA | 686 | 96.1% | 99.6% | <0.001 |
| Stage | IB | 285 | 93.7% | 99.6% | <0.001 |
| | IIA | 146 | 97.3% | 97.9% | 0.99 |
| | IIB | 61 | 96.7% | 98.4% | 0.99 |
| | IIIA | 164 | 90.2% | 99.4% | <0.001 |
| | IIIB | 6 | 83.3% | 100.0% | 0.99 |
| | IV | 8 | 100.0% | 100.0% | 1.00 |
| T Stage | T1a | 466 | 96.1% | 99.6% | <0.001 |
| | T1b | 297 | 95.6% | 99.3% | 0.003 |
| | T2a | 435 | 93.6% | 99.1% | <0.001 |
| | T2b | 52 | 92.3% | 100.0% | 0.134 |
| | T3 | 89 | 94.4% | 98.9% | 0.221 |
| | T4 | 17 | 94.1% | 100.0% | 0.99 |
| N Stage | N0 | 1047 | 95.5% | 99.5% | <0.001 |
| | N1 | 166 | 95.8% | 98.2% | 0.289 |
| | N2 | 142 | 90.1% | 99.3% | <0.001 |
| M Stage | M0 | 1348 | 94.7% | 99.3% | <0.001 |
| | M1a | 8 | 100.0% | 100.0% | 1.00 |
| Histology | ADC | 1038 | 95.1% | 99.2% | <0.001 |
| | SqCC | 205 | 94.2% | 99.5% | 0.006 |
| | LCC | 34 | 97.1% | 100.0% | 0.99 |
| | SCLC | 22 | 90.9% | 100.0% | 0.480 |
| | Others | 57 | 96.5% | 100.0% | 0.480 |

*p values calculated by McNemar Test.

Data on paired serum samples (pre- vs. post-surgery) were also available for 180 patients. The diagnostic indices of the 4-miRNA model for post-surgery serum samples were reduced to normal levels below the diagnostic index cut-point (FIG. 2F).

### 3.4. Application of the Diagnostic Model in Additional Cancer Types

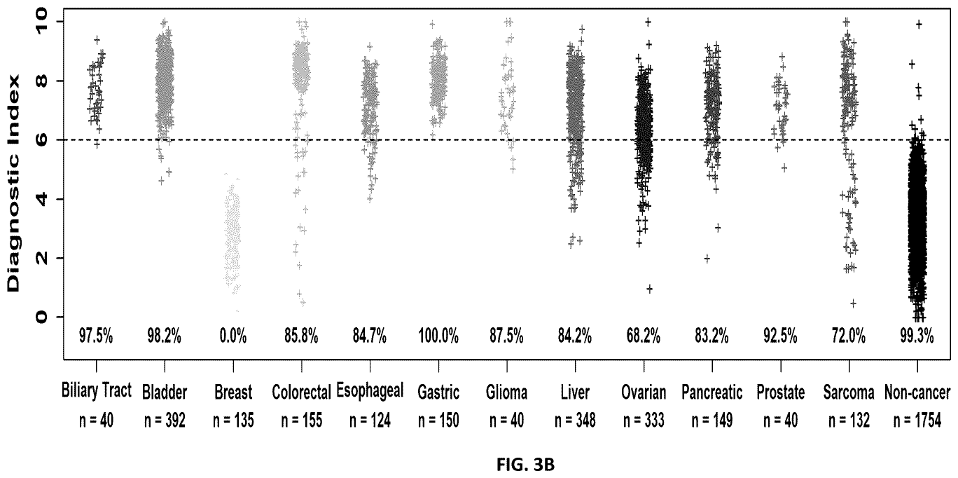

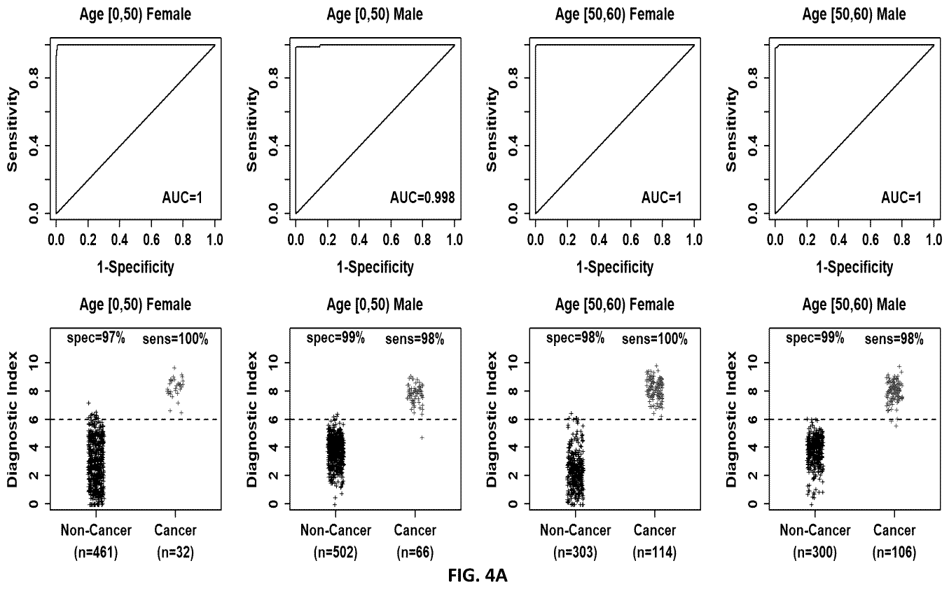

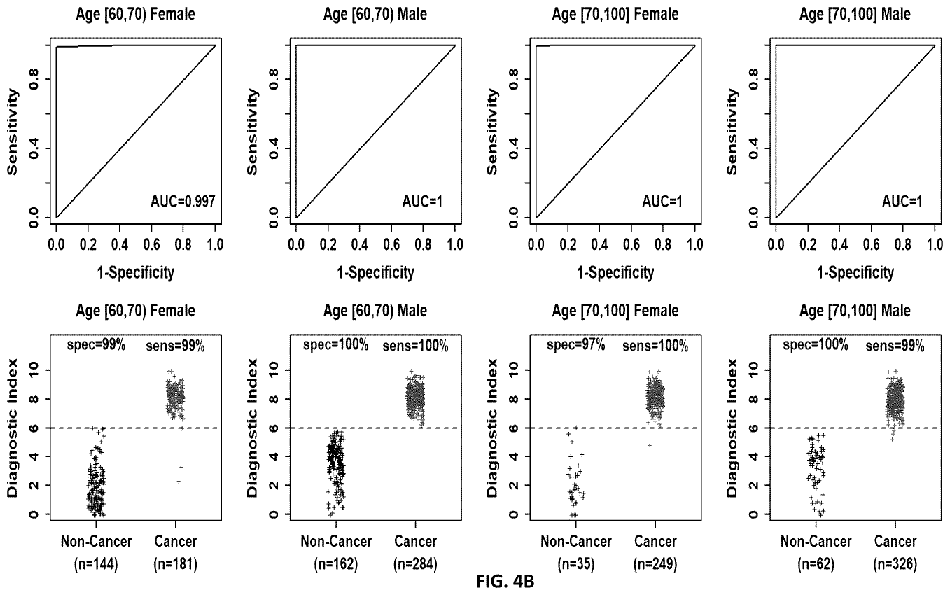

The performance of the 4-miRNA model was further assessed in the combined dataset of 3792 patients, including 2038 cancer patients across 12 different cancer types and 1754 non-cancer controls. The bladder, liver, and ovarian cancers had the largest sample sizes with >300 patients in each. Except for breast cancer in which the 4-miRNA model did not perform, the 4-miRNA model showed very strong performances with AUCs>0.95 in biliary tract, bladder, colorectal, esophageal, gastric, glioma, liver, ovarian, pancreatic, and prostate cancers, and an AUC of 0.876 in Sarcoma (FIG. 3A). Accordingly, the 4-miRNA model demonstrated high sensitivities in the range from 83.2 to 100% for biliary tract, bladder, colorectal, esophageal, gastric, glioma, liver, pancreatic, and prostate cancers, and reasonable sensitivities of 68.2 and 72.0% for ovarian cancer and sarcoma, respectively (FIG. 3B). In addition, for the 1754 non-cancer controls independent of those included in the lung cancer dataset, the 4-miRNA model maintained a high specificity of 99.3%.

A further sensitivity analysis with an alternative diagnostic index cut-point of 5.1 that would lower the specificity to 95% resulted in increased sensitivities across all 11 cancer types, demonstrating sensitivities of >90% across ten cancer types with the exception of 76.5% sensitivity for sarcoma (Table 3).

TABLE 3
Comparison of sensitivities of the 4-miRNA diagnostic model
in additional cancer datasets based on the default cut-point
vs. alternative cut-point that resulted in 95% specificity.

| | Default cut-point | Alternative cut-point |
|---|---|---|
| | based on 99% specificity | based on 95% specificity |
| Biliary Tract Cancer | 97.5% | 100.0% |
| Bladder Cancer | 98.2% | 99.2% |
| Colorectal Cancer | 85.8% | 91.6% |
| Esophageal Cancer | 84.7% | 95.2% |
| Gastric Cancer | 100.0% | 100.0% |
| Glioma | 87.5% | 97.5% |
| Liver Cancer | 84.2% | 92.5% |
| Ovarian Cancer | 68.2% | 90.1% |
| Pancreatic Cancer | 83.2% | 95.3% |
| Prostate Cancer | 92.5% | 97.5% |
| Sarcoma | 72.0% | 76.5% |

### 4. Discussion

In this example, we report on the development and performance evaluation of a 4-miRNA diagnostic model for multi-cancer early detection. We demonstrated that in the large independent validation set of 7120 participants including 3396 cancer patients and 3724 non-cancer individuals, the 4-miRNA model can detect 12 cancer types (biliary tract, bladder, colorectal, esophageal, gastric, glioma, live, lung, ovarian, pancreatic, prostate, and sarcoma) simultaneously with high sensitivities (80-100% for ten cancer types, and ˜70% for two cancer types) while still maintaining a very high specificity of 99% that is typically required for a screening test to be useful in at-risk general population. To our knowledge, this is the first MCED diagnostic model based on circulating cell-free miRNAs. It is interesting to note that the diagnostic index for lung cancer patients decreased to the levels of non-cancer controls after tumor resection, suggesting that the diagnostic model might have the potential for monitoring tumor recurrence.

Noninvasive screening tests analyzing circulating nucleic acids and/or proteins have become the driving force of the MCED campaign with significant progress being made recently. Nearly all of the tests that are being developed for MCED are based on the evaluation of circulating tumor DNAs, and most utilize next generation bisulfite sequencing technology to evaluate the methylation patterns of these tumor DNAs (Klein et al. 2021; Cohen et al. 2018; Chen et al. 2020; and Cristiano et al. 2019). Two such tests, Galleri and PanSeer, are developed as methylation-based epigenetic signatures (Klein et al. 2021; Chen 2020). In the analysis of the case-control study of the Circulating Cell-free Genome Atlas (CCGA), Galleri interrogated >100,000 methylated regions and showed that the sensitivity for 12 pre-specified cancers (anus, bladder, colon/rectum, esophagus, head and neck, liver/bile-duct, lung, lymphoma, ovary, pancreas, plasma cell neoplasm, stomach) was 67.6% for patients with stage I-III disease (n=874) and increased to 76.3% (n=1346) when stage IV cancer was included, while reaching a 99.3% specificity based on 1254 non-cancer controls (Klein et al. 2021). On the other hand, PanSeer assay which targeted only 477 methylated genomic regions retrospectively analyzed plasma samples from a group of asymptomatic individuals enrolled in a longitudinal cancer monitoring study, and demonstrated a high sensitivity of 95% in 98 individuals who later were diagnosed with one of five cancers (stomach, esophageal, colorectal, lung, and liver cancer) within four years of blood draw (pre-diagnosis samples), but with a lower specificity of 96% in 207 healthy controls (Chen et al. 2020). However, what was puzzling with PanSeer was that when it was evaluated in 113 post-diagnosis plasma samples, the test only showed a lower 88% sensitivity (Chen et al. 2020). Another test called DELFI, based on the genome-wide analysis of cell-free DNA fragmentation patterns by next generation sequencing, achieved a 73% sensitivity across seven cancers (n=208, breast, bile duct, colorectal, gastric, lung, ovarian, and pancreatic) and 98% specificity (n=215) (Cristiano et al. 2019). Finally, CancerSEEK, a test combining the measurement of nine protein biomarkers and detection of mutations of 16 genes in circulating cell-free DNA, showed ten-fold cross-validations and median 70% sensitivity (n=1005) across eight cancers (n=1005, ovary, liver, stomach, pancreas, esophagus, colorectum, lung, and breast) and 99% specificity (n=812) (Cohen et al. 2018). In summary, the current MCED tests in development generally showed sensitivities in the range of 60-70% when a high specificity of 99% was mandated. Compared to these tests, our diagnostic model was much simpler with only 4 miRNAs and yet demonstrated substantially higher sensitivities in the range of 80-100% for 10 out of 12 cancer types studied with a large cohort of over 7000 participants. It is worthy of note that a simple diagnostic model not only costs significantly lower, but also can be developed into an in vitro diagnostic (IVD) test using conventional technology platform such as RT-PCR capable of decentralized testing, which has an advantage over NGS-based tests that are usually implemented as a laboratory developed test (LDT). These characteristics are important to drive the wide adoption and compliance of MCED tests as they are intended to target high-risk or at-risk general public.

Among the 13 cancer types examined in this study, only breast cancer was not detected successfully by the 4-miRNA diagnostic model. While the reason for this underperformance was not clear, it may indicate that breast cancer has a different miRNA expression profile and/or different shedding pattern of miRNAs into the bloodstream. Interestingly, Galleri and CancerSEEK also showed poor sensitivity of 30.5 and 33% in breast cancer, respectively (Klein et al. 2021; Cohen et al. 2018). Nevertheless, the poor performance in breast cancer may not be clinically important because mammography screening has been very effective in detecting early stage breast cancer and decreasing breast cancer mortality (Nelson et al. 2016).

The ultimate diagnostic performance and clinical value of these MCED tests has to be established in large prospective screening trials with asymptomatic individuals. In the DETECT-A trial enrolling more than 10,000 asymptomatic women, 96 cancers were identified across ten cancer types, CancerSEEK showed a sensitivity of 27%, and that increased to 52% when adding those detected by standard-of-care screening tests (Lennon et al. 2020). In addition, CancerSEEK, when combined by PET-CT scan, showed a specificity of 99.6% and a positive predictive value (PPV) of 40.6%. On the other hand, in the interim analysis of 4033 participants from the prospective PATHFINDER study of Galleri test, 40 had a positive test result, 18 of them were confirmed to have cancer leading to a PPV of 45% (Beer et al. 2021). For our 4-miRNA diagnostic model, assuming 1% cancer incidence rate and a conservative average sensitivity of 85 and 99.3% specificity, our model would provide a PPV of 55% when screening asymptomatic individuals. This is significantly higher than the PPVs for the four USPSTF recommended single cancer screenings, which range from 3.7 to 4.4% (Lehman et al. 2017; U.S. Food and Drug Administration Cologuard Summary of Safety and Effectiveness Data, 2014; and National Lung Screening Trial Research Team, 2013).

### 5. Conclusions

In summary, our study has provided proof-of-concept data for a simple and affordable blood-based diagnostic test that detects multiple cancers. The 12 cancer types that were detected in this study account for almost 380,000 (˜62%) estimated cancer deaths in the US in 2021. While the early detection of these cancers should conceivably reduce the cancer-related deaths, the ultimate determination of clinical performance and clinical utility will require the evaluation in large prospective studies with asymptomatic individuals from the intended use population.

It is noted that the although the examples and data provided above only cover 12 cancers, for which the miRNA biomarker set, especially the 4-miRNA biomarker set, has demonstrated excellent power in the detection of cancers with very high accuracy, there is no limitation to the cancer types that the miRNA biomarker set can be applied. Accordingly, the scope of the present disclosure shall be interpreted to cover other cancer types as well. The fact that the model provided in the present disclosure works for 12 of the 13 cancer types studied strongly suggests that the method is applicable to most, if not all of the cancer types.

### Example 2

In this example, three datasets were used, namely lung cancer training set, lung cancer testing set, validation set 1, and validation set 2. The first three datasets correspond to the lung cancer discovery set, the lung cancer validation set, and the combined dataset in the original filing. The last dataset, validation set 2, was derived from the following four studies: Sudo K. et al. JAMA Network Open 2019; 2(5):e194573; Abe S. et al. Gastric Cancer 2021; 24(4):835; Ohno M. et al. JAMA Network Open 2019; 2(12):e1916953; Urabe F. et al. Clinical Cancer Research 2019; 25(10):3016. They contain more samples from the four cancer types studied as shown in the figures.

This example is to evaluate whether the diagnostic model provided in this present disclosure that comprises any number (1, 2, 3, . . . , or 500) of miRNAs that are randomly selected from the top 500 miRNAs identified by the limma analysis in the lung cancer training set (as listed in Table 1) in detecting multiple cancer types beyond lung cancer, including biliary tract cancer, bladder cancer, colorectal cancer, esophageal cancer, gastric cancer, glioma, liver cancer, ovarian cancer, pancreatic cancer, prostate cancer, and sarcoma.

Briefly, performance of diagnostic models with varying numbers of miRNAs was evaluated in the lung cancer training set (by 10-fold cross validation), in the lung cancer testing set, and in additional validation sets of other cancer types.

The lung cancer training set included 208 lung cancer patients and 208 non-cancer controls, the lung cancer testing set included 1358 lung cancer patients and 1970 non-cancer controls. The validation set of other cancer types included 2038 cancer patients from 12 cancer types and 1754 non-cancer controls.

More specific description is provided herein.

First, models of 1 miRNA were built for each of the 500 miRNAs. Then, for each of the other numbers of miRNAs (2, 3, 4, . . . , 500), miRNAs were chosen randomly from the top 500 miRNAs for 500 times to build 500 diagnostic models. The performance of these diagnostic models with varying numbers of miRNAs was assessed by AUC of ROC analysis. The boxplots represented the performance of these 500 diagnostic models for each number of miRNAs. The lower end of the box is the 25 percentile, the middle line is the 50 percentile or known as the median, and the upper end of the box is the 75 percentile. In this disclosure, wherever applicable when describing the AUC values using at least one miRNA, it is the median value, i.e., the value at the 50 percentile, that is used.

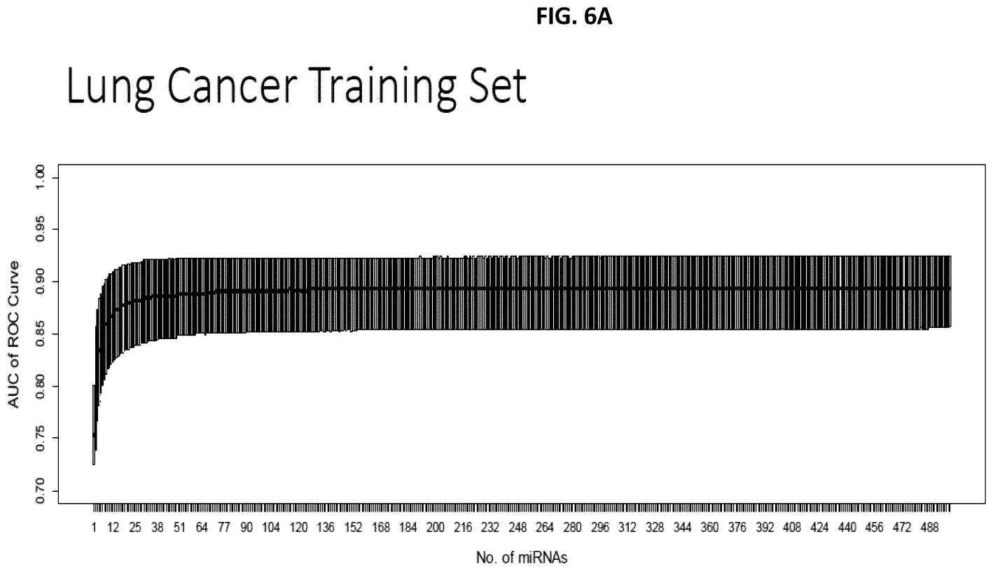

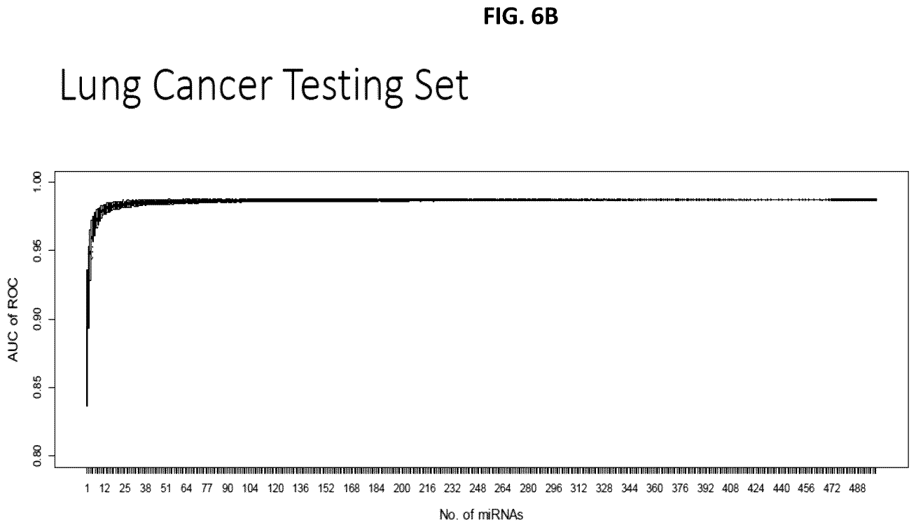

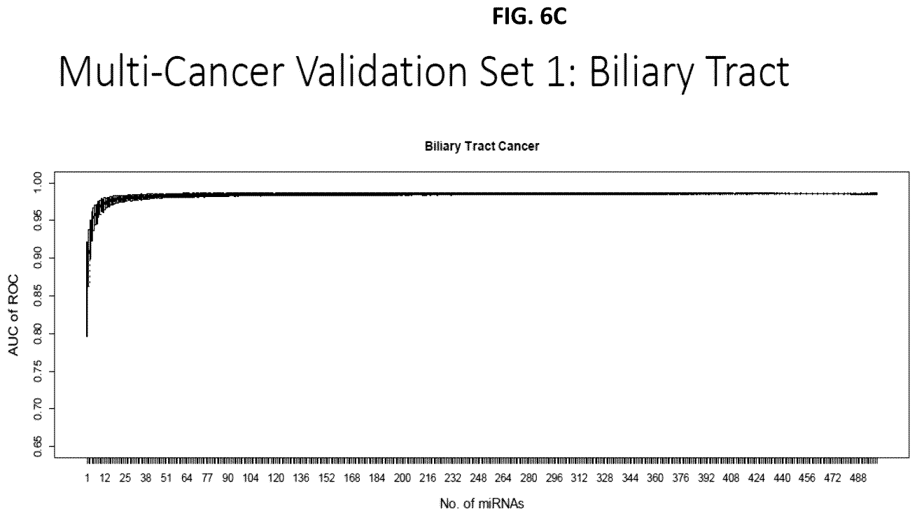

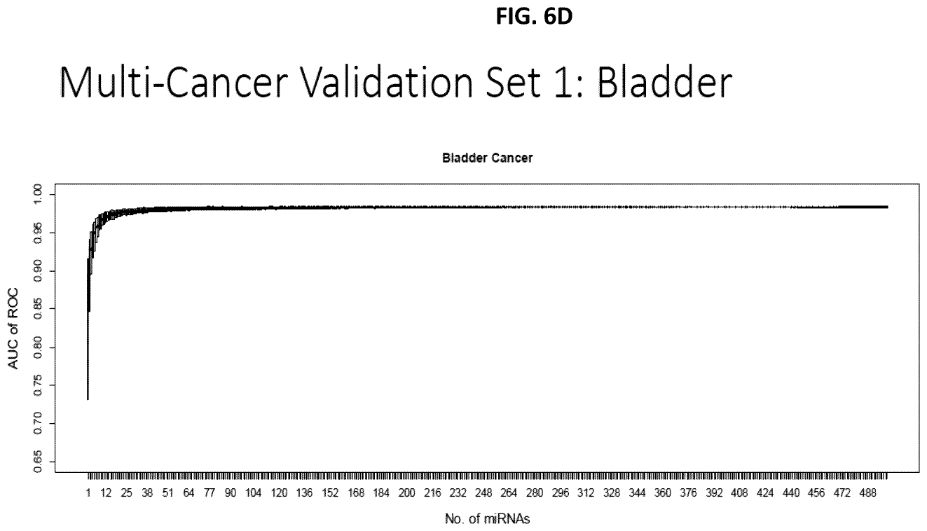

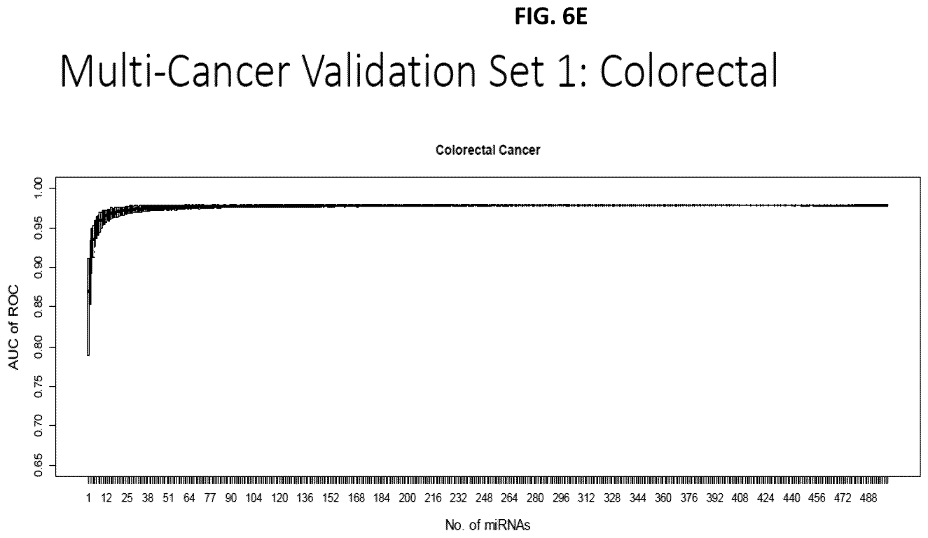

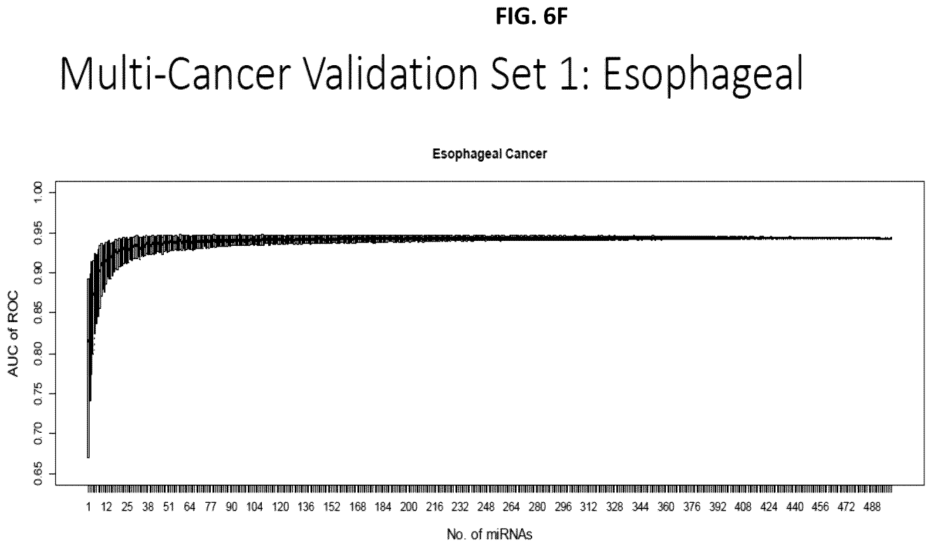

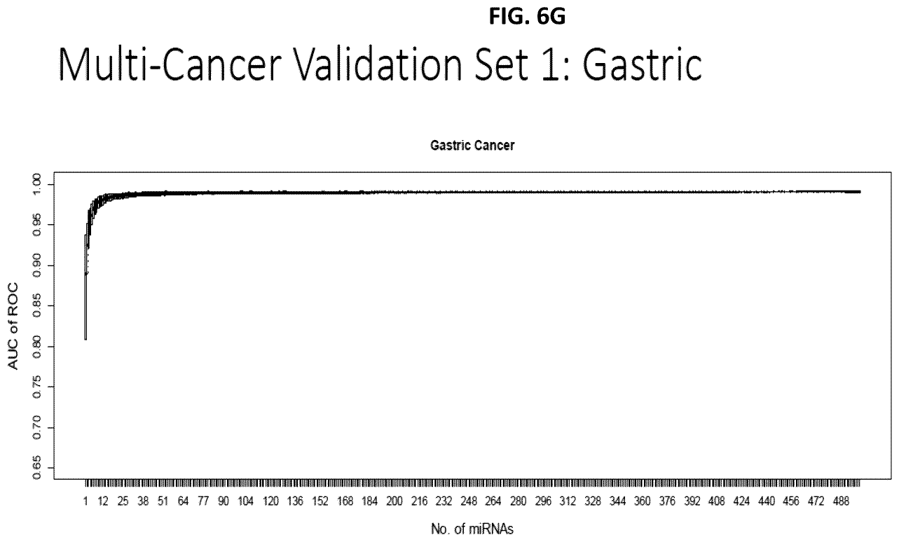

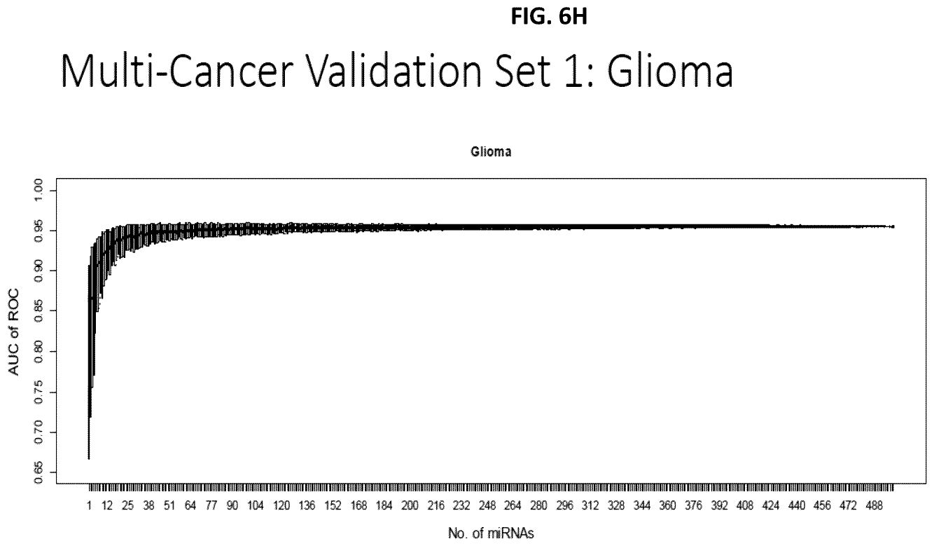

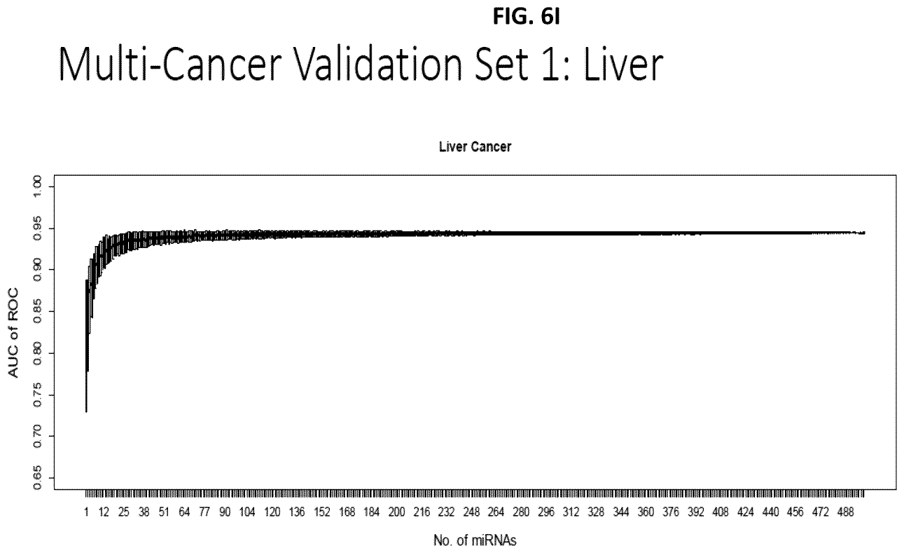

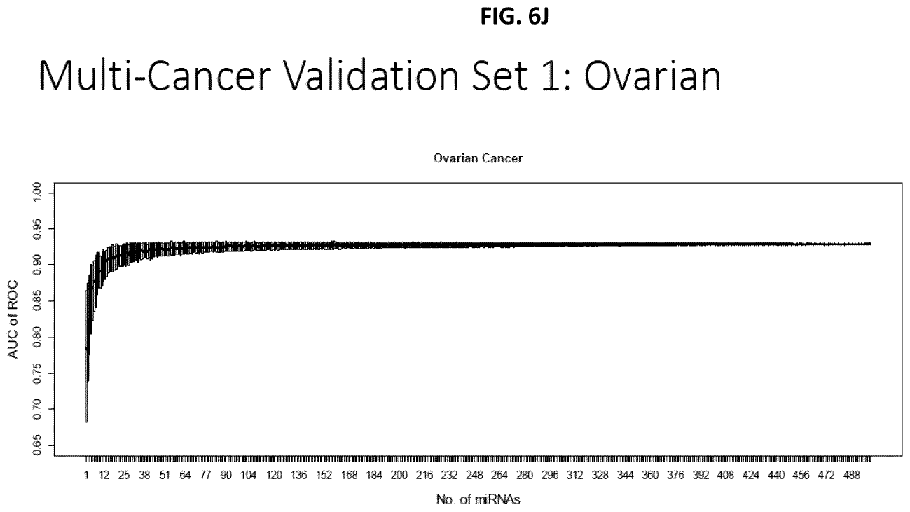

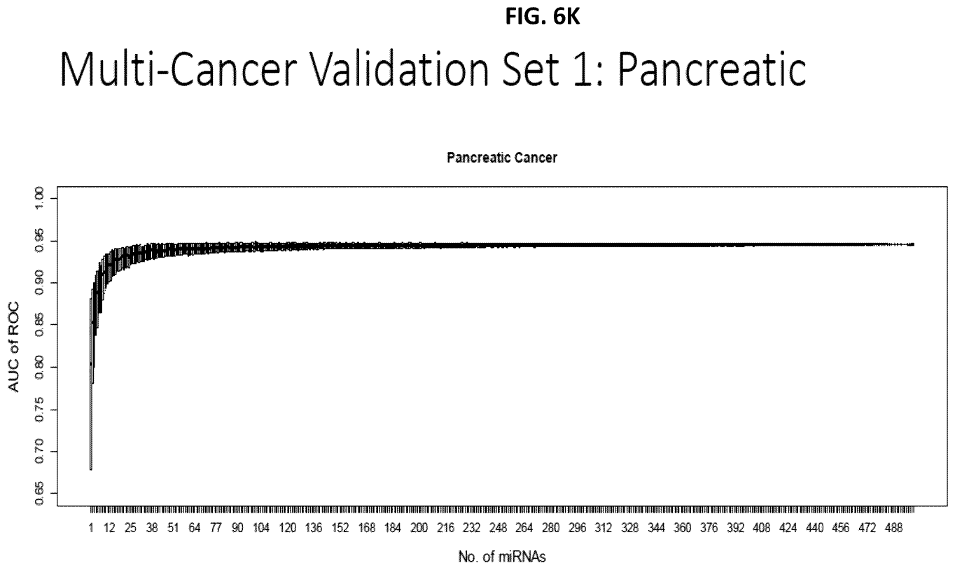

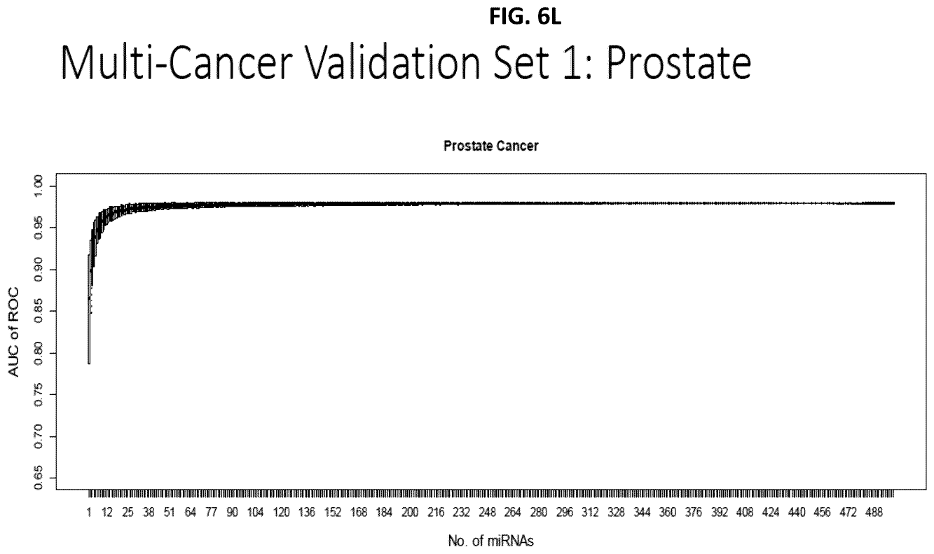

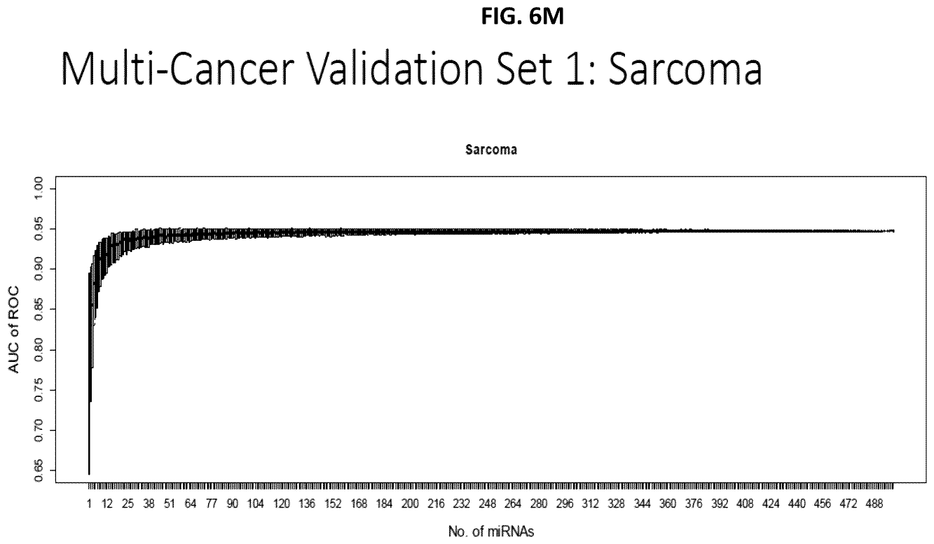

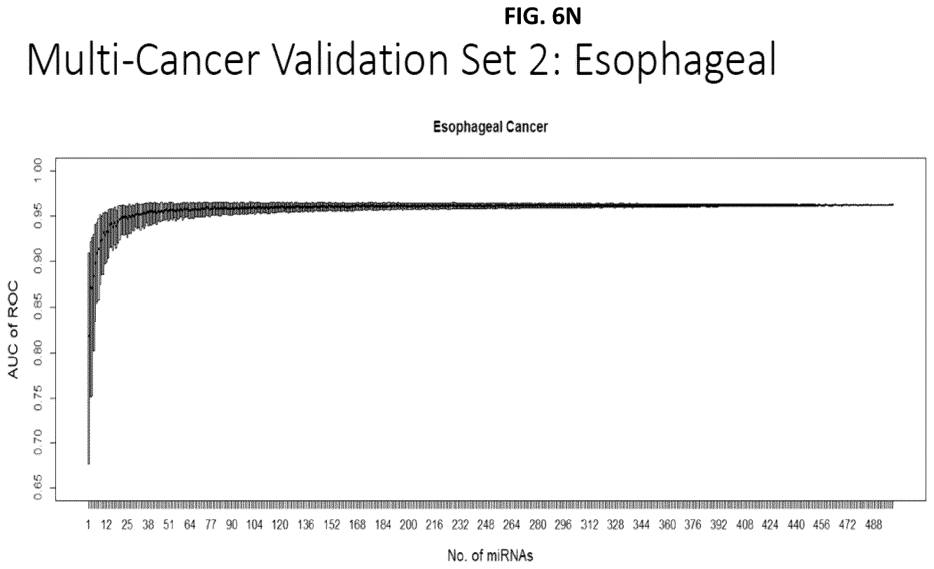

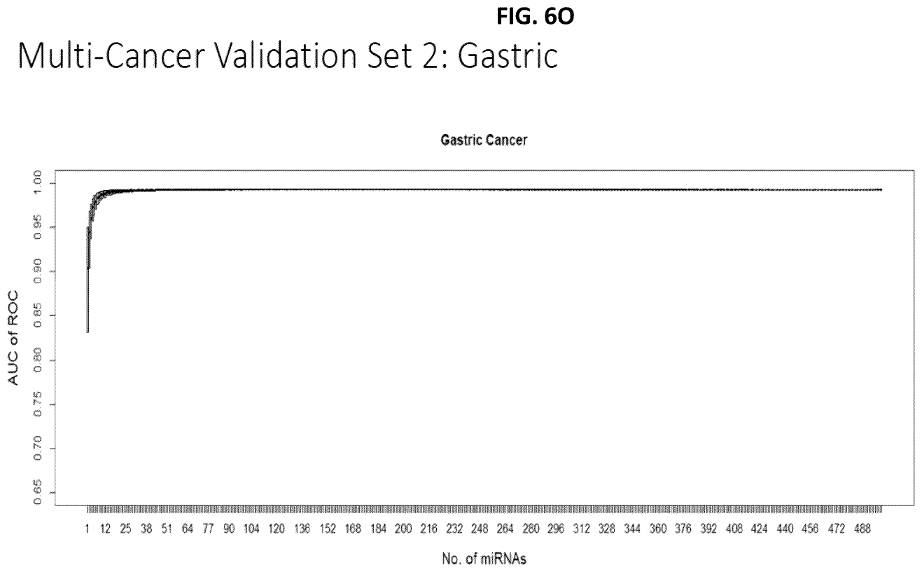

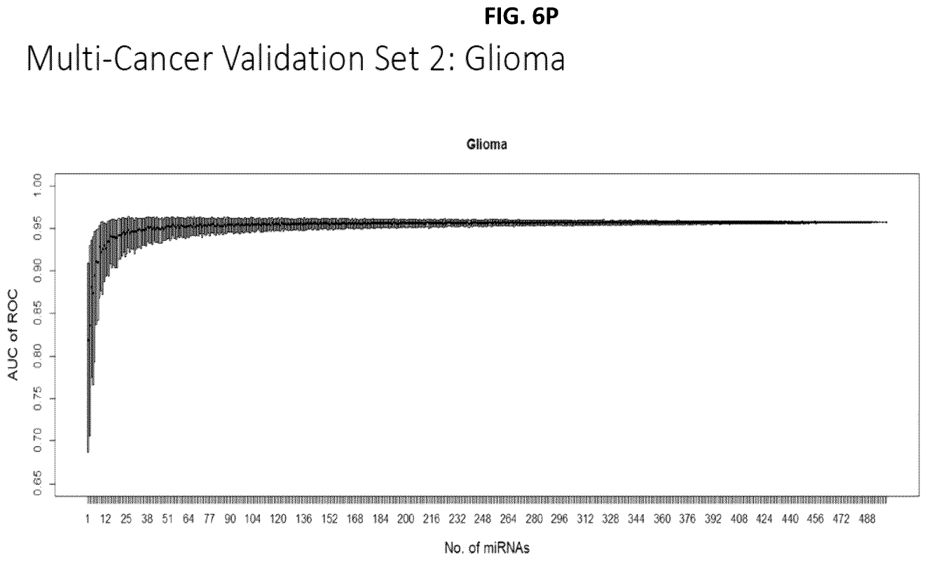

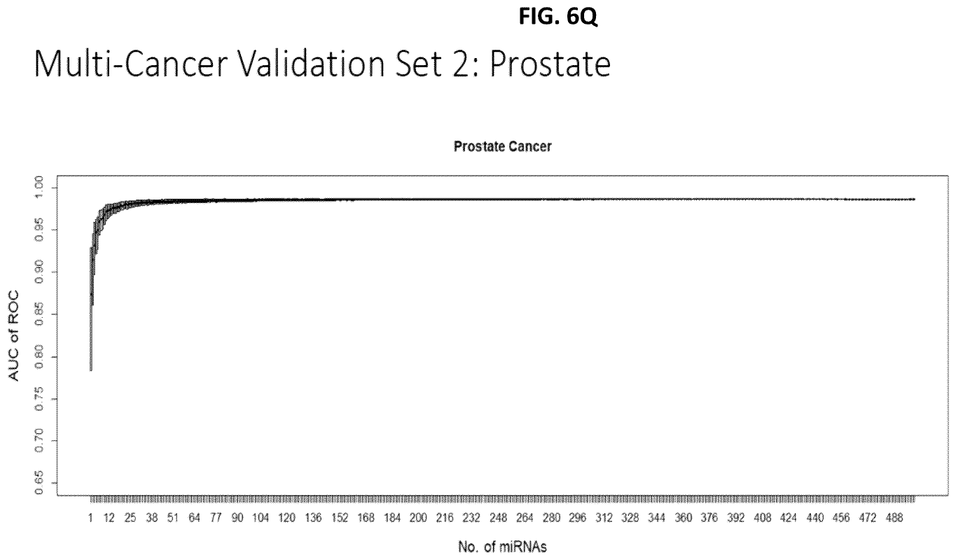

As demonstrated by the results in FIGS. 6A-6Q, a diagnostic model with at least 1 miRNA, including any single one miRNA (n=1), any two miRNAs (n=2), any three miRNAs (n=3), . . . , from the top 500 miRNAs listed in Table 1 can achieve very good performance. The results using the lung cancer training set (from which the 500 miRNAs were derived) is shown as FIG. 6A for reference. Results using the lung cancer testing set are shown in FIG. 6B. FIGS. 6C-6M shows results using validation set 1 for other cancer types. Results using validation set 2 that contains more samples of the 4 cancer type studied, shown in FIGS. 6N-Q, provide additional validation.

### REFERENCES

Ritchie, M E; et al. (2015). limma powers differential expression analyses for RNA-sequencing and microarray studies. Nucleic Acids Research 43(7), e47.
Venables, W N and Ripley, B D (2002) Modern Applied Statistics with S. Fourth edition. Springer.
Tibshirani, R (1996). “Regression Shrinkage and Selection via the lasso”. Journal of the Royal Statistical Society. Series B (methodological). Wiley. 58 (1): 267-88.
Hoerl, A E and Kennard, R W (1970). “Ridge Regression: Biased Estimation for Nonorthogonal problems”. Technometrics. 12 (1): 55-67.
Ripley, B D (1996) Pattern Recognition and Neural Networks. Cambridge University Press.
Kozomara, A and Griffiths-Jones, S (2010). “MiRBase: integrating microRNA annotation and deep-sequencing data”. Nucleic Acids Research. 39 (Database issue): D152-7.
miRBase: the microRNA database: http://www.mirbase.org/The
Bioconductor Open Source Software For Bioinformatics: http://www.bioconductor.org
The R Project for Statistical Computing: https://www.r-project.org/
Asakura, K; et al. (2020). A MiRNA-Based Diagnostic Model Predicts Resectable Lung Cancer in Humans with High Accuracy. Commun. Biol. 3, 134.
Yokoi, A; et al. (2018). Integrated Extracellular MicroRNA Profiling for Ovarian Cancer Screening. Nat. Commun. 9, 4319.
Usuba, W; et al. (2019). Circulating MiRNA Panels for Specific and Early Detection in Bladder Cancer. Cancer Sci. 110, 408-419.
Yamamoto, Y; et al. (2020). Highly Sensitive Circulating MicroRNA Panel for Accurate Detection of Hepatocellular Carcinoma in Patients With Liver Disease. Hepatol. Commun. 4, 284-297.
Klein, E A; et al. (2021). Clinical Validation of a Targeted Methylation-Based Multi-Cancer Early Detection Test Using an Independent Validation Set. Ann. Oncol.: Off. J. Eur. Soc. Med. Oncol. 32, 1167-1177.
Cohen, J D; et al. (2018). Detection and Localization of Surgically Resectable Cancers with a Multi-Analyte Blood Test. Science. 359, 926-930.
Chen, X; et al. (2020). Non-Invasive Early Detection of Cancer Four Years before Conventional Diagnosis Using a Blood Test. Nat. Commun. 11, 3475.
Cristiano, S; et al. (2019). Genome-Wide Cell-Free DNA Fragmentation in Patients with Cancer. Nature. 570, 385-389.
Nelson, H D; et al. (2016). Effectiveness of Breast Cancer Screening: Systematic Review and Meta-Analysis to Update the 2009 U.S. Preventive Services Task Force Recommendation. Ann. Intern. Med. 164, 244-255.
Lennon, A M; et al. (2020). Feasibility of Blood Testing Combined with PET-CT to Screen for Cancer and Guide Intervention. Science. 369, eabb9601.
Beer, T; et al. (2021). Interim Results of PATHFINDER, a Clinical Use Study Using a Methylation-Based Multi-Cancer Early Detection Test. J. Clin. Oncol. 39, 3010.
Lehman, C D; et al. (2017). National Performance Benchmarks for Modern Screening Digital Mammography: Update from the Breast Cancer Surveillance Consortium. Radiology. 283, 49-58.
U.S. Food and Drug Administration Cologuard Summary of Safety and Effectiveness Data (Premarket Approval Application P130017); 2014.
National Lung Screening Trial Research Team; Church, T R; et al. (2013). Results of Initial Low-Dose Computed Tomographic Screening for Lung Cancer. New Engl. J. Med. 2013, 368, 1980-1991.
Nielsen, P E; et al. (1991). Sequence-selective recognition of DNA by strand displacement with a thymine-substituted polyamide. Science. 254, p. 1497-500.
Obika, S; et al. (1998). Stability and structural features of the duplexes containing nucleoside analogues with a fixed N-type conformation, 2′-O,4′-C-methyleneribonucleosides. Tetrahedron Lett. 39, p. 5401-5404.
Green, M R and Sambrook, J. (2012). Molecular Cloning: A Laboratory Manual, 4th Ed., Cold Spring Harbor Press, Cold Spring Harbor, N.Y.
Sambrook, J; et al. (1989). Molecular Cloning: A Laboratory Manual, New York: Cold Spring Harbor Press.
Zhang, Z; et al. (2000). A greedy algorithm for aligning DNA sequences. J. Comput. Biol. 7, p. 203-214.
Altschul, S F; et al. (1990). Basic local alignment search tool. Journal of Molecular Biology, Vol. 215, p. 403-410.
Pearson, W R et al. (1988). Improved tools for biological sequence comparison. Proc. Natl. Acad. Sci. U.S.A, Vol. 85, p. 2444-2448.
Yun, S J; et al. (2012). Cell-free microRNAs in urine as diagnostic and prognostic biomarkers of bladder cancer. Int J Oncol. 2012 November; 41(5):1871-8.
Park, N J; et al. (2009). Salivary microRNA: discovery, characterization, and clinical utility for oral cancer detection. Clin Cancer Res. 2009 Sep. 1; 15(17):5473-7.

1. A kit for detecting a cancer from a biological sample obtained from a subject, comprising at least one nucleic acid and at least one instruction, wherein:
each of the at least one nucleic acid is capable of specifically recognizing each miRNA in an miRNA biomarker set, thereby allowing an expression profile of the miRNA biomarker set to be obtained from the biological sample, wherein each miRNA of the miRNA biomarker set is from a 500-miRNA set as listed in Table 1;
the at least one instruction comprises:
a first instruction, comprising a first sub-instruction for calculating a diagnostic index of the biological sample based on the expression profile of the miRNA biomarker set, wherein the diagnostic index is calculated based on formula:
$$diagnostic index = {\sum }_{i = 1}^{n} {t}_{i} * {miRNA}_{i} ; \qquad ( I )$$
where n is the total number of the at least one miRNA in the miRNA biomarker set and is an integer greater than zero and smaller than or equal to 500; miRNAᵢ is the expression level of i^{th} miRNA in the miRNA biomarker set, where i is an integer greater than zero and smaller than or equal to n; and tᵢ is a weight for the i^{th} miRNA; and
a second instruction for classifying the subject as having the cancer or not, wherein the subject is classified as having the cancer if the calculated diagnostic index is greater than or equal to a pre-determined threshold or as not having the cancer if otherwise;
wherein:
the kit is capable of achieving diagnostic accuracy having an AUC value greater than approximately 0.75 for detecting any one of lung cancer, biliary tract cancer, bladder cancer, colorectal cancer, esophageal cancer, gastric cancer, glioma, liver cancer, ovarian cancer, pancreatic cancer, prostate cancer, or sarcoma.

2. The kit of claim 1, wherein in the first sub-instruction of the first instruction, the diagnostic index is calculated via an unweighted model.

3. The kit of claim 1, wherein in the first sub-instruction of the first instruction, the diagnostic index is calculated via a weighted model using weights from one selected from a group consisting of Linear Models for Microarray Data (limma) model, logistic regression model, linear discriminant analysis (LDA) model, conditional logistic regression model, lasso regression model, ridge regression model, random forest, support vector machine, and probit regression model.

4. The kit of claim 3, wherein the diagnostic index is calculated via a weighted model using weights from the limma model.

5. The kit of claim 1, wherein the first instruction further comprises a second sub-instruction for obtaining a normalized diagnostic index based on the diagnostic index calculated according to the first sub-instruction, wherein in the second instruction, the subject is classified as having the cancer if the normalized diagnostic index is greater than or equal to a preset cut-point or as not having the cancer if otherwise.

6. The kit of claim 5, wherein in the second sub-instruction, the normalized diagnostic index is calculated based on formula:
$$normalized diagnostic index = \frac{diagnostic index - {param}_{location}}{{param}_{scale}} ; \qquad ( II )$$
where the param_{location} and param_{scale} are respectively a location parameter and a scale parameter configured to allow the normalized diagnostic index to be within a range no less than a first preset value and no greater than a second preset value.

7. The kit of claim 6, wherein in the first instruction, the diagnostic index is calculated via a weighted model using weights from the limma model, and the first preset value is 0, and the second preset value is 10.

8. The kit of claim 1, wherein the miRNA biomarker set consists of hsa-miR-5100, hsa-miR-1343-3p, hsa-miR-1290, and hsa-miR-4787-3p.

9. The kit of claim 8, wherein the at least one nucleic acid consists of a total of 4 polynucleotides which are respectively capable of specifically hybridizing under a stringent condition to:
(a) polynucleotides respectively comprising or consisting of nucleotide sequences of SEQ ID NOS: 1-4, derivatives thereof, variants thereof each having at least 80% sequence identity, or fragments thereof each comprising 15 or more consecutive nucleotides; or
(b) polynucleotides respectively comprising or consisting of nucleotide sequences which are respectively complementary to nucleotide sequences of SEQ ID NOS: 1-4, derivatives thereof, variants thereof each having at least 80% sequence identity, or fragments thereof each comprising 15 or more consecutive nucleotides.

10. The kit of claim 8, wherein the pre-determined threshold is 1110, and the second instruction further comprises an indication that classification has a specificity value greater than approximately 0.95.

11. The kit of claim 8, wherein the pre-determined threshold is 1200, and the second instruction further comprises an indication that classification has a specificity value greater than approximately 0.99.

12. The kit of claim 8, wherein the first instruction further comprises a second sub-instruction for obtaining a normalized diagnostic index based on the diagnostic index calculated according to the first sub-instruction, wherein in the second sub-instruction, the normalized diagnostic index is calculated based on formula:
$$normalized diagnostic index = \frac{diagnostic index - {param}_{location}}{{param}_{scale}} ; \qquad ( II )$$
where the param_{location} and param_{scale} are respectively a location parameter and a scale parameter configured to allow the normalized diagnostic index to be within a range no less than a first preset value and no greater than a second preset value; wherein in the second instruction, the subject is classified as having the cancer if the normalized diagnostic index is greater than or equal to a preset cut-point or as not having the cancer if otherwise.

13. The kit of claim 12, wherein the preset cut-point is 5.1, and the method is capable of achieving diagnostic accuracy having a specificity value greater than approximately 0.95.

14. The kit of claim 1, wherein the at least one instruction further comprises a third instruction for performing an evaluation of the subject, wherein said evaluation comprises a diagnosis of the cancer or a detection of a recurrence of the cancer.

15. The kit of claim 1, wherein the at least one instruction further comprises a fourth instruction for administering to the subject a therapeutic regimen when the subject is classified as having the cancer.

16. The kit of claim 1, wherein the at least one instruction further comprises a first additional instruction for obtaining the expression profile of the miRNA biomarker set, comprising a procedure for performing Northern Blotting, microarray analysis, RNA-sequencing, or RNA in-situ hybridization by means of the at least one nucleic acid.

17. The kit of claim 16, wherein the at least one nucleic acid is arranged on a molecular array.

18. The kit of claim 1, further comprising at least one set of amplification primers, each set capable of specifically amplifying each of the at least one miRNA in the miRNA biomarker set from the biological sample.

19. The kit of claim 18, wherein the at least one instruction further comprises a second additional instruction for obtaining the expression profile of the miRNA biomarker set, comprising a procedure for performing reverse-transcription PCR (RT-PCR), quantitative RT-PCR (qRT-PCR), or digital RT-PCR by means of the at least one nucleic acid and the at least one set of amplification primers.

20. The kit of claim 1, wherein the biological sample is a liquid biopsy sample selected from a group consisting of a blood sample, a serum sample, a plasma sample, a urine sample, a saliva sample, and a spatum sample.


SEQUENCE LISTING (500 sequences)

SEQ ID NO: 1 - RNA, 22 nt, Homo sapiens
  source 1..22: mol_type = transcribed RNA
ttcagatcccagcggtgcctct

SEQ ID NO: 2 - RNA, 22 nt, Homo sapiens
  source 1..22: mol_type = transcribed RNA
ctcctggggcccgcactctcgc

SEQ ID NO: 3 - RNA, 19 nt, Homo sapiens
  source 1..19: mol_type = transcribed RNA
tggatttttggatcaggga

SEQ ID NO: 4 - RNA, 24 nt, Homo sapiens
  source 1..24: mol_type = transcribed RNA
gatgcgccgcccactgccccgcgc

SEQ ID NO: 5 - RNA, 22 nt, Homo sapiens
  source 1..22: mol_type = transcribed RNA
agggccgaagggtggaagctgc

SEQ ID NO: 6 - RNA, 22 nt, Homo sapiens
  source 1..22: mol_type = transcribed RNA
actgcagtgaaggcacttgtag

SEQ ID NO: 7 - RNA, 25 nt, Homo sapiens
  source 1..25: mol_type = transcribed RNA
gtgaggcggggccaggagggtgtgt

SEQ ID NO: 8 - RNA, 20 nt, Homo sapiens
  source 1..20: mol_type = transcribed RNA
cgggcgtggtggtgggggtg

SEQ ID NO: 9 - RNA, 17 nt, Homo sapiens
  source 1..17: mol_type = transcribed RNA
ccccgccaccgccttgg

SEQ ID NO: 10 - RNA, 22 nt, Homo sapiens
  source 1..22: mol_type = transcribed RNA
aaaccgttaccattactgagtt

SEQ ID NO: 11 - RNA, 21 nt, Homo sapiens
  source 1..21: mol_type = transcribed RNA
gtgggcgggggcaggtgtgtg

SEQ ID NO: 12 - RNA, 22 nt, Homo sapiens
  source 1..22: mol_type = transcribed RNA
acctggcagcagggagcgtcgt

SEQ ID NO: 13 - RNA, 20 nt, Homo sapiens
  source 1..20: mol_type = transcribed RNA
ggatccgagtcacggcacca

SEQ ID NO: 14 - RNA, 22 nt, Homo sapiens
  source 1..22: mol_type = transcribed RNA
ggctacaacacaggacccgggc

SEQ ID NO: 15 - RNA, 17 nt, Homo sapiens
  source 1..17: mol_type = transcribed RNA
accccactcctggtacc

SEQ ID NO: 16 - RNA, 22 nt, Homo sapiens
  source 1..22: mol_type = transcribed RNA
ccgggagaaggaggtggcctgg

SEQ ID NO: 17 - RNA, 22 nt, Homo sapiens
  source 1..22: mol_type = transcribed RNA
ggtggcccggccgtgcctgagg

SEQ ID NO: 18 - RNA, 21 nt, Homo sapiens
  source 1..21: mol_type = transcribed RNA
acggcccaggcggcattggtg

SEQ ID NO: 19 - RNA, 24 nt, Homo sapiens
  source 1..24: mol_type = transcribed RNA
agggctggactcagcggcggagct

SEQ ID NO: 20 - RNA, 24 nt, Homo sapiens
  source 1..24: mol_type = transcribed RNA
gtaggggcgtcccgggcgcgcggg

SEQ ID NO: 21 - RNA, 22 nt, Homo sapiens
  source 1..22: mol_type = transcribed RNA
agactgacggctggaggcccat

SEQ ID NO: 22 - RNA, 23 nt, Homo sapiens
  source 1..23: mol_type = transcribed RNA
tctgggaggttgtagcagtggaa

SEQ ID NO: 23 - RNA, 24 nt, Homo sapiens
  source 1..24: mol_type = transcribed RNA
ccatgaagcagtgggtaggaggac

SEQ ID NO: 24 - RNA, 19 nt, Homo sapiens
  source 1..19: mol_type = transcribed RNA
tgcgcctcgggtgagcatg

SEQ ID NO: 25 - RNA, 18 nt, Homo sapiens
  source 1..18: mol_type = transcribed RNA
cgggcgtggtggtggggg

SEQ ID NO: 26 - RNA, 21 nt, Homo sapiens
  source 1..21: mol_type = transcribed RNA
accactgcactccagcctgag

SEQ ID NO: 27 - RNA, 25 nt, Homo sapiens
  source 1..25: mol_type = transcribed RNA
agcggggaggaagtgggcgctgctt

SEQ ID NO: 28 - RNA, 22 nt, Homo sapiens
  source 1..22: mol_type = transcribed RNA
taaggcacgcggtgaatgccaa

SEQ ID NO: 29 - RNA, 19 nt, Homo sapiens
  source 1..19: mol_type = transcribed RNA
atcccaccactgccaccat

SEQ ID NO: 30 - RNA, 22 nt, Homo sapiens
  source 1..22: mol_type = transcribed RNA
aggactgatcctctcgggcagg

SEQ ID NO: 31 - RNA, 22 nt, Homo sapiens
  source 1..22: mol_type = transcribed RNA
aaaagctgggttgagagggcaa

SEQ ID NO: 32 - RNA, 18 nt, Homo sapiens
  source 1..18: mol_type = transcribed RNA
ttcccagccaacgcacca

SEQ ID NO: 33 - RNA, 23 nt, Homo sapiens
  source 1..23: mol_type = transcribed RNA
tagcaccatttgaaatcagtgtt

SEQ ID NO: 34 - RNA, 22 nt, Homo sapiens
  source 1..22: mol_type = transcribed RNA
agcaaggcggcatctctctgat

SEQ ID NO: 35 - RNA, 21 nt, Homo sapiens
  source 1..21: mol_type = transcribed RNA
ggggggatgtgcatgctggtt

SEQ ID NO: 36 - RNA, 22 nt, Homo sapiens
  source 1..22: mol_type = transcribed RNA
tattgcactcgtcccggcctcc

SEQ ID NO: 37 - RNA, 18 nt, Homo sapiens
  source 1..18: mol_type = transcribed RNA
ccagaggtggggactgag

SEQ ID NO: 38 - RNA, 22 nt, Homo sapiens
  source 1..22: mol_type = transcribed RNA
atagtgggaagctggcagattc

SEQ ID NO: 39 - RNA, 22 nt, Homo sapiens
  source 1..22: mol_type = transcribed RNA
tattgcacttgtcccggcctgt

SEQ ID NO: 40 - RNA, 22 nt, Homo sapiens
  source 1..22: mol_type = transcribed RNA
aggcggggcgccgcgggaccgc

SEQ ID NO: 41 - RNA, 22 nt, Homo sapiens
  source 1..22: mol_type = transcribed RNA
tggcgggggtagagctggctgc

SEQ ID NO: 42 - RNA, 23 nt, Homo sapiens
  source 1..23: mol_type = transcribed RNA
tcgaggactggtggaagggcctt

SEQ ID NO: 43 - RNA, 20 nt, Homo sapiens
  source 1..20: mol_type = transcribed RNA
ctaggtggggggcttgaagc

SEQ ID NO: 44 - RNA, 22 nt, Homo sapiens
  source 1..22: mol_type = transcribed RNA
tggtgggccgcagaacatgtgc

SEQ ID NO: 45 - RNA, 24 nt, Homo sapiens
  source 1..24: mol_type = transcribed RNA
ctgcaggcagaagtggggctgaca

SEQ ID NO: 46 - RNA, 24 nt, Homo sapiens
  source 1..24: mol_type = transcribed RNA
gctggtttcatatggtggtttaga

SEQ ID NO: 47 - RNA, 18 nt, Homo sapiens
  source 1..18: mol_type = transcribed RNA
ggtgggcttcccggaggg

SEQ ID NO: 48 - RNA, 19 nt, Homo sapiens
  source 1..19: mol_type = transcribed RNA
aggcaggttatctgggctg

SEQ ID NO: 49 - RNA, 22 nt, Homo sapiens
  source 1..22: mol_type = transcribed RNA
gcccaggactttgtgcggggtg

SEQ ID NO: 50 - RNA, 18 nt, Homo sapiens
  source 1..18: mol_type = transcribed RNA
gggtgagggcaggtggtt

SEQ ID NO: 51 - RNA, 21 nt, Homo sapiens
  source 1..21: mol_type = transcribed RNA
tcttgaagtcagaacccgcaa

SEQ ID NO: 52 - RNA, 22 nt, Homo sapiens
  source 1..22: mol_type = transcribed RNA
gagggttgggtggaggctctcc

SEQ ID NO: 53 - RNA, 20 nt, Homo sapiens
  source 1..20: mol_type = transcribed RNA
cctgagcccgggccgcgcag

SEQ ID NO: 54 - RNA, 18 nt, Homo sapiens
  source 1..18: mol_type = transcribed RNA
atcctagtcacggcacca

SEQ ID NO: 55 - RNA, 21 nt, Homo sapiens
  source 1..21: mol_type = transcribed RNA
agccaagtggaagttacttta

SEQ ID NO: 56 - RNA, 21 nt, Homo sapiens
  source 1..21: mol_type = transcribed RNA
agccgcggggatcgccgaggg

SEQ ID NO: 57 - RNA, 20 nt, Homo sapiens
  source 1..20: mol_type = transcribed RNA
actcaaactgtgggggcact

SEQ ID NO: 58 - RNA, 21 nt, Homo sapiens
  source 1..21: mol_type = transcribed RNA
agcagacttgacctacaatta

SEQ ID NO: 59 - RNA, 22 nt, Homo sapiens
  source 1..22: mol_type = transcribed RNA
aggcgatgtggggatgtagaga

SEQ ID NO: 60 - RNA, 24 nt, Homo sapiens
  source 1..24: mol_type = transcribed RNA
agcctggaagctggagcctgcagt

SEQ ID NO: 61 - RNA, 19 nt, Homo sapiens
  source 1..19: mol_type = transcribed RNA
aatggatttttggagcagg

SEQ ID NO: 62 - RNA, 23 nt, Homo sapiens
  source 1..23: mol_type = transcribed RNA
atcacattgccagggattaccac

SEQ ID NO: 63 - RNA, 22 nt, Homo sapiens
  source 1..22: mol_type = transcribed RNA
aaaagctgggttgagagggcga

SEQ ID NO: 64 - RNA, 22 nt, Homo sapiens
  source 1..22: mol_type = transcribed RNA
cagccctcctcccgcacccaaa

SEQ ID NO: 65 - RNA, 23 nt, Homo sapiens
  source 1..23: mol_type = transcribed RNA
caacggaatcccaaaagcagctg

SEQ ID NO: 66 - RNA, 20 nt, Homo sapiens
  source 1..20: mol_type = transcribed RNA
aaaagctgggttgagagggt

SEQ ID NO: 67 - RNA, 19 nt, Homo sapiens
  source 1..19: mol_type = transcribed RNA
ggctggtcagatgggagtg

SEQ ID NO: 68 - RNA, 22 nt, Homo sapiens
  source 1..22: mol_type = transcribed RNA
aggactggactcccggcagccc

SEQ ID NO: 69 - RNA, 21 nt, Homo sapiens
  source 1..21: mol_type = transcribed RNA
aggggtgctatctgtgattga

SEQ ID NO: 70 - RNA, 21 nt, Homo sapiens
  source 1..21: mol_type = transcribed RNA
agctgtacctgaaaccaagca

SEQ ID NO: 71 - RNA, 21 nt, Homo sapiens
  source 1..21: mol_type = transcribed RNA
atcacattgccagggatttcc

SEQ ID NO: 72 - RNA, 17 nt, Homo sapiens
  source 1..17: mol_type = transcribed RNA
agggtgtgtgtgttttt

SEQ ID NO: 73 - RNA, 21 nt, Homo sapiens
  source 1..21: mol_type = transcribed RNA
gcagggacagcaaaggggtgc

SEQ ID NO: 74 - RNA, 22 nt, Homo sapiens
  source 1..22: mol_type = transcribed RNA
gttgggacaagaggacggtctt

SEQ ID NO: 75 - RNA, 23 nt, Homo sapiens
  source 1..23: mol_type = transcribed RNA
agcagcattgtacagggctatga

SEQ ID NO: 76 - RNA, 20 nt, Homo sapiens
  source 1..20: mol_type = transcribed RNA
aaaagctgggctgagaggcg

SEQ ID NO: 77 - RNA, 20 nt, Homo sapiens
  source 1..20: mol_type = transcribed RNA
ggggagctgtggaagcagta

SEQ ID NO: 78 - RNA, 22 nt, Homo sapiens
  source 1..22: mol_type = transcribed RNA
agctctgctgctcactggcagt

SEQ ID NO: 79 - RNA, 23 nt, Homo sapiens
  source 1..23: mol_type = transcribed RNA
atgcggacctgggttagcggagt

SEQ ID NO: 80 - RNA, 23 nt, Homo sapiens
  source 1..23: mol_type = transcribed RNA
gtgagtgggagccccagtgtgtg

SEQ ID NO: 81 - RNA, 23 nt, Homo sapiens
  source 1..23: mol_type = transcribed RNA
tggggacgtagctggccagacag

SEQ ID NO: 82 - RNA, 22 nt, Homo sapiens
  source 1..22: mol_type = transcribed RNA
agccaggctctgaagggaaagt

SEQ ID NO: 83 - RNA, 21 nt, Homo sapiens
  source 1..21: mol_type = transcribed RNA
agtggcaaagtctttccatat

SEQ ID NO: 84 - RNA, 22 nt, Homo sapiens
  source 1..22: mol_type = transcribed RNA
aggccatcagcagtccaatgaa

SEQ ID NO: 85 - RNA, 22 nt, Homo sapiens
  source 1..22: mol_type = transcribed RNA
actgggtaggtggggctccagg

SEQ ID NO: 86 - RNA, 22 nt, Homo sapiens
  source 1..22: mol_type = transcribed RNA
ctcggcgcggggcgcgggctcc

SEQ ID NO: 87 - RNA, 22 nt, Homo sapiens
  source 1..22: mol_type = transcribed RNA
gctgggattacaggcatgagcc

SEQ ID NO: 88 - RNA, 20 nt, Homo sapiens
  source 1..20: mol_type = transcribed RNA
ggctccttggtctaggggta

SEQ ID NO: 89 - RNA, 23 nt, Homo sapiens
  source 1..23: mol_type = transcribed RNA
gtgagtgtggatcctggaggaat

SEQ ID NO: 90 - RNA, 22 nt, Homo sapiens
  source 1..22: mol_type = transcribed RNA
aagctgccagttgaagaactgt

SEQ ID NO: 91 - RNA, 22 nt, Homo sapiens
  source 1..22: mol_type = transcribed RNA
gtggaccaggatggcaagggct

SEQ ID NO: 92 - RNA, 18 nt, Homo sapiens
  source 1..18: mol_type = transcribed RNA
aaagctgggttgagaagg

SEQ ID NO: 93 - RNA, 22 nt, Homo sapiens
  source 1..22: mol_type = transcribed RNA
tgagcccctgtgccgcccccag

SEQ ID NO: 94 - RNA, 21 nt, Homo sapiens
  source 1..21: mol_type = transcribed RNA
tgagggacccaggacaggaga

SEQ ID NO: 95 - RNA, 17 nt, Homo sapiens
  source 1..17: mol_type = transcribed RNA
ggatggaggaggggtct

SEQ ID NO: 96 - RNA, 22 nt, Homo sapiens
  source 1..22: mol_type = transcribed RNA
aggggactggttaatagaacta

SEQ ID NO: 97 - RNA, 19 nt, Homo sapiens
  source 1..19: mol_type = transcribed RNA
aagtgtgcagggcactggt

SEQ ID NO: 98 - RNA, 22 nt, Homo sapiens
  source 1..22: mol_type = transcribed RNA
cagttgggtctaggggtcagga

SEQ ID NO: 99 - RNA, 23 nt, Homo sapiens
  source 1..23: mol_type = transcribed RNA
agcagcattgtacagggctatca

SEQ ID NO: 100 - RNA, 21 nt, Homo sapiens
  source 1..21: mol_type = transcribed RNA
aggaggcagcgctctcaggac

SEQ ID NO: 101 - RNA, 22 nt, Homo sapiens
  source 1..22: mol_type = transcribed RNA
ggctttctagtctcagctctcc

SEQ ID NO: 102 - RNA, 22 nt, Homo sapiens
  source 1..22: mol_type = transcribed RNA
agggggcgcagtcactgacgtg

SEQ ID NO: 103 - RNA, 22 nt, Homo sapiens
  source 1..22: mol_type = transcribed RNA
agcagaggcagagaggctcagg

SEQ ID NO: 104 - RNA, 23 nt, Homo sapiens
  source 1..23: mol_type = transcribed RNA
ccagagcagcctgcggtaacagt

SEQ ID NO: 105 - RNA, 20 nt, Homo sapiens
  source 1..20: mol_type = transcribed RNA
gccggggctttgggtgaggg

SEQ ID NO: 106 - RNA, 23 nt, Homo sapiens
  source 1..23: mol_type = transcribed RNA
cagggagaaggtggaagtgcaga

SEQ ID NO: 107 - RNA, 21 nt, Homo sapiens
  source 1..21: mol_type = transcribed RNA
tcgaggagctcacagtctagt

SEQ ID NO: 108 - RNA, 22 nt, Homo sapiens
  source 1..22: mol_type = transcribed RNA
tctggggatgaggacagtgtgt

SEQ ID NO: 109 - RNA, 21 nt, Homo sapiens
  source 1..21: mol_type = transcribed RNA
aggtgggtatggaggagccct

SEQ ID NO: 110 - RNA, 17 nt, Homo sapiens
  source 1..17: mol_type = transcribed RNA
cgcgccgggcccgggtt

SEQ ID NO: 111 - RNA, 21 nt, Homo sapiens
  source 1..21: mol_type = transcribed RNA
tggggctagtgatgcaggacg

SEQ ID NO: 112 - RNA, 24 nt, Homo sapiens
  source 1..24: mol_type = transcribed RNA
ccccgggaacgtcgagactggagc

SEQ ID NO: 113 - RNA, 22 nt, Homo sapiens
  source 1..22: mol_type = transcribed RNA
caggcacgggagctcaggtgag

SEQ ID NO: 114 - RNA, 22 nt, Homo sapiens
  source 1..22: mol_type = transcribed RNA
tggctcagttcagcaggaacag

SEQ ID NO: 115 - RNA, 18 nt, Homo sapiens
  source 1..18: mol_type = transcribed RNA
atcccacctctgccacca

SEQ ID NO: 116 - RNA, 23 nt, Homo sapiens
  source 1..23: mol_type = transcribed RNA
gaacgcctgttcttgccaggtgg

SEQ ID NO: 117 - RNA, 21 nt, Homo sapiens
  source 1..21: mol_type = transcribed RNA
ttggggtggtcggccctggag

SEQ ID NO: 118 - RNA, 17 nt, Homo sapiens
  source 1..17: mol_type = transcribed RNA
gggagtctacagcaggg

SEQ ID NO: 119 - RNA, 22 nt, Homo sapiens
  source 1..22: mol_type = transcribed RNA
ggctggagcgagtgcagtggtg

SEQ ID NO: 120 - RNA, 20 nt, Homo sapiens
  source 1..20: mol_type = transcribed RNA
atatgcctggctagctcctc

SEQ ID NO: 121 - RNA, 19 nt, Homo sapiens
  source 1..19: mol_type = transcribed RNA
aaaagctgggttgagagga

SEQ ID NO: 122 - RNA, 22 nt, Homo sapiens
  source 1..22: mol_type = transcribed RNA
agttgcctttttgttcccatgc

SEQ ID NO: 123 - RNA, 21 nt, Homo sapiens
  source 1..21: mol_type = transcribed RNA
agggccccccctcaatcctgt

SEQ ID NO: 124 - RNA, 21 nt, Homo sapiens
  source 1..21: mol_type = transcribed RNA
atgcctcccccggccccgcag

SEQ ID NO: 125 - RNA, 23 nt, Homo sapiens
  source 1..23: mol_type = transcribed RNA
gcggtgatcccgatggtgtgagc

SEQ ID NO: 126 - RNA, 22 nt, Homo sapiens
  source 1..22: mol_type = transcribed RNA
actcaaaatgggggcgctttcc

SEQ ID NO: 127 - RNA, 22 nt, Homo sapiens
  source 1..22: mol_type = transcribed RNA
tgggatccagacagtgggagaa

SEQ ID NO: 128 - RNA, 21 nt, Homo sapiens
  source 1..21: mol_type = transcribed RNA
agcatgacagaggagaggtgg

SEQ ID NO: 129 - RNA, 20 nt, Homo sapiens
  source 1..20: mol_type = transcribed RNA
gctcggactgagcaggtggg

SEQ ID NO: 130 - RNA, 17 nt, Homo sapiens
  source 1..17: mol_type = transcribed RNA
atgtgggctcaggctca

SEQ ID NO: 131 - RNA, 22 nt, Homo sapiens
  source 1..22: mol_type = transcribed RNA
ggaggggtcccgcactgggagg

SEQ ID NO: 132 - RNA, 23 nt, Homo sapiens
  source 1..23: mol_type = transcribed RNA
ttagggagtagaagggtggggag

SEQ ID NO: 133 - RNA, 18 nt, Homo sapiens
  source 1..18: mol_type = transcribed RNA
aggctggagtgagcggag

SEQ ID NO: 134 - RNA, 22 nt, Homo sapiens
  source 1..22: mol_type = transcribed RNA
tcctgtactgagctgccccgag

SEQ ID NO: 135 - RNA, 17 nt, Homo sapiens
  source 1..17: mol_type = transcribed RNA
ggtgggggctgttgttt

SEQ ID NO: 136 - RNA, 23 nt, Homo sapiens
  source 1..23: mol_type = transcribed RNA
ccagggggatgggcgagcttggg

SEQ ID NO: 137 - RNA, 22 nt, Homo sapiens
  source 1..22: mol_type = transcribed RNA
aaggcccgggctttcctcccag

SEQ ID NO: 138 - RNA, 18 nt, Homo sapiens
  source 1..18: mol_type = transcribed RNA
tcgaggagctcacagtct

SEQ ID NO: 139 - RNA, 20 nt, Homo sapiens
  source 1..20: mol_type = transcribed RNA
gaatcggaaaggaggcgccg

SEQ ID NO: 140 - RNA, 21 nt, Homo sapiens
  source 1..21: mol_type = transcribed RNA
atacatgtcagattgtatgcc

SEQ ID NO: 141 - RNA, 22 nt, Homo sapiens
  source 1..22: mol_type = transcribed RNA
agacacatttggagagggaccc

SEQ ID NO: 142 - RNA, 25 nt, Homo sapiens
  source 1..25: mol_type = transcribed RNA
cctccaaagggaagcgctttctgtt

SEQ ID NO: 143 - RNA, 22 nt, Homo sapiens
  source 1..22: mol_type = transcribed RNA
gtgactgataccttggaggcat

SEQ ID NO: 144 - RNA, 17 nt, Homo sapiens
  source 1..17: mol_type = transcribed RNA
atggagaaggcttctga

SEQ ID NO: 145 - RNA, 22 nt, Homo sapiens
  source 1..22: mol_type = transcribed RNA
ttggggaaacggccgctgagtg

SEQ ID NO: 146 - RNA, 21 nt, Homo sapiens
  source 1..21: mol_type = transcribed RNA
taccacagggtagaaccacgg

SEQ ID NO: 147 - RNA, 22 nt, Homo sapiens
  source 1..22: mol_type = transcribed RNA
gagcaggcgaggctgggctgaa

SEQ ID NO: 148 - RNA, 24 nt, Homo sapiens
  source 1..24: mol_type = transcribed RNA
agctgagctccatggacgtgcagt

SEQ ID NO: 149 - RNA, 23 nt, Homo sapiens
  source 1..23: mol_type = transcribed RNA
tctctggagggaagcactttctg

SEQ ID NO: 150 - RNA, 21 nt, Homo sapiens
  source 1..21: mol_type = transcribed RNA
cgggccggaggtcaagggcgt

SEQ ID NO: 151 - RNA, 22 nt, Homo sapiens
  source 1..22: mol_type = transcribed RNA
ggtggttgaggctgcagtaagt

SEQ ID NO: 152 - RNA, 22 nt, Homo sapiens
  source 1..22: mol_type = transcribed RNA
tgagaccaggactggatgcacc

SEQ ID NO: 153 - RNA, 22 nt, Homo sapiens
  source 1..22: mol_type = transcribed RNA
gagcttggatgagctgggctga

SEQ ID NO: 154 - RNA, 23 nt, Homo sapiens
  source 1..23: mol_type = transcribed RNA
gattgagactagtagggctaggc

SEQ ID NO: 155 - RNA, 20 nt, Homo sapiens
  source 1..20: mol_type = transcribed RNA
ggtagtgagttatcagctac

SEQ ID NO: 156 - RNA, 24 nt, Homo sapiens
  source 1..24: mol_type = transcribed RNA
cgggtgggagcagatcttattgag

SEQ ID NO: 157 - RNA, 23 nt, Homo sapiens
  source 1..23: mol_type = transcribed RNA
cggggccatggagcagcctgtgt

SEQ ID NO: 158 - RNA, 22 nt, Homo sapiens
  source 1..22: mol_type = transcribed RNA
atcaaggatcttaaactttgcc

SEQ ID NO: 159 - RNA, 16 nt, Homo sapiens
  source 1..16: mol_type = transcribed RNA
aggaaacagggaccca

SEQ ID NO: 160 - RNA, 17 nt, Homo sapiens
  source 1..17: mol_type = transcribed RNA
ccccggggagcccggcg

SEQ ID NO: 161 - RNA, 21 nt, Homo sapiens
  source 1..21: mol_type = transcribed RNA
gtgccagctgcagtgggggag

SEQ ID NO: 162 - RNA, 22 nt, Homo sapiens
  source 1..22: mol_type = transcribed RNA
tagcagcacgtaaatattggcg

SEQ ID NO: 163 - RNA, 21 nt, Homo sapiens
  source 1..21: mol_type = transcribed RNA
ttggagggtgtggaagacatc

SEQ ID NO: 164 - RNA, 22 nt, Homo sapiens
  source 1..22: mol_type = transcribed RNA
tcgggcctggggttgggggagc

SEQ ID NO: 165 - RNA, 22 nt, Homo sapiens
  source 1..22: mol_type = transcribed RNA
cagggcaggaagaagtggacaa

SEQ ID NO: 166 - RNA, 23 nt, Homo sapiens
  source 1..23: mol_type = transcribed RNA
agctacattgtctgctgggtttc

SEQ ID NO: 167 - RNA, 23 nt, Homo sapiens
  source 1..23: mol_type = transcribed RNA
gccccggcgcgggcgggttctgg

SEQ ID NO: 168 - RNA, 26 nt, Homo sapiens
  source 1..26: mol_type = transcribed RNA
cccagggcttggagtggggcaaggtt

SEQ ID NO: 169 - RNA, 22 nt, Homo sapiens
  source 1..22: mol_type = transcribed RNA
ggggaactgtagatgaaaaggc

SEQ ID NO: 170 - RNA, 24 nt, Homo sapiens
  source 1..24: mol_type = transcribed RNA
tgggctaagggagatgattgggta

SEQ ID NO: 171 - RNA, 21 nt, Homo sapiens
  source 1..21: mol_type = transcribed RNA
tcacctggctggcccgcccag

SEQ ID NO: 172 - RNA, 25 nt, Homo sapiens
  source 1..25: mol_type = transcribed RNA
atggggtgagatggggaggagcagc

SEQ ID NO: 173 - RNA, 23 nt, Homo sapiens
  source 1..23: mol_type = transcribed RNA
tcaataggaaagaggtgggacct

SEQ ID NO: 174 - RNA, 22 nt, Homo sapiens
  source 1..22: mol_type = transcribed RNA
cgcgcctgcaggaactggtaga

SEQ ID NO: 175 - RNA, 22 nt, Homo sapiens
  source 1..22: mol_type = transcribed RNA
caagggaccaagcattcattat

SEQ ID NO: 176 - RNA, 22 nt, Homo sapiens
  source 1..22: mol_type = transcribed RNA
gaaatcaagcgtgggtgagacc

SEQ ID NO: 177 - RNA, 18 nt, Homo sapiens
  source 1..18: mol_type = transcribed RNA
gaggctgaaggaagatgg

SEQ ID NO: 178 - RNA, 20 nt, Homo sapiens
  source 1..20: mol_type = transcribed RNA
gtgggttggggcgggctctg

SEQ ID NO: 179 - RNA, 22 nt, Homo sapiens
  source 1..22: mol_type = transcribed RNA
tggagtgtgacaatggtgtttg

SEQ ID NO: 180 - RNA, 22 nt, Homo sapiens
  source 1..22: mol_type = transcribed RNA
aaagatctggaagtgggagaca

SEQ ID NO: 181 - RNA, 25 nt, Homo sapiens
  source 1..25: mol_type = transcribed RNA
aggggggcactgcgcaagcaaagcc

SEQ ID NO: 182 - RNA, 22 nt, Homo sapiens
  source 1..22: mol_type = transcribed RNA
ctggggagatcctcgaggttgg

SEQ ID NO: 183 - RNA, 18 nt, Homo sapiens
  source 1..18: mol_type = transcribed RNA
acaggcaggattggggaa

SEQ ID NO: 184 - RNA, 18 nt, Homo sapiens
  source 1..18: mol_type = transcribed RNA
cggctggaggtgtgagga

SEQ ID NO: 185 - RNA, 22 nt, Homo sapiens
  source 1..22: mol_type = transcribed RNA
gaaggcagcagtgctcccctgt

SEQ ID NO: 186 - RNA, 22 nt, Homo sapiens
  source 1..22: mol_type = transcribed RNA
cagtgcaatgttaaaagggcat

SEQ ID NO: 187 - RNA, 21 nt, Homo sapiens
  source 1..21: mol_type = transcribed RNA
tggatgtggaaggagttatct

SEQ ID NO: 188 - RNA, 23 nt, Homo sapiens
  source 1..23: mol_type = transcribed RNA
ccccggtgttggggcgcgtctgc

SEQ ID NO: 189 - RNA, 21 nt, Homo sapiens
  source 1..21: mol_type = transcribed RNA
gtaccttctggttcagctagt

SEQ ID NO: 190 - RNA, 21 nt, Homo sapiens
  source 1..21: mol_type = transcribed RNA
attggactgctgatggcccgt

SEQ ID NO: 191 - RNA, 20 nt, Homo sapiens
  source 1..20: mol_type = transcribed RNA
gtgagtcagggtggggctgg

SEQ ID NO: 192 - RNA, 17 nt, Homo sapiens
  source 1..17: mol_type = transcribed RNA
tgaggtagtagtttctt

SEQ ID NO: 193 - RNA, 18 nt, Homo sapiens
  source 1..18: mol_type = transcribed RNA
ctcgagttggaagaggcg

SEQ ID NO: 194 - RNA, 22 nt, Homo sapiens
  source 1..22: mol_type = transcribed RNA
tgaggtagtaggttgtatagtt

SEQ ID NO: 195 - RNA, 22 nt, Homo sapiens
  source 1..22: mol_type = transcribed RNA
ctctagagggaagcgctttctg

SEQ ID NO: 196 - RNA, 18 nt, Homo sapiens
  source 1..18: mol_type = transcribed RNA
tgagatgacactgtagct

SEQ ID NO: 197 - RNA, 19 nt, Homo sapiens
  source 1..19: mol_type = transcribed RNA
cggcgcgaccggcccgggg

SEQ ID NO: 198 - RNA, 23 nt, Homo sapiens
  source 1..23: mol_type = transcribed RNA
gagaggaacatgggctcaggaca

SEQ ID NO: 199 - RNA, 22 nt, Homo sapiens
  source 1..22: mol_type = transcribed RNA
ctggggttctgagacagacagt

SEQ ID NO: 200 - RNA, 21 nt, Homo sapiens
  source 1..21: mol_type = transcribed RNA
tagtggtcagagggcttatga

SEQ ID NO: 201 - RNA, 20 nt, Homo sapiens
  source 1..20: mol_type = transcribed RNA
aagtcctgcttctgttgcag

SEQ ID NO: 202 - RNA, 22 nt, Homo sapiens
  source 1..22: mol_type = transcribed RNA
tcaggtgtggaaactgaggcag

SEQ ID NO: 203 - RNA, 22 nt, Homo sapiens
  source 1..22: mol_type = transcribed RNA
ccctgtgcccggcccacttctg

SEQ ID NO: 204 - RNA, 22 nt, Homo sapiens
  source 1..22: mol_type = transcribed RNA
ggggggtgtggagccagggggc

SEQ ID NO: 205 - RNA, 22 nt, Homo sapiens
  source 1..22: mol_type = transcribed RNA
ctacaaagggaagcactttctc

SEQ ID NO: 206 - RNA, 20 nt, Homo sapiens
  source 1..20: mol_type = transcribed RNA
aggcgcacccgaccacatgc

SEQ ID NO: 207 - RNA, 22 nt, Homo sapiens
  source 1..22: mol_type = transcribed RNA
tatacctcagttttatcaggtg

SEQ ID NO: 208 - RNA, 20 nt, Homo sapiens
  source 1..20: mol_type = transcribed RNA
tctcgcatcaggaggcaagg

SEQ ID NO: 209 - RNA, 18 nt, Homo sapiens
  source 1..18: mol_type = transcribed RNA
aggagaagtaaagtagaa

SEQ ID NO: 210 - RNA, 23 nt, Homo sapiens
  source 1..23: mol_type = transcribed RNA
gtgcttcatcgtaattaacctta

SEQ ID NO: 211 - RNA, 19 nt, Homo sapiens
  source 1..19: mol_type = transcribed RNA
gagccagttggacaggagc

SEQ ID NO: 212 - RNA, 22 nt, Homo sapiens
  source 1..22: mol_type = transcribed RNA
cagagtgacaagctggttaaag

SEQ ID NO: 213 - RNA, 23 nt, Homo sapiens
  source 1..23: mol_type = transcribed RNA
ctgggcccgcggcgggcgtgggg

SEQ ID NO: 214 - RNA, 23 nt, Homo sapiens
  source 1..23: mol_type = transcribed RNA
ctgggggacgcgtgagcgcgagc

SEQ ID NO: 215 - RNA, 22 nt, Homo sapiens
  source 1..22: mol_type = transcribed RNA
tgacctgggactcggacagctg

SEQ ID NO: 216 - RNA, 25 nt, Homo sapiens
  source 1..25: mol_type = transcribed RNA
ggcggagggaagtaggtccgttggt

SEQ ID NO: 217 - RNA, 23 nt, Homo sapiens
  source 1..23: mol_type = transcribed RNA
tgtagagcagggagcaggaagct

SEQ ID NO: 218 - RNA, 22 nt, Homo sapiens
  source 1..22: mol_type = transcribed RNA
tggcccaacctattcagttagt

SEQ ID NO: 219 - RNA, 22 nt, Homo sapiens
  source 1..22: mol_type = transcribed RNA
caggtagatatttgataggcat

SEQ ID NO: 220 - RNA, 21 nt, Homo sapiens
  source 1..21: mol_type = transcribed RNA
ctgtgggctcagcgcgtgggg

SEQ ID NO: 221 - RNA, 22 nt, Homo sapiens
  source 1..22: mol_type = transcribed RNA
gtccacttctgcctgccctgcc

SEQ ID NO: 222 - RNA, 22 nt, Homo sapiens
  source 1..22: mol_type = transcribed RNA
agaaagggtggcaatacctctt

SEQ ID NO: 223 - RNA, 22 nt, Homo sapiens
  source 1..22: mol_type = transcribed RNA
tcaatcacttggtaattgctgt

SEQ ID NO: 224 - RNA, 20 nt, Homo sapiens
  source 1..20: mol_type = transcribed RNA
cagggggactgggggtgagc

SEQ ID NO: 225 - RNA, 22 nt, Homo sapiens
  source 1..22: mol_type = transcribed RNA
taggagggaatagtaaaagcag

SEQ ID NO: 226 - RNA, 22 nt, Homo sapiens
  source 1..22: mol_type = transcribed RNA
atattattagccacttctggat

SEQ ID NO: 227 - RNA, 22 nt, Homo sapiens
  source 1..22: mol_type = transcribed RNA
gcccgcgtgtggagccaggtgt

SEQ ID NO: 228 - RNA, 22 nt, Homo sapiens
  source 1..22: mol_type = transcribed RNA
aggagctagccaggcatatgca

SEQ ID NO: 229 - RNA, 22 nt, Homo sapiens
  source 1..22: mol_type = transcribed RNA
tagcagcacatcatggtttaca

SEQ ID NO: 230 - RNA, 22 nt, Homo sapiens
  source 1..22: mol_type = transcribed RNA
ctctagagggaagcactttctg

SEQ ID NO: 231 - RNA, 22 nt, Homo sapiens
  source 1..22: mol_type = transcribed RNA
taaggggtgtatggcagatgca

SEQ ID NO: 232 - RNA, 22 nt, Homo sapiens
  source 1..22: mol_type = transcribed RNA
agcccctgcccaccgcacactg

SEQ ID NO: 233 - RNA, 22 nt, Homo sapiens
  source 1..22: mol_type = transcribed RNA
aggtggatgcaatgtgacctca

SEQ ID NO: 234 - RNA, 23 nt, Homo sapiens
  source 1..23: mol_type = transcribed RNA
aggcctctgtgacgtcacggtgt

SEQ ID NO: 235 - RNA, 22 nt, Homo sapiens
  source 1..22: mol_type = transcribed RNA
tcacaaggtattgactggcgta

SEQ ID NO: 236 - RNA, 22 nt, Homo sapiens
  source 1..22: mol_type = transcribed RNA
gtatacacctgatatgtgtatg

SEQ ID NO: 237 - RNA, 23 nt, Homo sapiens
  source 1..23: mol_type = transcribed RNA
tggggggacagatggagaggaca

SEQ ID NO: 238 - RNA, 22 nt, Homo sapiens
  source 1..22: mol_type = transcribed RNA
atatgtatatgtgactgctact

SEQ ID NO: 239 - RNA, 22 nt, Homo sapiens
  source 1..22: mol_type = transcribed RNA
ttagcttaaggagtaccagatc

SEQ ID NO: 240 - RNA, 21 nt, Homo sapiens
  source 1..21: mol_type = transcribed RNA
aaggtttggatagatgcaata

SEQ ID NO: 241 - RNA, 22 nt, Homo sapiens
  source 1..22: mol_type = transcribed RNA
tgaagccagctctggtctgggc

SEQ ID NO: 242 - RNA, 22 nt, Homo sapiens
  source 1..22: mol_type = transcribed RNA
aggcagcggggtgtagtggata

SEQ ID NO: 243 - RNA, 23 nt, Homo sapiens
  source 1..23: mol_type = transcribed RNA
gggggccgatacactgtacgaga

SEQ ID NO: 244 - RNA, 22 nt, Homo sapiens
  source 1..22: mol_type = transcribed RNA
ttcaagtaatccaggataggct

SEQ ID NO: 245 - RNA, 23 nt, Homo sapiens
  source 1..23: mol_type = transcribed RNA
tagcaatacagtacaaatatagt

SEQ ID NO: 246 - RNA, 21 nt, Homo sapiens
  source 1..21: mol_type = transcribed RNA
tgggagctaagctatgggtat

SEQ ID NO: 247 - RNA, 22 nt, Homo sapiens
  source 1..22: mol_type = transcribed RNA
atacctcatctagaatgctgta

SEQ ID NO: 248 - RNA, 22 nt, Homo sapiens
  source 1..22: mol_type = transcribed RNA
tcatttatctgttgggaagcta

SEQ ID NO: 249 - RNA, 22 nt, Homo sapiens
  source 1..22: mol_type = transcribed RNA
gccctgaacgaggggtctggag

SEQ ID NO: 250 - RNA, 20 nt, Homo sapiens
  source 1..20: mol_type = transcribed RNA
tctcttcatctaccccccag

SEQ ID NO: 251 - RNA, 21 nt, Homo sapiens
  source 1..21: mol_type = transcribed RNA
atccagttctctgagggggct

SEQ ID NO: 252 - RNA, 22 nt, Homo sapiens
  source 1..22: mol_type = transcribed RNA
tcacaaatctataatatgcagg

SEQ ID NO: 253 - RNA, 21 nt, Homo sapiens
  source 1..21: mol_type = transcribed RNA
ggtggggcaatgggatcaggt

SEQ ID NO: 254 - RNA, 22 nt, Homo sapiens
  source 1..22: mol_type = transcribed RNA
aacccgtagatccgatcttgtg

SEQ ID NO: 255 - RNA, 21 nt, Homo sapiens
  source 1..21: mol_type = transcribed RNA
tggctgttggagggggcaggc

SEQ ID NO: 256 - RNA, 22 nt, Homo sapiens
  source 1..22: mol_type = transcribed RNA
tcctttgcctattctatttaag

SEQ ID NO: 257 - RNA, 22 nt, Homo sapiens
  source 1..22: mol_type = transcribed RNA
aagttctgttatacactcaggc

SEQ ID NO: 258 - RNA, 22 nt, Homo sapiens
  source 1..22: mol_type = transcribed RNA
tgtgatatcatggttcctggga

SEQ ID NO: 259 - RNA, 22 nt, Homo sapiens
  source 1..22: mol_type = transcribed RNA
cgggactgtagagggcatgagc

SEQ ID NO: 260 - RNA, 22 nt, Homo sapiens
  source 1..22: mol_type = transcribed RNA
acgcccttcccccccttcttca

SEQ ID NO: 261 - RNA, 20 nt, Homo sapiens
  source 1..20: mol_type = transcribed RNA
tcacacctgcctcgcccccc

SEQ ID NO: 262 - RNA, 22 nt, Homo sapiens
  source 1..22: mol_type = transcribed RNA
tctggtatgtagtaggtaataa

SEQ ID NO: 263 - RNA, 22 nt, Homo sapiens
  source 1..22: mol_type = transcribed RNA
aaggtattgttcagacttatga

SEQ ID NO: 264 - RNA, 22 nt, Homo sapiens
  source 1..22: mol_type = transcribed RNA
tttaacatgggggtacctgctg

SEQ ID NO: 265 - RNA, 22 nt, Homo sapiens
  source 1..22: mol_type = transcribed RNA
tgggtctttgcgggcgagatga

SEQ ID NO: 266 - RNA, 21 nt, Homo sapiens
  source 1..21: mol_type = transcribed RNA
tgtggtagatatatgcacgat

SEQ ID NO: 267 - RNA, 22 nt, Homo sapiens
  source 1..22: mol_type = transcribed RNA
gggagccaggaagtattgatgt

SEQ ID NO: 268 - RNA, 22 nt, Homo sapiens
  source 1..22: mol_type = transcribed RNA
aagctttttgctcgaattatgt

SEQ ID NO: 269 - RNA, 22 nt, Homo sapiens
  source 1..22: mol_type = transcribed RNA
tgaggtagtaggttgtgtggtt

SEQ ID NO: 270 - RNA, 21 nt, Homo sapiens
  source 1..21: mol_type = transcribed RNA
tgaggtgaccgcagatgggaa

SEQ ID NO: 271 - RNA, 22 nt, Homo sapiens
  source 1..22: mol_type = transcribed RNA
gagcaatgtaggtagactgttt

SEQ ID NO: 272 - RNA, 20 nt, Homo sapiens
  source 1..20: mol_type = transcribed RNA
agcacactgagcgagcggac

SEQ ID NO: 273 - RNA, 22 nt, Homo sapiens
  source 1..22: mol_type = transcribed RNA
tgctggatcagtggttcgagtc

SEQ ID NO: 274 - RNA, 22 nt, Homo sapiens
  source 1..22: mol_type = transcribed RNA
aaacctgtgttgttcaagagtc

SEQ ID NO: 275 - RNA, 23 nt, Homo sapiens
  source 1..23: mol_type = transcribed RNA
gtccagttttcccaggaatccct

SEQ ID NO: 276 - RNA, 22 nt, Homo sapiens
  source 1..22: mol_type = transcribed RNA
tgtgggtgggaaggactggatt

SEQ ID NO: 277 - RNA, 22 nt, Homo sapiens
  source 1..22: mol_type = transcribed RNA
cattgcacttgtctcggtctga

SEQ ID NO: 278 - RNA, 22 nt, Homo sapiens
  source 1..22: mol_type = transcribed RNA
cctctaaagggaagcgctttct

SEQ ID NO: 279 - RNA, 22 nt, Homo sapiens
  source 1..22: mol_type = transcribed RNA
aactagtaatgttggattaggg

SEQ ID NO: 280 - RNA, 22 nt, Homo sapiens
  source 1..22: mol_type = transcribed RNA
ttggggtggagggccaaggagc

SEQ ID NO: 281 - RNA, 23 nt, Homo sapiens
  source 1..23: mol_type = transcribed RNA
agaattgcgtttggacaatcagt

SEQ ID NO: 282 - RNA, 21 nt, Homo sapiens
  source 1..21: mol_type = transcribed RNA
cctgcagagaggaagcccttc

SEQ ID NO: 283 - RNA, 22 nt, Homo sapiens
  source 1..22: mol_type = transcribed RNA
tgtgggttctgggttggggtga

SEQ ID NO: 284 - RNA, 22 nt, Homo sapiens
  source 1..22: mol_type = transcribed RNA
tgtcagtttgtcaaatacccca

SEQ ID NO: 285 - RNA, 21 nt, Homo sapiens
  source 1..21: mol_type = transcribed RNA
ctggggggaggagaccctgct

SEQ ID NO: 286 - RNA, 22 nt, Homo sapiens
  source 1..22: mol_type = transcribed RNA
caaaaaccatagttacttttgc

SEQ ID NO: 287 - RNA, 22 nt, Homo sapiens
  source 1..22: mol_type = transcribed RNA
tgaggtagtaggttgtatggtt

SEQ ID NO: 288 - RNA, 22 nt, Homo sapiens
  source 1..22: mol_type = transcribed RNA
agaaggcactatgagatttaga

SEQ ID NO: 289 - RNA, 23 nt, Homo sapiens
  source 1..23: mol_type = transcribed RNA
acggagacgacaagactgtgctg

SEQ ID NO: 290 - RNA, 20 nt, Homo sapiens
  source 1..20: mol_type = transcribed RNA
agacactatacgagtcatat

SEQ ID NO: 291 - RNA, 22 nt, Homo sapiens
  source 1..22: mol_type = transcribed RNA
tcttcttcctttgcagagttga

SEQ ID NO: 292 - RNA, 21 nt, Homo sapiens
  source 1..21: mol_type = transcribed RNA
attgacacttctgtgagtaga

SEQ ID NO: 293 - RNA, 20 nt, Homo sapiens
  source 1..20: mol_type = transcribed RNA
atatgggtttactagttggt

SEQ ID NO: 294 - RNA, 21 nt, Homo sapiens
  source 1..21: mol_type = transcribed RNA
agaagtggctaataatattga

SEQ ID NO: 295 - RNA, 20 nt, Homo sapiens
  source 1..20: mol_type = transcribed RNA
tcttttctttgagactcact

SEQ ID NO: 296 - RNA, 22 nt, Homo sapiens
  source 1..22: mol_type = transcribed RNA
atggcatcgtcccctggtggct

SEQ ID NO: 297 - RNA, 22 nt, Homo sapiens
  source 1..22: mol_type = transcribed RNA
caaggccaaaggaagagaacag

SEQ ID NO: 298 - RNA, 20 nt, Homo sapiens
  source 1..20: mol_type = transcribed RNA
cagggaaccagttggggctt

SEQ ID NO: 299 - RNA, 23 nt, Homo sapiens
  source 1..23: mol_type = transcribed RNA
tttcaagccagggggcgtttttc

SEQ ID NO: 300 - RNA, 22 nt, Homo sapiens
  source 1..22: mol_type = transcribed RNA
ttctcaagagggaggcaatcat

SEQ ID NO: 301 - RNA, 22 nt, Homo sapiens
  source 1..22: mol_type = transcribed RNA
aaaatggttccctttagagtgt

SEQ ID NO: 302 - RNA, 22 nt, Homo sapiens
  source 1..22: mol_type = transcribed RNA
taccattagaagagctggaaga

SEQ ID NO: 303 - RNA, 21 nt, Homo sapiens
  source 1..21: mol_type = transcribed RNA
aggtagaatgaggcctgacat

SEQ ID NO: 304 - RNA, 22 nt, Homo sapiens
  source 1..22: mol_type = transcribed RNA
tagggatgggaggccaggatga

SEQ ID NO: 305 - RNA, 20 nt, Homo sapiens
  source 1..20: mol_type = transcribed RNA
ctacaaagggaagccctttc

SEQ ID NO: 306 - RNA, 23 nt, Homo sapiens
  source 1..23: mol_type = transcribed RNA
agggacttttgggggcagatgtg

SEQ ID NO: 307 - RNA, 23 nt, Homo sapiens
  source 1..23: mol_type = transcribed RNA
aagaagagactgagtcatcgaat

SEQ ID NO: 308 - RNA, 22 nt, Homo sapiens
  source 1..22: mol_type = transcribed RNA
aacccagtgggctatggaaatg

SEQ ID NO: 309 - RNA, 22 nt, Homo sapiens
  source 1..22: mol_type = transcribed RNA
agaggttatccgtgttatgttc

SEQ ID NO: 310 - RNA, 23 nt, Homo sapiens
  source 1..23: mol_type = transcribed RNA
caggcagaagtggggctgacagg

SEQ ID NO: 311 - RNA, 22 nt, Homo sapiens
  source 1..22: mol_type = transcribed RNA
ttctggataacatgctgaagct

SEQ ID NO: 312 - RNA, 20 nt, Homo sapiens
  source 1..20: mol_type = transcribed RNA
aaaagtgattgcagtgtttg

SEQ ID NO: 313 - RNA, 24 nt, Homo sapiens
  source 1..24: mol_type = transcribed RNA
tctgggcacaggcggatggacagg

SEQ ID NO: 314 - RNA, 20 nt, Homo sapiens
  source 1..20: mol_type = transcribed RNA
gagcttggtctgtagcggtt

SEQ ID NO: 315 - RNA, 22 nt, Homo sapiens
  source 1..22: mol_type = transcribed RNA
ttacacacaactgaggatcata

SEQ ID NO: 316 - RNA, 23 nt, Homo sapiens
  source 1..23: mol_type = transcribed RNA
cagggaggcgctcactctctgct

SEQ ID NO: 317 - RNA, 22 nt, Homo sapiens
  source 1..22: mol_type = transcribed RNA
gagcttattcataaaagtgcag

SEQ ID NO: 318 - RNA, 22 nt, Homo sapiens
  source 1..22: mol_type = transcribed RNA
gaaaacgacaatgacttttgca

SEQ ID NO: 319 - RNA, 22 nt, Homo sapiens
  source 1..22: mol_type = transcribed RNA
agaaggggtgaaatttaaacgt

SEQ ID NO: 320 - RNA, 22 nt, Homo sapiens
  source 1..22: mol_type = transcribed RNA
agcatacacctgtagtcctaga

SEQ ID NO: 321 - RNA, 21 nt, Homo sapiens
  source 1..21: mol_type = transcribed RNA
ggtggatattccttctatgtt

SEQ ID NO: 322 - RNA, 22 nt, Homo sapiens
  source 1..22: mol_type = transcribed RNA
tctggtaagagatttgggcata

SEQ ID NO: 323 - RNA, 23 nt, Homo sapiens
  source 1..23: mol_type = transcribed RNA
ctcttgagggaagcactttctgt

SEQ ID NO: 324 - RNA, 23 nt, Homo sapiens
  source 1..23: mol_type = transcribed RNA
tgagagtggaattcacagtattt

SEQ ID NO: 325 - RNA, 22 nt, Homo sapiens
  source 1..22: mol_type = transcribed RNA
actcggcgtggcgtcggtcgtg

SEQ ID NO: 326 - RNA, 22 nt, Homo sapiens
  source 1..22: mol_type = transcribed RNA
atctgtaagagaaagtaaatga

SEQ ID NO: 327 - RNA, 22 nt, Homo sapiens
  source 1..22: mol_type = transcribed RNA
ggggctgtgattgaccagcagg

SEQ ID NO: 328 - RNA, 23 nt, Homo sapiens
  source 1..23: mol_type = transcribed RNA
caaagtgctcatagtgcaggtag

SEQ ID NO: 329 - RNA, 21 nt, Homo sapiens
  source 1..21: mol_type = transcribed RNA
actgacaggagagcattttga

SEQ ID NO: 330 - RNA, 22 nt, Homo sapiens
  source 1..22: mol_type = transcribed RNA
caacctcgacgatctcctcagc

SEQ ID NO: 331 - RNA, 21 nt, Homo sapiens
  source 1..21: mol_type = transcribed RNA
gaagaataggagggactttgt

SEQ ID NO: 332 - RNA, 23 nt, Homo sapiens
  source 1..23: mol_type = transcribed RNA
tgagcaccacacaggccgggcgc

SEQ ID NO: 333 - RNA, 21 nt, Homo sapiens
  source 1..21: mol_type = transcribed RNA
agggtaagctgaacctctgat

SEQ ID NO: 334 - RNA, 21 nt, Homo sapiens
  source 1..21: mol_type = transcribed RNA
gcggtggggccggaggggcgt

SEQ ID NO: 335 - RNA, 21 nt, Homo sapiens
  source 1..21: mol_type = transcribed RNA
tggtagactatggaacgtagg

SEQ ID NO: 336 - RNA, 18 nt, Homo sapiens
  source 1..18: mol_type = transcribed RNA
aaaagtacttgcggattt

SEQ ID NO: 337 - RNA, 21 nt, Homo sapiens
  source 1..21: mol_type = transcribed RNA
agttaggattaggtcgtggaa

SEQ ID NO: 338 - RNA, 22 nt, Homo sapiens
  source 1..22: mol_type = transcribed RNA
ctggtttcacatggtggcttag

SEQ ID NO: 339 - RNA, 20 nt, Homo sapiens
  source 1..20: mol_type = transcribed RNA
agaggtatagggcatgggaa

SEQ ID NO: 340 - RNA, 21 nt, Homo sapiens
  source 1..21: mol_type = transcribed RNA
ccaggctctgcagtgggaact

SEQ ID NO: 341 - RNA, 21 nt, Homo sapiens
  source 1..21: mol_type = transcribed RNA
ttcacagtggctaagttctgc

SEQ ID NO: 342 - RNA, 23 nt, Homo sapiens
  source 1..23: mol_type = transcribed RNA
gagtggataggggagtgtgtgga

SEQ ID NO: 343 - RNA, 22 nt, Homo sapiens
  source 1..22: mol_type = transcribed RNA
tagcagcacataatggtttgtg

SEQ ID NO: 344 - RNA, 23 nt, Homo sapiens
  source 1..23: mol_type = transcribed RNA
caatggcacaaactcattcttga

SEQ ID NO: 345 - RNA, 22 nt, Homo sapiens
  source 1..22: mol_type = transcribed RNA
ttaggccatcatcccattatgc

SEQ ID NO: 346 - RNA, 22 nt, Homo sapiens
  source 1..22: mol_type = transcribed RNA
taaagaactcttaaaacccaat

SEQ ID NO: 347 - RNA, 22 nt, Homo sapiens
  source 1..22: mol_type = transcribed RNA
tgcaagacggatactgtcatct

SEQ ID NO: 348 - RNA, 21 nt, Homo sapiens
  source 1..21: mol_type = transcribed RNA
ttcgcgggcgaaggcaaagtc

SEQ ID NO: 349 - RNA, 21 nt, Homo sapiens
  source 1..21: mol_type = transcribed RNA
cgagcctcaagcaagggactt

SEQ ID NO: 350 - RNA, 22 nt, Homo sapiens
  source 1..22: mol_type = transcribed RNA
ccaaggaaggaggctggacatc

SEQ ID NO: 351 - RNA, 18 nt, Homo sapiens
  source 1..18: mol_type = transcribed RNA
cccagcaggacgggagcg

SEQ ID NO: 352 - RNA, 22 nt, Homo sapiens
  source 1..22: mol_type = transcribed RNA
ctattaaggacatttgtgattc

SEQ ID NO: 353 - RNA, 22 nt, Homo sapiens
  source 1..22: mol_type = transcribed RNA
tcgtaccgtgagtaataatgcg

SEQ ID NO: 354 - RNA, 22 nt, Homo sapiens
  source 1..22: mol_type = transcribed RNA
agctgtaattagtcagttttct

SEQ ID NO: 355 - RNA, 17 nt, Homo sapiens
  source 1..17: mol_type = transcribed RNA
tggtagagctgaggaca

SEQ ID NO: 356 - RNA, 23 nt, Homo sapiens
  source 1..23: mol_type = transcribed RNA
tactgcatcaggaactgattgga

SEQ ID NO: 357 - RNA, 22 nt, Homo sapiens
  source 1..22: mol_type = transcribed RNA
tactcaggagagtggcaatcac

SEQ ID NO: 358 - RNA, 22 nt, Homo sapiens
  source 1..22: mol_type = transcribed RNA
aaaagtaatcactgtttttgcc

SEQ ID NO: 359 - RNA, 21 nt, Homo sapiens
  source 1..21: mol_type = transcribed RNA
gcttgtcgctgcggtgttgct

SEQ ID NO: 360 - RNA, 22 nt, Homo sapiens
  source 1..22: mol_type = transcribed RNA
tctgtgatagagattctttgct

SEQ ID NO: 361 - RNA, 23 nt, Homo sapiens
  source 1..23: mol_type = transcribed RNA
tgtgcaaatccatgcaaaactga

SEQ ID NO: 362 - RNA, 22 nt, Homo sapiens
  source 1..22: mol_type = transcribed RNA
atagtggttgtgaatttacctt

SEQ ID NO: 363 - RNA, 22 nt, Homo sapiens
  source 1..22: mol_type = transcribed RNA
tgatatgtttgatattgggttg

SEQ ID NO: 364 - RNA, 25 nt, Homo sapiens
  source 1..25: mol_type = transcribed RNA
aagggaggaggagcggaggggccct

SEQ ID NO: 365 - RNA, 22 nt, Homo sapiens
  source 1..22: mol_type = transcribed RNA
tggtctaggattgttggaggag

SEQ ID NO: 366 - RNA, 24 nt, Homo sapiens
  source 1..24: mol_type = transcribed RNA
agagctcacagctgtccttctcta

SEQ ID NO: 367 - RNA, 21 nt, Homo sapiens
  source 1..21: mol_type = transcribed RNA
ggttcttagcataggaggtct

SEQ ID NO: 368 - RNA, 24 nt, Homo sapiens
  source 1..24: mol_type = transcribed RNA
agcagaagcagggaggttctccca

SEQ ID NO: 369 - RNA, 23 nt, Homo sapiens
  source 1..23: mol_type = transcribed RNA
ggggaaagcgagtagggacattt

SEQ ID NO: 370 - RNA, 22 nt, Homo sapiens
  source 1..22: mol_type = transcribed RNA
agggaaggaggcttggtcttag

SEQ ID NO: 371 - RNA, 22 nt, Homo sapiens
  source 1..22: mol_type = transcribed RNA
tgattggtacgtctgtgggtag

SEQ ID NO: 372 - RNA, 17 nt, Homo sapiens
  source 1..17: mol_type = transcribed RNA
acattgccagggagttt

SEQ ID NO: 373 - RNA, 22 nt, Homo sapiens
  source 1..22: mol_type = transcribed RNA
agcaaaataagcaaatggaaaa

SEQ ID NO: 374 - RNA, 21 nt, Homo sapiens
  source 1..21: mol_type = transcribed RNA
cagtgatttgaggattattgc

SEQ ID NO: 375 - RNA, 21 nt, Homo sapiens
  source 1..21: mol_type = transcribed RNA
ttggacggtaaggttaagcaa

SEQ ID NO: 376 - RNA, 17 nt, Homo sapiens
  source 1..17: mol_type = transcribed RNA
ttctgagctgaggacag

SEQ ID NO: 377 - RNA, 21 nt, Homo sapiens
  source 1..21: mol_type = transcribed RNA
actggcattagtgggactttt

SEQ ID NO: 378 - RNA, 21 nt, Homo sapiens
  source 1..21: mol_type = transcribed RNA
ggcgcgcccagctcccgggct

SEQ ID NO: 379 - RNA, 22 nt, Homo sapiens
  source 1..22: mol_type = transcribed RNA
tatccagcttgttactatatgc

SEQ ID NO: 380 - RNA, 22 nt, Homo sapiens
  source 1..22: mol_type = transcribed RNA
tgttgtacttttttttttgttc

SEQ ID NO: 381 - RNA, 21 nt, Homo sapiens
  source 1..21: mol_type = transcribed RNA
tagtagaccgtatagcgtacg

SEQ ID NO: 382 - RNA, 17 nt, Homo sapiens
  source 1..17: mol_type = transcribed RNA
gtggacctggctgggac

SEQ ID NO: 383 - RNA, 20 nt, Homo sapiens
  source 1..20: mol_type = transcribed RNA
accaggaggctgaggcccct

SEQ ID NO: 384 - RNA, 21 nt, Homo sapiens
  source 1..21: mol_type = transcribed RNA
taacgcataatatggacatgt

SEQ ID NO: 385 - RNA, 23 nt, Homo sapiens
  source 1..23: mol_type = transcribed RNA
taaatttcacctttctgagaaga

SEQ ID NO: 386 - RNA, 21 nt, Homo sapiens
  source 1..21: mol_type = transcribed RNA
attgcctaacatgtgccagaa

SEQ ID NO: 387 - RNA, 21 nt, Homo sapiens
  source 1..21: mol_type = transcribed RNA
ccgtcgccgccacccgagccg

SEQ ID NO: 388 - RNA, 20 nt, Homo sapiens
  source 1..20: mol_type = transcribed RNA
atggagatagatatagaaat

SEQ ID NO: 389 - RNA, 23 nt, Homo sapiens
  source 1..23: mol_type = transcribed RNA
tggccggatgggacaggaggcat

SEQ ID NO: 390 - RNA, 23 nt, Homo sapiens
  source 1..23: mol_type = transcribed RNA
tggtgggtggggaggagaagtgc

SEQ ID NO: 391 - RNA, 19 nt, Homo sapiens
  source 1..19: mol_type = transcribed RNA
aggctgcggaattcaggac

SEQ ID NO: 392 - RNA, 22 nt, Homo sapiens
  source 1..22: mol_type = transcribed RNA
tgaaactggagcgcctggagga

SEQ ID NO: 393 - RNA, 23 nt, Homo sapiens
  source 1..23: mol_type = transcribed RNA
aggaagccctggaggggctggag

SEQ ID NO: 394 - RNA, 21 nt, Homo sapiens
  source 1..21: mol_type = transcribed RNA
tcatccccctcgccctctcag

SEQ ID NO: 395 - RNA, 22 nt, Homo sapiens
  source 1..22: mol_type = transcribed RNA
gctctgactttattgcactact

SEQ ID NO: 396 - RNA, 23 nt, Homo sapiens
  source 1..23: mol_type = transcribed RNA
caaagtgcttacagtgcaggtag

SEQ ID NO: 397 - RNA, 22 nt, Homo sapiens
  source 1..22: mol_type = transcribed RNA
tggtgggcacagaatctggact

SEQ ID NO: 398 - RNA, 22 nt, Homo sapiens
  source 1..22: mol_type = transcribed RNA
ttctagatgagagatatatata

SEQ ID NO: 399 - RNA, 20 nt, Homo sapiens
  source 1..20: mol_type = transcribed RNA
tcactacctgacaatacagt

SEQ ID NO: 400 - RNA, 22 nt, Homo sapiens
  source 1..22: mol_type = transcribed RNA
attaaggacatttgtgattgat

SEQ ID NO: 401 - RNA, 21 nt, Homo sapiens
  source 1..21: mol_type = transcribed RNA
tgctgtattgtcaggtagtga

SEQ ID NO: 402 - RNA, 19 nt, Homo sapiens
  source 1..19: mol_type = transcribed RNA
ttgatctcggaagctaagc

SEQ ID NO: 403 - RNA, 22 nt, Homo sapiens
  source 1..22: mol_type = transcribed RNA
caaataataccacagtgggtgt

SEQ ID NO: 404 - RNA, 21 nt, Homo sapiens
  source 1..21: mol_type = transcribed RNA
tgggggtgtggggagagagag

SEQ ID NO: 405 - RNA, 22 nt, Homo sapiens
  source 1..22: mol_type = transcribed RNA
tggaaggaggttgccggacgct

SEQ ID NO: 406 - RNA, 22 nt, Homo sapiens
  source 1..22: mol_type = transcribed RNA
tgcgacattggaagtagtatca

SEQ ID NO: 407 - RNA, 22 nt, Homo sapiens
  source 1..22: mol_type = transcribed RNA
ctgggagaaggctgtttactct

SEQ ID NO: 408 - RNA, 22 nt, Homo sapiens
  source 1..22: mol_type = transcribed RNA
ttggacagaaaacacgcaggaa

SEQ ID NO: 409 - RNA, 22 nt, Homo sapiens
  source 1..22: mol_type = transcribed RNA
ctagtgctctccgttacaagta

SEQ ID NO: 410 - RNA, 20 nt, Homo sapiens
  source 1..20: mol_type = transcribed RNA
ccaggagatccagagagaat

SEQ ID NO: 411 - RNA, 20 nt, Homo sapiens
  source 1..20: mol_type = transcribed RNA
gaggcagaagcaggatgaca

SEQ ID NO: 412 - RNA, 23 nt, Homo sapiens
  source 1..23: mol_type = transcribed RNA
atggagctggaaccagatcaggc

SEQ ID NO: 413 - RNA, 21 nt, Homo sapiens
  source 1..21: mol_type = transcribed RNA
tcgtgcatatatctaccacat

SEQ ID NO: 414 - RNA, 22 nt, Homo sapiens
  source 1..22: mol_type = transcribed RNA
aagttggctgcagttaaggtgg

SEQ ID NO: 415 - RNA, 20 nt, Homo sapiens
  source 1..20: mol_type = transcribed RNA
taaagagccctgtggagaca

SEQ ID NO: 416 - RNA, 21 nt, Homo sapiens
  source 1..21: mol_type = transcribed RNA
gtgagggactgggatttgtgg

SEQ ID NO: 417 - RNA, 20 nt, Homo sapiens
  source 1..20: mol_type = transcribed RNA
aatgtgtagcaaaagacaga

SEQ ID NO: 418 - RNA, 21 nt, Homo sapiens
  source 1..21: mol_type = transcribed RNA
tgtgtcccattattggtgatt

SEQ ID NO: 419 - RNA, 21 nt, Homo sapiens
  source 1..21: mol_type = transcribed RNA
caaaacgtgaggcgctgctat

SEQ ID NO: 420 - RNA, 18 nt, Homo sapiens
  source 1..18: mol_type = transcribed RNA
gctggtgacatgagaggc

SEQ ID NO: 421 - RNA, 22 nt, Homo sapiens
  source 1..22: mol_type = transcribed RNA
gactgacacctctttgggtgaa

SEQ ID NO: 422 - RNA, 22 nt, Homo sapiens
  source 1..22: mol_type = transcribed RNA
ctgtgcgtgtgacagcggctga

SEQ ID NO: 423 - RNA, 23 nt, Homo sapiens
  source 1..23: mol_type = transcribed RNA
gtgagtgggagccggtggggctg

SEQ ID NO: 424 - RNA, 22 nt, Homo sapiens
  source 1..22: mol_type = transcribed RNA
gtagcaccttgcaggataaggt

SEQ ID NO: 425 - RNA, 17 nt, Homo sapiens
  source 1..17: mol_type = transcribed RNA
ggggctgggcgcgcgcc

SEQ ID NO: 426 - RNA, 26 nt, Homo sapiens
  source 1..26: mol_type = transcribed RNA
gtgagggcatgcaggcctggatgggg

SEQ ID NO: 427 - RNA, 23 nt, Homo sapiens
  source 1..23: mol_type = transcribed RNA
tttgggattgacgccacatgtct

SEQ ID NO: 428 - RNA, 22 nt, Homo sapiens
  source 1..22: mol_type = transcribed RNA
tacaagtcaggagctgaagcag

SEQ ID NO: 429 - RNA, 23 nt, Homo sapiens
  source 1..23: mol_type = transcribed RNA
atgagactcatgtaaaacatctt

SEQ ID NO: 430 - RNA, 22 nt, Homo sapiens
  source 1..22: mol_type = transcribed RNA
ctgccctggcccgagggaccga

SEQ ID NO: 431 - RNA, 22 nt, Homo sapiens
  source 1..22: mol_type = transcribed RNA
tctgggcaacaaagtgagacct

SEQ ID NO: 432 - RNA, 21 nt, Homo sapiens
  source 1..21: mol_type = transcribed RNA
gtttcaccatgttggtcaggc

SEQ ID NO: 433 - RNA, 22 nt, Homo sapiens
  source 1..22: mol_type = transcribed RNA
tatgtagtagtcaaaggcattt

SEQ ID NO: 434 - RNA, 22 nt, Homo sapiens
  source 1..22: mol_type = transcribed RNA
agacttcccatttgaaggtggc

SEQ ID NO: 435 - RNA, 20 nt, Homo sapiens
  source 1..20: mol_type = transcribed RNA
agagtcttgtgatgtcttgc

SEQ ID NO: 436 - RNA, 24 nt, Homo sapiens
  source 1..24: mol_type = transcribed RNA
tgtaggcatgaggcagggcccagg

SEQ ID NO: 437 - RNA, 21 nt, Homo sapiens
  source 1..21: mol_type = transcribed RNA
aaggcagggcccccgctcccc

SEQ ID NO: 438 - RNA, 20 nt, Homo sapiens
  source 1..20: mol_type = transcribed RNA
tcccactacttcacttgtga

SEQ ID NO: 439 - RNA, 16 nt, Homo sapiens
  source 1..16: mol_type = transcribed RNA
tccagctcggtggcac

SEQ ID NO: 440 - RNA, 22 nt, Homo sapiens
  source 1..22: mol_type = transcribed RNA
aggtgttatcctatccatttgc

SEQ ID NO: 441 - RNA, 17 nt, Homo sapiens
  source 1..17: mol_type = transcribed RNA
ccggcatgtccagggca

SEQ ID NO: 442 - RNA, 18 nt, Homo sapiens
  source 1..18: mol_type = transcribed RNA
cgtgtcttctggcttgat

SEQ ID NO: 443 - RNA, 23 nt, Homo sapiens
  source 1..23: mol_type = transcribed RNA
tgaggctctgttagccttggctc

SEQ ID NO: 444 - RNA, 22 nt, Homo sapiens
  source 1..22: mol_type = transcribed RNA
agaggtagtaggttgcatagtt

SEQ ID NO: 445 - RNA, 24 nt, Homo sapiens
  source 1..24: mol_type = transcribed RNA
tatatggacttttctgatacaatg

SEQ ID NO: 446 - RNA, 23 nt, Homo sapiens
  source 1..23: mol_type = transcribed RNA
tgtttcggggctcatggcctgtg

SEQ ID NO: 447 - RNA, 23 nt, Homo sapiens
  source 1..23: mol_type = transcribed RNA
tgcagctctggtggaaaatggag

SEQ ID NO: 448 - RNA, 22 nt, Homo sapiens
  source 1..22: mol_type = transcribed RNA
tcagatgatctaaaggcctata

SEQ ID NO: 449 - RNA, 22 nt, Homo sapiens
  source 1..22: mol_type = transcribed RNA
cctggcatatttggtataactt

SEQ ID NO: 450 - RNA, 23 nt, Homo sapiens
  source 1..23: mol_type = transcribed RNA
taccctgtagaaccgaatttgtg

SEQ ID NO: 451 - RNA, 20 nt, Homo sapiens
  source 1..20: mol_type = transcribed RNA
tcgcgccccggctcccgttc

SEQ ID NO: 452 - RNA, 22 nt, Homo sapiens
  source 1..22: mol_type = transcribed RNA
tgaggtaggaggttgtatagtt

SEQ ID NO: 453 - RNA, 22 nt, Homo sapiens
  source 1..22: mol_type = transcribed RNA
ttaattttttgtttcggtcact

SEQ ID NO: 454 - RNA, 21 nt, Homo sapiens
  source 1..21: mol_type = transcribed RNA
tctgaaagagcagttggtgtt

SEQ ID NO: 455 - RNA, 22 nt, Homo sapiens
  source 1..22: mol_type = transcribed RNA
gcctgctggggtggaacctggt

SEQ ID NO: 456 - RNA, 21 nt, Homo sapiens
  source 1..21: mol_type = transcribed RNA
acagtctgctgaggttggagc

SEQ ID NO: 457 - RNA, 21 nt, Homo sapiens
  source 1..21: mol_type = transcribed RNA
cacacatagcaggtgtatata

SEQ ID NO: 458 - RNA, 22 nt, Homo sapiens
  source 1..22: mol_type = transcribed RNA
aacccgtagatccgaacttgtg

SEQ ID NO: 459 - RNA, 22 nt, Homo sapiens
  source 1..22: mol_type = transcribed RNA
taaatagagtaggcaaaggaca

SEQ ID NO: 460 - RNA, 22 nt, Homo sapiens
  source 1..22: mol_type = transcribed RNA
tttcttcttagacatggcagct

SEQ ID NO: 461 - RNA, 22 nt, Homo sapiens
  source 1..22: mol_type = transcribed RNA
tgagacctctgggttctgagct

SEQ ID NO: 462 - RNA, 22 nt, Homo sapiens
  source 1..22: mol_type = transcribed RNA
agagttgagtctggacgtcccg

SEQ ID NO: 463 - RNA, 23 nt, Homo sapiens
  source 1..23: mol_type = transcribed RNA
tgtgactgcattatgaaaattct

SEQ ID NO: 464 - RNA, 22 nt, Homo sapiens
  source 1..22: mol_type = transcribed RNA
ctgggggtggggggctgggcgt

SEQ ID NO: 465 - RNA, 22 nt, Homo sapiens
  source 1..22: mol_type = transcribed RNA
cacacaagtggcccccaacact

SEQ ID NO: 466 - RNA, 22 nt, Homo sapiens
  source 1..22: mol_type = transcribed RNA
gatgagctcattgtaatatgag

SEQ ID NO: 467 - RNA, 22 nt, Homo sapiens
  source 1..22: mol_type = transcribed RNA
aaggggcagggacgggtggccc

SEQ ID NO: 468 - RNA, 22 nt, Homo sapiens
  source 1..22: mol_type = transcribed RNA
tgcaacttacctgagtcattga

SEQ ID NO: 469 - RNA, 22 nt, Homo sapiens
  source 1..22: mol_type = transcribed RNA
atgctgacatatttactagagg

SEQ ID NO: 470 - RNA, 21 nt, Homo sapiens
  source 1..21: mol_type = transcribed RNA
gtaagcaggggctctgggtga

SEQ ID NO: 471 - RNA, 22 nt, Homo sapiens
  source 1..22: mol_type = transcribed RNA
tagcaccatttgaaatcggtta

SEQ ID NO: 472 - RNA, 23 nt, Homo sapiens
  source 1..23: mol_type = transcribed RNA
ggcaggagggctgtgccaggttg

SEQ ID NO: 473 - RNA, 22 nt, Homo sapiens
  source 1..22: mol_type = transcribed RNA
ttttcaactctaatgggagaga

SEQ ID NO: 474 - RNA, 22 nt, Homo sapiens
  source 1..22: mol_type = transcribed RNA
gcagtagtgtagagattggttt

SEQ ID NO: 475 - RNA, 21 nt, Homo sapiens
  source 1..21: mol_type = transcribed RNA
tgagtgttgtctacgagggca

SEQ ID NO: 476 - RNA, 22 nt, Homo sapiens
  source 1..22: mol_type = transcribed RNA
atatacctgttcggtctcttta

SEQ ID NO: 477 - RNA, 22 nt, Homo sapiens
  source 1..22: mol_type = transcribed RNA
taggggcagcagaggacctggg

SEQ ID NO: 478 - RNA, 22 nt, Homo sapiens
  source 1..22: mol_type = transcribed RNA
tggggaggtgtggagtcagcat

SEQ ID NO: 479 - RNA, 22 nt, Homo sapiens
  source 1..22: mol_type = transcribed RNA
aaaggaaagtgtatcctaaaag

SEQ ID NO: 480 - RNA, 22 nt, Homo sapiens
  source 1..22: mol_type = transcribed RNA
agcccgccccagccgaggttct

SEQ ID NO: 481 - RNA, 22 nt, Homo sapiens
  source 1..22: mol_type = transcribed RNA
aactggatcaattataggagtg

SEQ ID NO: 482 - RNA, 19 nt, Homo sapiens
  source 1..19: mol_type = transcribed RNA
tggatgacagtggaggcct

SEQ ID NO: 483 - RNA, 22 nt, Homo sapiens
  source 1..22: mol_type = transcribed RNA
cagcaccctgtggctcccacag

SEQ ID NO: 484 - RNA, 22 nt, Homo sapiens
  source 1..22: mol_type = transcribed RNA
gtcactgatgtctgtagctgag

SEQ ID NO: 485 - RNA, 22 nt, Homo sapiens
  source 1..22: mol_type = transcribed RNA
ttgtgtcaatatgcgatgatgt

SEQ ID NO: 486 - RNA, 23 nt, Homo sapiens
  source 1..23: mol_type = transcribed RNA
tctggcaagtaaaaaactctcat

SEQ ID NO: 487 - RNA, 22 nt, Homo sapiens
  source 1..22: mol_type = transcribed RNA
tagtcccttccttgaagcggtc

SEQ ID NO: 488 - RNA, 23 nt, Homo sapiens
  source 1..23: mol_type = transcribed RNA
tctgtattctcctttgcctgcag

SEQ ID NO: 489 - RNA, 22 nt, Homo sapiens
  source 1..22: mol_type = transcribed RNA
atcaaataaggactagtctgca

SEQ ID NO: 490 - RNA, 22 nt, Homo sapiens
  source 1..22: mol_type = transcribed RNA
ttggtgaggaccccaagctcgg

SEQ ID NO: 491 - RNA, 21 nt, Homo sapiens
  source 1..21: mol_type = transcribed RNA
ttgccagggcaggaggtggaa

SEQ ID NO: 492 - RNA, 23 nt, Homo sapiens
  source 1..23: mol_type = transcribed RNA
agggaaaaaaaaaaggatttgtc

SEQ ID NO: 493 - RNA, 22 nt, Homo sapiens
  source 1..22: mol_type = transcribed RNA
aagagaactgaaagtggagcct

SEQ ID NO: 494 - RNA, 22 nt, Homo sapiens
  source 1..22: mol_type = transcribed RNA
tttaagaaaacaccatggagat

SEQ ID NO: 495 - RNA, 23 nt, Homo sapiens
  source 1..23: mol_type = transcribed RNA
agggtggggctggaggtggggct

SEQ ID NO: 496 - RNA, 20 nt, Homo sapiens
  source 1..20: mol_type = transcribed RNA
gtgcaaaagtcatcacggtt

SEQ ID NO: 497 - RNA, 22 nt, Homo sapiens
  source 1..22: mol_type = transcribed RNA
aacacacctattcaaggattca

SEQ ID NO: 498 - RNA, 22 nt, Homo sapiens
  source 1..22: mol_type = transcribed RNA
tagggggcggcttgtggagtgt

SEQ ID NO: 499 - RNA, 22 nt, Homo sapiens
  source 1..22: mol_type = transcribed RNA
cttctgatcaagatttgtggtg

SEQ ID NO: 500 - RNA, 22 nt, Homo sapiens
  source 1..22: mol_type = transcribed RNA
cagagggaatacagagggcaat